(19) United States
(12) Patent Application Publication
(10) Pub. No.: US 2025/0011399 A1
(43) Pub. Date: Jan. 9, 2025

(54) CHIMERIC ANTIGEN RECEPTORS

(71) Applicant: Outpace Bio, Inc., Seattle, WA (US)

(72) Inventors: Yun SONG, Seattle, WA (US); Marc Joseph LAJOIE, Seattle, WA (US); Brian Douglas WEITZNER, Colorado Springs, CO (US); Scott Edward BOYKEN, Seattle, WA (US)

(21) Appl. No.: 18/708,558

(22) PCT Filed: Nov. 10, 2022
(86) PCT No.: PCT/US2022/049611

§ 371 Date: May 8, 2024

Related U.S. Application Data
(60) Provisional application No. 63/307,612, filed on Feb. 7, 2022.
(60) Provisional application No. 63/277,984, filed on Nov. 10, 2021.

(51) Int. Cl. C07K 16/10; A61K 39/00; C07K 14/705; C07K 14/725

(52) U.S. Cl. CPC C07K 16/1018; A61K 39/4611; A61K 39/4631; A61K 39/464838; C07K 14/7051; C07K 14/70521; C07K 2317/53; C07K 2317/622; C07K 2317/73; C07K 2319/02; C07K 2319/03

(57) ABSTRACT

Provided herein are chimeric antigen receptors (CAR) that include a spacer sequence that provides for a cell-to-cell distance between a cell expressing the CAR and a target cell. Embodiments are provided in which an extracellular element of CARs include a spacer sequence, wherein, when the CAR is expressed on the surface of a cell, a cell-to-cell distance between the CAR expressing cell and a target expressing cell is between about 17 nm to about 25 nm, inclusive of the endpoints.

### FIELD OF THE DISCLOSURE

The disclosure relates to chimeric antigen receptors (CARs) comprising spacer domains, compositions comprising CARs, and sequences encoding CARs as well as methods of use.

### CROSS REFERENCE

This application claims the benefit of U.S. Provisional Patent Application No. 63/277,984, filed on Nov. 10, 2021 and U.S. Provisional Patent Application No. 63/307,612, filed on Feb. 7, 2022, which is each incorporated by reference in its entirety.

### BACKGROUND

Chimeric antigen receptors (CARs) are synthetic receptors that can be used to provide immune cells with engineered specificity. The use of CARs to redirect T cells to recognize a target cell is a promising approach to cancer immunotherapy. CAR design has focused on identifying antigen binding domains and defining intracellular signaling modules to activate T-cell effector functions. The disclosure provides CARs, compositions and methods for use of CARs incorporating a spacer to achieve superior CAR activity and, therefore, superior efficacy for cellular therapies in which cells express these CARs on their surface.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### SUMMARY

The disclosure provides a Chimeric Antigen Receptor (CAR) comprising (a) an extracellular element comprising (1) a binding element that specifically binds to a target and (2) a spacer sequence; (b) a transmembrane element; and (c) an intracellular element; wherein, when the CAR is expressed on the surface of a cell, a cell-to-cell distance between a CAR expressing cell and a target expressing cell is between about 17 nm to about 25 nm, inclusive of the endpoints. In some embodiments, when the CAR is expressed on the surface of a cell, a cell-to-cell distance between a CAR expressing cell and a target expressing cell is between from about 17 nm to about 22 nm. In some embodiments, when the CAR is expressed on the surface of a cell, a cell-to-cell distance between a CAR expressing cell and a target expressing cell is between from about 17 nm to about 23 nm. In some embodiments, when the CAR is expressed on the surface of a cell, a cell-to-cell distance between a CAR expressing cell and a target expressing cell is between from about 18 nm to about 22 nm. In some embodiments, when the CAR is expressed on the surface of a cell, a cell-to-cell distance between a CAR expressing cell and a target expressing cell is about 20 nm. In some embodiments, the target comprises an epitope. In some embodiments, the target comprises a linear epitope, a continuous epitope, a discontinuous epitope, and/or a conformational epitope. In some embodiments, the target comprises an antigen. In some embodiments, the target comprises an amino acid sequence. In some embodiments, the target does not comprise an amino acid.

In some embodiments of the CARs of the disclosure, including those comprising (a) an extracellular element comprising (1) a binding element that specifically binds to a target and (2) a spacer sequence; (b) a transmembrane element; and (c) an intracellular element; wherein, when the CAR is expressed on the surface of a cell, a cell-to-cell distance between a CAR expressing cell and a target expressing cell is between about 17 nm to about 25 nm, inclusive of the endpoints, and the spacer sequence comprises a length of between about 2 nanometers (nm) and about 20 nm, inclusive of the endpoints. In some embodiments, the spacer sequence comprises a length of between about 3 nm and 20 nm, inclusive of the endpoints In some embodiments, the spacer sequence comprises a length of between about 4 nm and 20 nm, inclusive of the endpoints.

In some embodiments of the CARs of the disclosure, including those comprising (a) an extracellular element comprising (1) a binding element that specifically binds to a target and (2) a spacer sequence; (b) a transmembrane element; and (c) an intracellular element; wherein, when the CAR is expressed on the surface of a cell, a cell-to-cell distance between a CAR expressing cell and a target expressing cell is, in each case, inclusive of the endpoints: between about 17 nm and about 18 nm; between about 18 nm and about 19 nm; between about 19 nm and about 20 nm; between about 20 nm and about 21 nm; between about 21 nm and about 22 nm; between about 22 nm and about 23 nm; between about 23 nm and about 24 nm; or between about 24 nm and about 25 nm.

In some embodiments of the CARs of the disclosure, including those comprising (a) an extracellular element comprising (1) a binding element that specifically binds to a target and (2) a spacer sequence; (b) a transmembrane element; and (c) an intracellular element; wherein, when the CAR is expressed on the surface of a cell, a cell-to-cell distance between a CAR expressing cell and a target expressing cell is, in each case, inclusive of the endpoints: between about 17 nm and about 25 nm; between about 18 nm and about 24 nm; between about 19 nm and about 23 nm; between about 20 nm and about 22 nm; or between about 21 nm and about 23 nm.

In some embodiments of the CARs of the disclosure, including those comprising (a) an extracellular element comprising (1) a binding element that specifically binds to a target and (2) a spacer sequence; (b) a transmembrane element; and (c) an intracellular element; wherein, when the CAR is expressed on the surface of a cell, a cell-to-cell distance between a CAR expressing cell and a target expressing cell is, in each case: about 17 nm, about 18 nm, about 19 nm, about 20 nm, about 21 nm, about 22 nm, about 23 nm, about 24 nm, or about 25 nm.

In some embodiments of the CARs of the disclosure, including those comprising (a) an extracellular element comprising (1) a binding element that specifically binds to a target and (2) a spacer sequence; (b) a transmembrane element; and (c) an intracellular element; wherein, when the CAR is expressed on the surface of a cell, a cell-to-cell distance between a CAR expressing cell and a target expressing cell is, in each case: 17 nm, 18 nm, 19 nm, 20 nm, 21 nm, 22 nm, 23 nm, 24 nm, or 25 nm.

In some embodiments of the CARs of the disclosure, including those comprising (a) an extracellular element comprising (1) a binding element that specifically binds to a target and (2) a spacer sequence; (b) a transmembrane element; and (c) an intracellular element; wherein, when the CAR is expressed on the surface of a cell, a cell-to-cell distance between a CAR expressing cell and a target expressing cell is, in each case, inclusive of the endpoints: between about 17 nm and about 19 nm; between about 18 nm and about 20 nm; between about 19 nm and about 21 nm; between about 20 nm and about 22 nm; between about 21 nm and about 23 nm; between about 22 nm and about 24 nm; or between about 23 nm and about 25 nm.

In some embodiments of the CARs of the disclosure, including those comprising (a) an extracellular element comprising (1) a binding element that specifically binds to a target and (2) a spacer sequence; (b) a transmembrane element; and (c) an intracellular element; wherein, when the CAR is expressed on the surface of a cell, a cell-to-cell distance between a CAR expressing cell and a target expressing cell is between about 17 nm to about 25 nm, inclusive of the endpoints, the spacer sequence comprises a linker sequence. In some embodiments, the linker comprises a Glycine-Serine (GS) linker. In some embodiments, the linker sequence comprises GGGSG (SEQ ID NO: 77).

In some embodiments of the CARs of the disclosure, including those comprising (a) an extracellular element comprising (1) a binding element that specifically binds to a target and (2) a spacer sequence; (b) a transmembrane element; and (c) an intracellular element; wherein, when the CAR is expressed on the surface of a cell, a cell-to-cell distance between a CAR expressing cell and a target expressing cell is between about 17 nm to about 25 nm, inclusive of the endpoints, the extracellular element comprises a linker sequence. In some embodiments, the linker comprises a Glycine-Serine (GS) linker. In some embodiments, the linker sequence comprises GGGSG (SEQ ID NO: 77).

In some embodiments of the CARs of the disclosure, including those comprising (a) an extracellular element comprising (1) a binding element that specifically binds to a target and (2) a spacer sequence; (b) a transmembrane element; and (c) an intracellular element; wherein, when the CAR is expressed on the surface of a cell, a cell-to-cell distance between a CAR expressing cell and a target expressing cell is between about 17 nm to about 25 nm, inclusive of the endpoints, the intracellular element comprises a linker sequence. In some embodiments, the linker comprises a Glycine-Serine (GS) linker. In some embodiments, the linker sequence comprises GGGSG (SEQ ID NO: 77).

In some embodiments of the CARs of the disclosure, including those comprising (a) an extracellular element comprising (1) a binding element that specifically binds to a target and (2) a spacer sequence; (b) a transmembrane element; and (c) an intracellular element; wherein, when the CAR is expressed on the surface of a cell, a cell-to-cell distance between a CAR expressing cell and a target expressing cell is between about 17 nm to about 25 nm, inclusive of the endpoints, the spacer sequence comprises one or more sequences selected from the group consisting of SEQ ID NO: 1-44. In some embodiments, the spacer sequence comprises any one of sequences SEQ ID NO: 1-44.

The disclosure provides a nucleic acid sequence encoding the CAR of the disclosure, or any element thereof. In some embodiments, the nucleic acid sequence further comprises one or more regulatory sequences. In some embodiments, the regulatory sequence comprises a human sequence. In some embodiments, the regulatory sequence comprises a sequence isolated or derived from a human sequence. In some embodiments, the regulatory sequence comprises a sequence isolated or derived from a mammalian sequence. In some embodiments, the nucleic acid sequence further comprises at least one repeat of a regulatory sequence. In some embodiments, the nucleic acid sequence further comprises at least two repeats of a regulatory sequence and a linking sequence positioned between the two repeats. In some embodiments, the one or more regulatory sequences include, but are not limited to, sequences encoding, comprising or consisting of a response element, a promoter, an enhancer, a repressor, an insulator, a silencer, an intron, an exon, an untranslated region (UTR), a 5′ UTR, a 3′ UTR, a post-translational regulatory sequence (PRE), or any minimal functional sequence thereof. In some embodiments, the response element comprises an inducible response element. In some embodiments, the promoter comprises an inducible promoter. Alternatively, or in addition, the promoter comprises a minimal promoter. In some embodiments, the PRE comprises a woodchuck PRE (WPRE).

The disclosure provides a vector comprising a nucleic acid sequence of the disclosure, including nucleic acid sequences encoding a CAR of the disclosure. In some embodiments, the vector further comprises one or more regulatory sequences of the disclosure. In some embodiments, the regulatory sequence comprises a human sequence. In some embodiments, the regulatory sequence comprises a sequence isolated or derived from a human sequence. In some embodiments, the regulatory sequence comprises a sequence isolated or derived from a mammalian sequence. In some embodiments, the nucleic acid sequence further comprises at least one repeat of a regulatory sequence. In some embodiments, the nucleic acid sequence further comprises at least two repeats of a regulatory sequence and a linking sequence positioned between the two repeats. In some embodiments, the one or more regulatory sequences include, but are not limited to, sequences encoding, comprising or consisting of a response element, a promoter, an enhancer, a repressor, an insulator, a silencer, an intron, an exon, an untranslated region (UTR), a 5′ UTR, a 3′ UTR, a post-translational regulatory sequence (PRE), or any minimal functional sequence thereof. In some embodiments, the response element comprises an inducible response element. In some embodiments, the promoter comprises an inducible promoter. Alternatively, or in addition, the promoter comprises a minimal promoter. In some embodiments, the PRE comprises a woodchuck PRE (WPRE). In some embodiments, the vector further comprises one or more integration sequences, including but not limited to, terminal repeat sequence(s), inverted terminal repeat sequence(s), long terminal repeat sequence(s), sequence(s) homologous to a target sequence, sequence(s)s capable of facilitating homologous recombination, transposable element(s), and/or transposon sequence(s). In some embodiments, the vector further comprises one or more inverted terminal repeat sequences (ITRs). In some embodiments, the vector further comprises a sequence encoding a selection element. In some embodiments, the vector further comprises a polycistronic or a self-cleaving sequence. In some embodiments, including those in which the vector further comprises a polycistronic or a self-cleaving sequence the vector further comprises a sequence encoding a selection element. In some embodiments, including those in which the vector further comprises a polycistronic or a self-cleaving sequence the vector further comprises a safety switch. In some embodiments, including those in which the vector further comprises a polycistronic or a self-cleaving sequence the vector further comprises a second therapeutically effective agent. In some embodiments, the self-cleaving sequence comprises a 2A self-cleaving sequence. In some embodiments, the 2A self-cleaving sequence comprises a foot-and-mouth disease virus (F2A) sequence, an equine rhinitis A virus (E2A) sequence, a porcine teschovirus-1 2A (P2A) sequence, or a thosea asigna virus 2 (T2A) sequence. In some embodiments, the 2A self-cleaving sequence comprises GDVEXNPGP (SEQ ID NO: 80). In some embodiments, the P2A self-cleaving sequence comprises ATNFSLLKQAGDVEENPGP (SEQ ID NO: 81). In some embodiments, the T2A self-cleaving sequence comprises EGRGSLLTLGDVEENPGP (SEQ ID NO: 83). In some embodiments, the E2A self-cleaving sequence comprises QCTNYALLKLAGDVESNPGP (SEQ ID NO: 84).

The disclosure provides a composition comprising a CAR of the disclosure, or any element thereof. In some embodiments, a CAR of the disclosure comprises a “first generation” CAR. In some embodiments, a CAR of the disclosure comprises an extracellular element, a transmembrane element, and an intracellular element, wherein the intracellular element comprises a CD3 signaling element. In some embodiments, a CAR of the disclosure comprises a “second generation” CAR. In some embodiments, a CAR of the disclosure comprises an extracellular element, a transmembrane element, and an intracellular element, wherein the intracellular element comprises a CD28 signaling element and a CD3 signaling element. In some embodiments, a CAR of the disclosure comprises a “third generation” CAR. In some embodiments, a CAR of the disclosure comprises an extracellular element, a transmembrane element, and an intracellular element, wherein the intracellular element comprises a CD28 signaling element, a 41BB signaling element and a CD3 signaling element. In some embodiments, a CAR of the disclosure comprises a “fourth generation” CAR. In some embodiments, a CAR of the disclosure comprises an extracellular element, a transmembrane element, and an intracellular element, wherein the intracellular element comprises a CD28 signaling element, a 41BB signaling element, an inducible element and a CD3 signaling element. In some embodiments, the inducible element responds to CAR activation by inducing expression of a transgenic sequence. In some embodiments of a CAR of the disclosure, the extracellular domain comprises a binding element wherein the binding element comprises a scFv. In all embodiments of a CAR of the disclosure, the extracellular element comprises a spacer sequence.

The disclosure provides a CARthat when expressed in a T-cell, transforms the modified cell into a TRUCK, a T-cell Redirected for Universal Cytokine-mediated Killing. In some embodiments, the CAR comprises a “fourth generation” CAR. In some embodiments, a CAR of the disclosure comprises an extracellular element, a transmembrane element, and an intracellular element, wherein the intracellular element comprises a CD28 signaling element, a 41BB signaling element, an inducible element and a CD3 signaling element. In some embodiments, the inducible element responds to CAR activation by inducing expression of a transgenic sequence. In some embodiments the inducible sequence encodes a cytokine.

The disclosure provides a composition comprising the nucleic acid of the disclosure.

The disclosure provides a composition comprising the vector of the disclosure. In some embodiments, the vector is an expression vector capable of expressing a nucleic acid sequence of the disclosure in a cell of the disclosure. In some embodiments, the expression vector is capable of expressing a nucleic acid sequence of the disclosure in a mammalian cell. In some embodiments, the expression vector is capable of expressing a nucleic acid sequence of the disclosure in a human cell. In some embodiments, the expression vector comprises a plasmid. In some embodiments, the vector is a delivery vector capable of introducing a nucleic acid sequence, an amino acid sequence, or a composition of the disclosure into a cell of the disclosure or into a subject of the disclosure. In some embodiments, the delivery vector comprises a viral vector. In some embodiments, the delivery vector comprises a non-viral vector.

The disclosure provides a cell comprising the CAR of the disclosure. In some embodiments, the cell is an immune cell or a precursor thereof. In some embodiments, the immune cell or the precursor is a mammalian cell. In some embodiments, the immune cell or the precursor is a human cell. In some embodiments, the immune cell or the precursor is a stem cell, an adult stem cell, a bone marrow cell, a hematopoietic stem cell (HSC), an induced pluripotent stem cell (iPSC), a de-differentiated cell or a transdifferentiated cell. In some embodiments, the stem cell is not a human embryonic stem cell. In some embodiments, the cell is a primary cell, a cultured cell, an immortalized cell or a cell isolated or derived from a cell line. In some embodiments, the immune cell is a T-cell, a B-cell, a natural killer (NK) cell, or a macrophage. In some embodiments, the T-cell is a memory T cell, an effector T cell, a helper T cell, or a natural killer (NK) T cell. In some embodiments, the T-cell is an alpha beta (αβ) T cell or a gamma delta (γδ) T cell. In some embodiments, the T-cell is an unstimulated T cell or a naïve T cell. In some embodiments, the T-cell is a stimulated T cell, a stem cell memory T cell (TSCM), a memory cell with naïve phenotypes (TMNP), or an effector T cell. In some embodiments, the memory T-cell is a stem cell memory T cell, a memory cell with naïve phenotypes (TMNP), a central memory T cell (TCM), an effector memory T cell (TEM), or a residual memory T cell (TRM). In some embodiments, the memory T-cell is a stimulated cell, having contacted one or more antigens or having one or more contacts with the same antigen, and the memory T cell is a stem cell memory T cell, a memory cell with naïve phenotypes (TMNP), a central memory T cell (TCM), an effector memory T cell (TEM), or a residual memory T cell (TRM).

The disclosure provides a cell comprising the nucleic acid ofthe disclosure.

The disclosure provides a cell comprising the vector of the disclosure.

The disclosure provides a composition comprising a cell of the disclosure.

The disclosure provides a pharmaceutical composition comprising a CAR of the disclosure and a pharmaceutically acceptable carrier.

The disclosure provides a pharmaceutical composition comprising a nucleic acid of claim 23 and a pharmaceutically acceptable carrier.

The disclosure provides a pharmaceutical composition comprising a vector of the disclosure. and a pharmaceutically acceptable carrier.

The disclosure provides a pharmaceutical composition comprising a cell of the disclosure and a pharmaceutically acceptable carrier.

In some embodiments of the disclosure, the pharmaceutical composition is formulated for systemic administration to a subject of the disclosure. In some embodiments, the pharmaceutical composition is formulated for systemic administration to a subject by intravenous injection or infusion. In some embodiments, the pharmaceutical composition is formulated for local administration to a subject of the disclosure. In some embodiments, the pharmaceutical composition is formulated for local administration to a subject by intratumoral injection or infusion. In some embodiments, the pharmaceutically acceptable carrier is a sterile solution comprising one or more agents to individually, or in combination, maintain or improve viability of a cellular therapy, reduce immunogenicity, reduce injection site discomfort of the subject, and/or maintain one or more of a physiological pH, cell density, and viscosity.

The disclosure provides a use of a CAR of the disclosure, a nucleic acid of the disclosure, a vector ofthe disclosure, a cell of any one ofthe disclosure, or a pharmaceutical composition of the disclosure for manufacture of a medicament. In some embodiments, the medicament is capable of or the medicament is suitable for treating a disease or disorder of the disclosure. In some embodiments, the medicament is formulated for use in treating a disease or disorder of the disclosure. In some embodiments, the CAR selectively targets an antigen or an epitope associated with the disease or disorder as a causal agent of the disease or disorder, a regulator of a causal agent of the disease or disorder, a biomarker of the disease or disorder, a sign of a level of severity of the disease or disorder, and/or a sign of a prognosis of the disease or disorder.

The disclosure provides a use of a CAR of the disclosure, a nucleic acid of the disclosure, a vector of the disclosure, a cell of any one of the disclosure, or a pharmaceutical composition of the disclosure for treatment or prevention of a disease or disorder. In some embodiments, the disease or disorder comprises a cancer. In some embodiments, the CAR selectively targets an antigen or an epitope associated with the disease or disorder as a causal agent of the disease or disorder, a regulator of a causal agent of the disease or disorder, a biomarker of the disease or disorder, a sign of a level of severity of the disease or disorder, and/or a sign of a prognosis of the disease or disorder.

The disclosure provides a method of treating a disease or disorder, comprising administering to a subject a therapeutically-effective amount of a CAR of the disclosure, a nucleic acid of the disclosure, a vector of the disclosure, a cell of any one of the disclosure, or a pharmaceutical composition of the disclosure, wherein a severity of a sign or symptom of the disease or disorder is decreased, thereby treating the disease or disorder. In some embodiments, the disease or disorder comprises a cancer. In some embodiments, the CAR selectively targets an antigen or an epitope associated with the disease or disorder as a causal agent of the disease or disorder, a regulator of a causal agent of the disease or disorder, a biomarker of the disease or disorder, a sign of a level of severity of the disease or disorder, and/or a sign of a prognosis of the disease or disorder.

The disclosure provides a method of preventing a disease or disorder, comprising administering to a subject a therapeutically-effective amount of a CAR of the disclosure, a nucleic acid of the disclosure, a vector of the disclosure, a cell of any one of the disclosure, or a pharmaceutical composition of the disclosure, wherein an onset or a relapse of a sign or symptom of the disease or disorder is delayed or inhibited, thereby preventing the disease or disorder. In some embodiments, the disease or disorder comprises a cancer. In some embodiments, the CAR selectively targets an antigen or an epitope associated with the disease or disorder as a causal agent of the disease or disorder, a regulator of a causal agent of the disease or disorder, a biomarker of the disease or disorder, a sign of a level of severity of the disease or disorder, and/or a sign of a prognosis of the disease or disorder.

### BRIEF DESCRIPTION OF DRAWINGS

FIG. 1 is a schematic diagram of an exemplary CAR of the disclosure that, in some embodiments, includes an extracellular element (including, in some embodiments, a binding element and a spacer element), a transmembrane element, and intracellular element (optionally, comprising a costimulatory element and one or more signaling element(s)).

FIG. 2 is a schematic diagram showing an exemplary spatial relationship between an epitope on the surface of a target cell and binding element on the surface of a CAR T cell, including an illustration of an exemplary measurement of the cell-to-cell distance.

FIG. 3 is a schematic diagram of an exemplary CAR construct of the disclosure showing, in some embodiments, the organization of the different components.

FIG. 4 is a schematic diagram of an exemplary HA-specific CAR construct of the disclosure.

FIG. 5 is a schematic diagram of an exemplary HA-epitope construct showing an exemplary organization of different elements of the HA-epitope construct.

FIG. 6 is a schematic diagram of an exemplary HA-epitope construct of the disclosure.

FIG. 7A is a plot showing the percentage of transduced target cells measured by expression of the transduction marker GFP on live A549-NLR cells.

FIG. 7B is a plot showing the percentage of HA tag surface expression on transduced GFP+ cells. The data show that the expressed epitope is detectable on the A549-NLR target cells.

FIG. 7C is a plot showing the median fluorescence intensity (MFI) for bound anti-HA antibodies on live, transduced cells (GRP+ cells).

FIG. 8A and FIG. 8B are plots showing the percentage of transduced cells measured by surface expression of an EGFR-derived transduction marker on live primary T cells in Donor 1 and Donor 2, respectively.

FIG. 8C and FIG. 8D are plots showing the median fluorescence intensity (MFI) as a measure of transduction efficiency for bound HA-Fc protein on live, transduced cells (EGFRt+ cells) in Donor 1 and Donor 2, respectively.

FIGS. 9A, 9B, 9C, and 9D are a series of plots illustrating the effects of indicated spacers on anti HA scFv-induced cytotoxicity in the presence of NucLight Red-labeled A549 target cells expressing a membrane proximal HA target epitope (A549-HA target 1) in Donor 1.

FIGS. 10A, 10B, 10C, and 10D are a series of plots illustrating the effects of indicated spacers on anti HA scFv-induced cytotoxicity in the presence of NucLight Red-labeled A549 target cells expressing a membrane proximal HA target epitope (A549-HA target 1) in Donor 2.

FIGS. 11A, 11B, 11C, and 11D are a series of plots illustrating the effects of indicated spacers on anti HA scFv-induced cytotoxicity in the presence of NucLight Red-labeled A549 target cells expressing a HA target epitope at an intermediate membrane distance (A549-HA target 6) in Donor 1.

FIGS. 12A, 12B, 12C, and 12D are a series of plots illustrating the effects of indicated spacers on anti HA scFv-induced cytotoxicity in the presence of NucLight Red-labeled A549 target cells expressing a HA target epitope at an intermediate membrane distance (A549-HA target 6) in Donor 2.

FIGS. 13A, 13B, 13C, and 13D are a series of plots illustrating the effects of indicated spacers on anti HA scFv-induced cytotoxicity in the presence of NucLight Red-labeled A549 target cells expressing a membrane distal HA target epitope (A549-HA target 16) in Donor 1.

FIGS. 14A, 14B, 14C, and 14D are a series of plots illustrating the effects of indicated spacers on anti HA scFv-induced cytotoxicity in the presence of NucLight Red-labeled A549 target cells expressing a membrane distal HA target epitope (A549-HA target 16) in Donor 2.

FIGS. 15A, 15B, 15C, and 15D are a series of plots illustrating the effects of indicated spacers on anti HA scFv-induced cytotoxicity in the presence of NucLight Red-labeled A549 target cells without HA target epitope.

FIG. 16A and FIG. 16B are plots showing IFN-γ secretion profiles on HA CAR-T cells derived from Donor 1 and Donor 2, respectively, expressing indicated spacers in the absence of target cells.

FIG. 17A is a series of scatter plots summarizing the effects of systematically varying HA epitope membrane distances on optimal anti-HA CAR spacers lengths on target-dependent cytokine secretion and killing AUC for Donor 1 CAR T cells co-cultured with a A549-HA target cell (i.e., target cells 1 through 4, see Table 2).

FIG. 17B is a series of scatter plots summarizing the effects of systematically varying HA epitope membrane distances on optimal anti-HA CAR spacers lengths on target-dependent cytokine secretion and killing AUC for Donor 2 CAR T cells co-cultured with a A549-HA target cell (i.e., target cells 1 through 4, see Table 2).

FIG. 18A is a series of scatter plots summarizing the effects of systematically varying HA epitope membrane distances on optimal anti-HA CAR spacers lengths on target-dependent cytokine secretion and killing AUC for Donor 1 CAR T cells co-cultured with a A549-HA target cell (i.e., target cells 5 through 8, see Table 2).

FIG. 18B is a series of scatter plots summarizing the effects of systematically varying HA epitope membrane distances on optimal anti-HA CAR spacers lengths on target-dependent cytokine secretion and killing AUC for Donor 2 CAR T cells co-cultured with a A549-HA target cell (i.e., target cells 5 through 8, see Table 2).

FIG. 19A is a series of scatter plots summarizing the effects of systematically varying HA epitope membrane distances on optimal anti-HA CAR spacers lengths on target-dependent cytokine secretion and killing AUC for Donor 1 CAR T cells co-cultured with a A549-HA target cell (i.e., target cells 9 through 12, see Table 2).

FIG. 19B are a series of scatter plots summarizing the effects of systematically varying HA epitope membrane distances on optimal anti-HA CAR spacers lengths on target-dependent cytokine secretion and killing AUC for Donor 2 CAR T cells co-cultured with a A549-HA target cell (i.e., target cells 9 through 12, see Table 2).

FIG. 20A are a series of scatter plots summarizing the effects of systematically varying HA epitope membrane distances on optimal anti-HA CAR spacers lengths on target-dependent cytokine secretion and killing AUC for Donor 1 CAR T cells co-cultured with a A549-HA target cell (i.e., target cells 13 through 16, see Table 2).

FIG. 20B are a series of scatter plots summarizing the effects of systematically varying HA epitope membrane distances on optimal anti-HA CAR spacers lengths on target-dependent cytokine secretion and killing AUC for Donor 2 CAR T cells co-cultured with a A549-HA target cell (i.e., target cells 13 through 16, see Table 2).

FIG. 21A is a pair of superimposed scatter plots summarizing the effects of cell-to-cell distance on AUC calculated from IncuCyte killing curves and IFN-γ secretion in Donor 1 and Donor 2, respectively, measured 24 hours post co-culturing.

FIG. 21B is a pair of superimposed scatter plots summarizing the effects of cell-to-cell distance on IFN-7 secretion in Donor 1 and Donor 2, respectively.

FIG. 22A is a series of plots showing IFN-γ secretion levels for CAR spacers CD27_1 and spacer 1 expressed in Donor 1 and Donor 2 paired with each target cell line (i.e., targets 1 through 16).

FIG. 22B is a series of plots showing IFN-7 secretion levels for CAR spacers ICOS and spacer 29 expressed in Donor 1 and Donor 2 paired with each target cell line (i.e., targets 1 through 16).

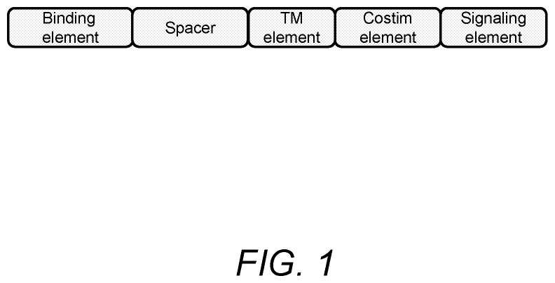

### DETAILED DESCRIPTION

The disclosure demonstrates that the optimal cell-to-cell distance between the CAR-expressing cell and the antigen-expressing cell of the immune synapse is greater than anticipated prior to the instant disclosure. Functional cell-to-cell distances between the TCR-expressing cell and the antigen-expressing cell of an immune synapse have an upper boundary of about 15 nm. In contrast, the instant disclosure demonstrates that, optimally, a CAR stimulates the cell expressing it when the cell-to-cell distance between the CAR-expressing cell and the antigen-expressing cell of the immune synapse is between about 17 nm to about 25 nm. Within this range, the instant disclosure demonstrates that, optimally, a CAR stimulates the cell expressing it when the cell-to-cell distance between the CAR-expressing cell and the antigen-expressing cell of the immune synapse is about 20 nm. The cell-to-cell distance of the immune synapse with a CAR-expressing cell is a variable which achieves superior activation or stimulation through the CAR. In some embodiments, a spacer of the CARs of the disclosure that achieves the cell-to-cell distance between the CAR-expressing cell and the antigen-expressing cell of the immune synapse of between about 17 nm to about 25 nm, results in the superior result of an optimally activated CAR-expressing cell of the disclosure. In some embodiments, a spacer of the CARs of the disclosure that achieves the cell-to-cell distance between the CAR-expressing cell and the antigen-expressing cell of the immune synapse of about 20 nm, results in the superior result of an optimally activated CAR-expressing cell of the disclosure.

The disclosure provides a physical length of each spacer sequence described by the disclosure. The physical length of a spacer of a given sequence may refer to the length of the spacer protein when expressed by a cell of the disclosure. In some embodiments, given two spacers, each having the same number of amino acids may have distinct physical lengths due to the identity of the amino acids with the sequences of each spacer, the secondary structure of the spacer, and/or the interactions of the expressed and folded protein with its environment, including additional proteins fused thereto. In some embodiments, the physical length of a spacer of a given sequence when expressed by a cell of the disclosure is about or is approximately the physical length of the spacer when expressed by any cell, under conditions suitable for transcribing nucleic acid sequences, under conditions suitable for translating nucleic acids to amino acids, under conditions suitable for folding amino acid sequences into a functional protein, and/or under conditions suitable for covalent or non-covalent bond formation.

The disclosure provides Chimeric Antigen Receptors (CARs) comprising (a) an extracellular element comprising (i) a binding element that specifically binds to a target and (ii) a spacer sequence; (b) a transmembrane element; and (c) an intracellular element; wherein, when the CAR is expressed on the surface of a cell, a cell-to-cell distance between a CAR expressing cell and a target expressing cell is between about 17 nm to about 25 nm, inclusive of the endpoints. In some embodiments, the cell-to-cell distance between a CAR expressing cell and a target expressing cell is about 20 nm. In some embodiments, the cell-to-cell distance between a CAR expressing cell and a target expressing cell is 20 nm. The discovery of the spacer sequence length, for example, as a variable that results in superior CAR signaling efficacy when expressed on the surface of a cell and when contacted with an antigen to which it is capable of specifically binding, is unexpected. Based on previous studies of the immune synapse, including presentation of a target by a major histocompatibility complex (MHC) and signaling through a T-Cell receptor (TCR), one of ordinary skill in the art would readily recognize that a cell-to-cell distance at that synapse of 14-15 nm provides efficacious TCR signaling. However, the disclosure provides empirical evidence to show that, when the immune synapse is formed by an MHC presenting an antigen to a CAR, the optimal cell-to-cell distance at the immune synapse is between about 17 nm and about 25 nm, the peak efficacy residing at about 20 nm. The CARs of the disclosure are designed to comprise spacer sequences to achieve a cell-to-cell distance at this MHC-CAR synapse of between about 17 nm and about 25 nm, and preferably, of about 20 nm.

The tables below demonstrate relationships between target distance from target-expressing cells, spacer sequence length and cell-to-cell distance.

TABLE A

| | | Spacer Sequences | |
|---|---|---|---|
| Target or | Optimal CAR | (Spacer Name or No., | |
| epitope | Spacer | See Table 1) of the | |
| distance | sequence | disclosure suitable for | |
| (Angstroms | length | achieving a combined | Spacer Sequence |
| (Å)) | (Å) | length of 170-230 Å | (SEQ ID NO:) |
| 30-40 | 140-190 | 6 | 6 |
| | | CD28 | 36 |
| | | Long(CH2CH3) | 43 |
| | | 41BB_2 | 31 |
| | | 7 | 7 |
| 50-60 | 120-170 | CD28 | 36 |
| | | Long(CH2CH3) | 43 |
| | | 41BB_2 | 31 |
| | | 7 | 7 |
| 60-70 | 110-160 | CD28 | 36 |
| | | Long(CH2CH3) | 43 |
| | | 41BB_2 | 31 |
| | | 7 | 7 |
| | | 16 | 14 |
| | | 29 | 27 |
| | | ICOS | 40 |
| 70-80 | 100-150 | Long(CH2CH3) | 43 |
| | | 41BB_2 | 31 |
| | | 7 | 7 |
| | | 16 | 14 |
| | | 29 | 27 |
| | | ICOS | 40 |
| | | 41BB_3 | 32 |
| | | Intermediate | 42 |
| 80-90 | 90-140 | Long(CH2CH3) | 43 |
| | | 41BB_2 | 31 |
| | | 7 | 7 |
| | | 16 | 14 |
| | | 29 | 27 |
| | | ICOS | 40 |
| | | 41BB_3 | 32 |
| | | Intermediate | 42 |
| | | 8 | 8 |
| 90-100 | 80-130 | 16 | 14 |
| | | 29 | 27 |
| | | ICOS | 40 |
| | | 41BB_3 | 32 |
| | | Intermediate | 42 |
| | | 8 | 8 |
| | | 25 | 23 |
| 100-110 | 70-120 | 16 | 14 |
| | | 29 | 27 |
| | | ICOS | 40 |
| | | 41BB_3 | 32 |
| | | Intermediate | 42 |
| | | 8 | 8 |
| | | 25 | 23 |
| | | 9 | 9 |
| | | 10 | 10 |
| | | 26 | 24 |
| | | 11 | 11 |
| | | 3 | 3 |
| | | 20 | 18 |
| 110-120 | 60-110 | 41BB_3 | 32 |
| | | Intermediate | 42 |
| | | 8 | 8 |
| | | 25 | 23 |
| | | 9 | 9 |
| | | 10 | 10 |
| | | 26 | 24 |
| | | 11 | 11 |
| | | 3 | 3 |
| | | 20 | 18 |
| | | 27 | 25 |
| | | Dap10 | 38 |
| | | 23 | 21 |
| | | 31 | 29 |
| | | short_nodisulf | 37 |
| | | GS_short_nodisulf | 39 |
| 120-130 | 50-100 | 41BB_3 | 32 |
| | | Intermediate | 42 |
| | | 8 | 8 |
| | | 25 | 23 |
| | | 9 | 9 |
| | | 10 | 10 |
| | | 26 | 24 |
| | | 11 | 11 |
| | | 3 | 3 |
| | | 20 | 18 |
| | | 27 | 25 |
| | | Dap10 | 38 |
| | | 23 | 21 |
| | | 31 | 29 |
| | | short_nodisulf | 37 |
| | | GS_short_nodisulf | 39 |
| | | 2 | 2 |
| | | 17 | 15 |
| | | 18 | 16 |
| | | 28 | 26 |
| | | 30 | 28 |
| | | 1 | 1 |
| | | 19 | 17 |
| 130-140 | 40-90 | 25 | 23 |
| | | 9 | 9 |
| | | 10 | 10 |
| | | 26 | 24 |
| | | 11 | 11 |
| | | 3 | 3 |
| | | 20 | 18 |
| | | 27 | 25 |
| | | Dap10 | 38 |
| | | 23 | 21 |
| | | 31 | 29 |
| | | short_nodisulf | 37 |
| | | GS_short_nodisulf | 39 |
| | | 2 | 2 |
| | | 17 | 15 |
| | | 18 | 16 |
| | | 28 | 26 |
| | | 30 | 28 |
| | | 1 | 1 |
| | | 19 | 17 |
| | | 13 | 12 |
| | | 15 | 13 |
| | | 21 | 19 |
| | | 22 | 20 |
| | | Short(IgG4 Hinge) | 44 |
| 140-150 | 30-80 | 9 | 9 |
| | | 10 | 10 |
| | | 26 | 24 |
| | | 11 | 11 |
| | | 3 | 3 |
| | | 20 | 18 |
| | | 27 | 25 |
| | | Dap10 | 38 |
| | | 23 | 21 |
| | | 31 | 29 |
| | | short_nodisulf | 37 |
| | | GS_short_nodisulf | 39 |
| | | 2 | 2 |
| | | 17 | 15 |
| | | 18 | 16 |
| | | 28 | 26 |
| | | 30 | 28 |
| | | 1 | 1 |
| | | 19 | 17 |
| | | 13 | 12 |
| | | 15 | 13 |
| | | 21 | 19 |
| | | 22 | 20 |
| | | Short(IgG4 Hinge) | 44 |
| 150-160 | 20-70 | 27 | 25 |
| | | Dap10 | 38 |
| | | 23 | 21 |
| | | 31 | 29 |
| | | short_nodisulf | 37 |
| | | GS_short_nodisulf | 39 |
| | | 2 | 2 |
| | | 17 | 15 |
| | | 18 | 16 |
| | | 28 | 26 |
| | | 30 | 28 |
| | | 1 | 1 |
| | | 19 | 17 |
| | | 13 | 12 |
| | | 15 | 13 |
| | | 21 | 19 |
| | | 22 | 20 |
| | | Short(IgG4 Hinge) | 44 |
| 160-170 | 10-60 | 2 | 2 |
| | | 17 | 15 |
| | | 18 | 16 |
| | | 28 | 26 |
| | | 30 | 28 |
| | | 1 | 1 |
| | | 19 | 17 |
| | | 13 | 12 |
| | | 15 | 13 |
| | | 21 | 19 |
| | | 22 | 20 |
| | | Short(IgG4 Hinge) | 44 |

TABLE B

| | | Spacer Sequences | |
|---|---|---|---|
| | Optimal CAR | (Spacer Name or No., | |
| Target or | Spacer | See Table 1) of the | |
| epitope | sequence | disclosure suitable for | |
| distance | length | achieving a combined | Spacer Sequence |
| (Å) | (Å) | length of 180-220 Å | (SEQ ID NO:) |
| 30-40 | 150-180 | CD28 | 36 |
| 50-60 | 130-160 | CD28 | 36 |
| | | Long(CH2CH3) | 43 |
| | | 41BB_2 | 31 |
| | | 7 | 7 |
| 60-70 | 120-150 | Long(CH2CH3) | 43 |
| | | 41BB_2 | 31 |
| | | 7 | 7 |
| 70-80 | 110-140 | Long(CH2CH3) | 43 |
| | | 41BB_2 | 31 |
| | | 7 | 7 |
| | | 16 | 14 |
| | | 29 | 27 |
| | | ICOS | 40 |
| 80-90 | 100-130 | 16 | 14 |
| | | 29 | 27 |
| | | ICOS | 40 |
| | | 41BB_3 | 32 |
| | | Intermediate | 42 |
| 90-100 | 90-120 | 16 | 14 |
| | | 29 | 27 |
| | | ICOS | 40 |
| | | 41BB_3 | 32 |
| | | Intermediate | 42 |
| | | 8 | 8 |
| | | 25 | 23 |
| 100-110 | 80-110 | 41BB_3 | 32 |
| | | Intermediate | 42 |
| | | 8 | 8 |
| | | 25 | 23 |
| 110-120 | 70-100 | 8 | 8 |
| | | 25 | 23 |
| | | 9 | 9 |
| | | 10 | 10 |
| | | 26 | 24 |
| | | 11 | 11 |
| | | 3 | 3 |
| | | 20 | 18 |
| 120-130 | 60-90 | 25 | 23 |
| | | 9 | 9 |
| | | 10 | 10 |
| | | 26 | 24 |
| | | 11 | 11 |
| | | 3 | 3 |
| | | 20 | 18 |
| | | 27 | 25 |
| | | Dap10 | 38 |
| | | 23 | 21 |
| | | 31 | 29 |
| | | short_nodisulf | 37 |
| | | GS_short_nodisulf | 39 |
| 130-140 | 50-80 | 9 | 9 |
| | | 10 | 10 |
| | | 26 | 24 |
| | | 11 | 11 |
| | | 3 | 3 |
| | | 20 | 18 |
| | | 27 | 25 |
| | | Dap10 | 38 |
| | | 23 | 21 |
| | | 31 | 29 |
| | | short_nodisulf | 37 |
| | | GS_short_nodisulf | 39 |
| | | 2 | 2 |
| | | 17 | 15 |
| | | 18 | 16 |
| | | 28 | 26 |
| | | 30 | 28 |
| | | 19 | 17 |
| 140-150 | 40-70 | 27 | 25 |
| | | Dap10 | 38 |
| | | 23 | 21 |
| | | 31 | 29 |
| | | short_nodisulf | 37 |
| | | GS_short_nodisulf | 39 |
| | | 2 | 2 |
| | | 17 | 15 |
| | | 18 | 16 |
| | | 28 | 26 |
| | | 30 | 28 |
| | | 19 | 17 |
| | | 13 | 12 |
| | | 15 | 13 |
| | | 21 | 19 |
| | | 22 | 20 |
| | | Short(IgG4 Hinge) | 44 |
| 150-160 | 30-60 | 2 | 2 |
| | | 17 | 15 |
| | | 18 | 16 |
| | | 28 | 26 |
| | | 30 | 28 |
| | | 19 | 17 |
| | | 13 | 12 |
| | | 15 | 13 |
| | | 21 | 19 |
| | | 22 | 20 |
| | | Short(IgG4 Hinge) | 44 |
| 160-170 | 20-50 | 13 | 12 |
| | | 15 | 13 |
| | | 21 | 19 |
| | | 22 | 20 |
| | | Short(IgG4 Hinge) | 44 |

TABLE C

| | | Spacer Sequences | |
|---|---|---|---|
| | Optimal CAR | (Spacer Name or No., | |
| Target or | Spacer | See Table 1) of the | |
| epitope | sequence | disclosure suitable for | |
| distance | length | achieving a combined | Spacer Sequence |
| (Å) | (Å) | length of 170-250 Å | (SEQ ID NO:) |
| 30-40 | 140-210 | 5 | 5 |
| | | 41BB_1 | 30 |
| | | CD8a | 33 |
| | | OX40 | 41 |
| | | 6 | 6 |
| | | CD28 | 36 |
| 50-60 | 120-190 | 6 | 6 |
| | | CD28 | 36 |
| | | Long(CH2CH3) | 43 |
| | | 41BB_2 | 31 |
| | | 7 | 7 |
| 60-70 | 110-180 | CD28 | 36 |
| | | Long(CH2CH3) | 43 |
| | | 41BB_2 | 31 |
| | | 7 | 7 |
| | | 16 | 14 |
| | | 29 | 27 |
| | | ICOS | 40 |
| 70-80 | 100-170 | CD28 | 36 |
| | | Long(CH2CH3) | 43 |
| | | 41BB_2 | 31 |
| | | 7 | 7 |
| | | 16 | 14 |
| | | 29 | 27 |
| | | ICOS | 40 |
| | | 41BB_3 | 32 |
| | | Intermediate | 42 |
| 80-90 | 90-160 | CD28 | 36 |
| | | Long(CH2CH3) | 43 |
| | | 41BB_2 | 31 |
| | | 7 | 7 |
| | | 16 | 14 |
| | | 29 | 27 |
| | | ICOS | 40 |
| | | 41BB_3 | 32 |
| | | Intermediate | 42 |
| | | 8 | 8 |
| 90-100 | 80-150 | Long(CH2CH3) | 43 |
| | | 41BB_2 | 31 |
| | | 7 | 7 |
| | | 16 | 14 |
| | | 29 | 27 |
| | | ICOS | 40 |
| | | 41BB_3 | 32 |
| | | Intermediate | 42 |
| | | 8 | 8 |
| | | 25 | 23 |
| 100-110 | 70-140 | 16 | 14 |
| | | 29 | 27 |
| | | ICOS | 40 |
| | | 41BB_3 | 32 |
| | | Intermediate | 42 |
| | | 8 | 8 |
| | | 25 | 23 |
| | | 9 | 9 |
| | | 10 | 10 |
| | | 26 | 24 |
| | | 11 | 11 |
| | | 3 | 3 |
| | | 20 | 18 |
| 110-120 | 60-130 | 16 | 14 |
| | | 29 | 27 |
| | | ICOS | 40 |
| | | 41BB_3 | 32 |
| | | Intermediate | 42 |
| | | 8 | 8 |
| | | 25 | 23 |
| | | 9 | 9 |
| | | 10 | 10 |
| | | 26 | 24 |
| | | 11 | 11 |
| | | 3 | 3 |
| | | 20 | 18 |
| | | 27 | 25 |
| | | Dap10 | 38 |
| | | 23 | 21 |
| | | 31 | 29 |
| | | short_nodisulf | 37 |
| | | GS_short_nodisulf | 39 |
| 120-130 | 50-120 | 41BB_1 | 30 |
| | | CD8a | 33 |
| | | OX40 | 41 |
| | | 6 | 6 |
| | | CD28 | 36 |
| | | Long(CH2CH3) | 43 |
| | | 41BB_2 | 31 |
| | | 7 | 7 |
| | | 16 | 14 |
| | | 29 | 27 |
| | | ICOS | 40 |
| | | 41BB_3 | 32 |
| | | Intermediate | 42 |
| | | 8 | 8 |
| | | 25 | 23 |
| | | 9 | 9 |
| | | 10 | 10 |
| | | 26 | 24 |
| | | 11 | 11 |
| | | 3 | 3 |
| | | 20 | 18 |
| | | 27 | 25 |
| | | Dap10 | 38 |
| | | 23 | 21 |
| | | 31 | 29 |
| | | short_nodisulf | 37 |
| | | GS_short_nodisulf | 39 |
| | | 2 | 2 |
| | | 17 | 15 |
| | | 18 | 16 |
| | | 28 | 26 |
| | | 30 | 28 |
| | | 1 | 1 |
| | | 19 | 17 |
| 130-140 | 40-110 | 41BB_3 | 32 |
| | | Intermediate | 42 |
| | | 8 | 8 |
| | | 25 | 23 |
| | | 9 | 9 |
| | | 10 | 10 |
| | | 26 | 24 |
| | | 11 | 11 |
| | | 3 | 3 |
| | | 20 | 18 |
| | | 27 | 25 |
| | | Dap10 | 38 |
| | | 23 | 21 |
| | | 31 | 29 |
| | | short_nodisulf | 37 |
| | | GS_short_nodisulf | 39 |
| | | 2 | 2 |
| | | 17 | 15 |
| | | 18 | 16 |
| | | 28 | 26 |
| | | 30 | 28 |
| | | 1 | 1 |
| | | 19 | 17 |
| | | 13 | 12 |
| | | 15 | 13 |
| | | 21 | 19 |
| | | 22 | 20 |
| | | Short(IgG4 Hinge) | 44 |
| 140-150 | 30-100 | 8 | 8 |
| | | 25 | 23 |
| | | 9 | 9 |
| | | 10 | 10 |
| | | 26 | 24 |
| | | 11 | 11 |
| | | 3 | 3 |
| | | 20 | 18 |
| | | 27 | 25 |
| | | Dap10 | 38 |
| | | 23 | 21 |
| | | 31 | 29 |
| | | short_nodisulf | 37 |
| | | GS_short_nodisulf | 39 |
| | | 2 | 2 |
| | | 17 | 15 |
| | | 18 | 16 |
| | | 28 | 26 |
| | | 30 | 28 |
| | | 1 | 1 |
| | | 19 | 17 |
| | | 13 | 12 |
| | | 15 | 13 |
| | | 21 | 19 |
| | | 22 | 20 |
| | | Short(IgG4 Hinge) | 44 |
| 150-160 | 20-90 | 25 | 23 |
| | | 9 | 9 |
| | | 10 | 10 |
| | | 26 | 24 |
| | | 11 | 11 |
| | | 3 | 3 |
| | | 20 | 18 |
| | | 27 | 25 |
| | | Dap10 | 38 |
| | | 23 | 21 |
| | | 31 | 29 |
| | | short_nodisulf | 37 |
| | | GS_short_nodisulf | 39 |
| | | 2 | 2 |
| | | 17 | 15 |
| | | 18 | 16 |
| | | 28 | 26 |
| | | 30 | 28 |
| | | 1 | 1 |
| | | 19 | 17 |
| | | 13 | 12 |
| | | 15 | 13 |
| | | 21 | 19 |
| | | 22 | 20 |
| | | Short(IgG4 Hinge) | 44 |
| 160-170 | 10-80 | 9 | 9 |
| | | 10 | 10 |
| | | 26 | 24 |
| | | 11 | 11 |
| | | 3 | 3 |
| | | 20 | 18 |
| | | 27 | 25 |
| | | Dap10 | 38 |
| | | 23 | 21 |
| | | 31 | 29 |
| | | short_nodisulf | 37 |
| | | GS_short_nodisulf | 39 |
| | | 2 | 2 |
| | | 17 | 15 |
| | | 18 | 16 |
| | | 28 | 26 |
| | | 30 | 28 |
| | | 1 | 1 |
| | | 19 | 17 |
| | | 13 | 12 |
| | | 15 | 13 |
| | | 21 | 19 |
| | | 22 | 20 |
| | | Short(IgG4 Hinge) | 44 |
| 180-190 | 1-60 | 2 | 2 |
| | | 17 | 15 |
| | | 18 | 16 |
| | | 28 | 26 |
| | | 30 | 28 |
| | | 1 | 1 |
| | | 19 | 17 |
| | | 13 | 12 |
| | | 15 | 13 |
| | | 21 | 19 |
| | | 22 | 20 |
| | | Short(IgG4 Hinge) | 44 |
| 190-200 | 1-50 | 13 | 12 |
| | | 15 | 13 |
| | | 21 | 19 |
| | | 22 | 20 |
| | | Short(IgG4 Hinge) | 44 |

The disclosure provides Chimeric Antigen Receptors (CARs) comprising (a) an extracellular element comprising (i) a binding element that specifically binds to a target and (ii) a spacer sequence; (b) a transmembrane element; and (c) an intracellular element; wherein, when the CAR is expressed on the surface of a cell, a cell-to-cell distance between a CAR expressing cell and a target expressing cell is between about 17 nm to about 25 nm, inclusive of the endpoints. The design of the CARs of the disclosure is based on the studies provided here, the results of which demonstrate that when a CAR includes a spacer of a given length sufficient to achieve a cell-to-cell distance of between about 17 nm to about 25 nm, the CAR activity is superior when compared to a CAR having a spacer that does not provide this cell-to-cell distance or when compared to a CAR lacking a spacer sequence. The result-effective variable of spacer length sufficient to achieve the cell-to-cell distance within the range of between about 17 nm to about 25 nm has not been elucidated before this disclosure. Moreover, as CARs of the disclosure may contain binding elements having various lengths and formats (e.g., scFv, monoclonal antibody, protein scaffold) and targets may have varying size as well as distance from the cells that express them, the disclosure provides detailed guidance for selecting a spacer sequence to generate a CAR that, for each binding element and target, achieves a cell-to-cell distance of between about 17 nm to about 25 nm.

### Chimeric Antigen Receptors (CARs)

The disclosure provides a CAR comprising (a) an extracellular element comprising (i) a binding element that specifically binds to a target and (ii) a spacer sequence; (b) a transmembrane element; and (c) an intracellular element; wherein, when the CAR is expressed on the surface of a cell, a cell-to-cell distance between a CAR expressing cell and a target expressing cell is between about 17 nm to about 25 nm, inclusive of the endpoints. In some embodiments, the cell-to-cell distance between a CAR expressing cell and a target expressing cell is about 20 nm.

In some embodiments of the CARs of the disclosure, the CAR may comprise one or more human sequences. In some embodiments, the CAR may comprise one or more sequences isolated or derived from a human sequence. In some embodiments, the one or more sequences derived from a human sequence may have at least 50%, 55%, 60%, 65%, 70%, 75%, 80%, 85%, 90%, 95%, 97%, 99% or any percentage in between of identity to a naturally-occurring human sequence. In some embodiments, the one or more sequences derived from a human sequence may have one or more synthetic amino acids. In some embodiments, the CAR may comprise one or more sequences modified to covalently or non-covalently bind a synthetic moiety, including a synthetic amino acid. In some embodiments, the CAR may comprise one or more sequences that do not occur together in a naturally occurring sequence or that do not function in the same endogenous pathway, thereby rendering the CAR a chimeric receptor. In some embodiments, the CAR may comprise one or more sequences isolated or derived from a non-human sequence. In some embodiments, the one or more sequences derived from a non-human sequence may have at least 50%, 55%, 60%, 65%, 70%, 75%, 80%, 85%, 90%, 95%, 97%, 99% or any percentage in between of identity to a naturally-occurring non-human sequence.

In some embodiments of the CARs of the disclosure, including those comprising (a) an extracellular element comprising (1) a binding element that specifically binds to a target and (2) a spacer sequence; (b) a transmembrane element; and (c) an intracellular element; wherein, when the CAR is expressed on the surface of a cell, a cell-to-cell distance between a CAR expressing cell and a target expressing cell is between about 17 nm to about 25 nm, inclusive of the endpoints, the binding element comprises an antigen recognition domain. In some embodiments, the binding element comprises a first antigen recognition domain and a second antigen recognition domain. In some embodiments, the binding element or the antigen recognition domain comprises an antibody (including, but not limited to, a monoclonal antibody), a protein scaffold, an antibody mimetic, or an antigen binding sequence thereof. In some embodiments, the binding element or the antigen recognition domain comprises one or more of a monoclonal antibody, a single domain antibody, a domain antibody, a VH or VHH, a monobody or a single-chain variable fragment (scFv).

In some embodiments of the CARs of the disclosure, including those comprising (a) an extracellular element comprising (1) a binding element that specifically binds to a target and (2) a spacer sequence; (b) a transmembrane element; and (c) an intracellular element; wherein, when the CAR is expressed on the surface of a cell, a cell-to-cell distance between a CAR expressing cell and a target expressing cell is between about 17 nm to about 25 nm, inclusive of the endpoints, the binding element comprises an antigen recognition domain. In some embodiments, the transmembrane element comprises a sequence isolated or derived from a sequence of a CD4 protein. In some embodiments, the transmembrane element comprises a sequence isolated or derived from a sequence of a CD8 protein. In some embodiments, the transmembrane element comprises a sequence isolated or derived from a sequence of a CD28 protein. In some embodiments, a CD28 protein comprises an amino acid sequence of

| | |
|---|---|
| 1 | MLRLLLALNL FPSIQVIGNK ILVKQSPMLV AYDNAVNLSC KYSYNLFSRE FRASLHKGLD |
| 61 | SAVEVCVVYG NYSQQLQVYS KTGFNCDGKL GNESVTFYLQ NLYVNQTDIY FCKIEVMYPP |
| 121 | PYLDNEKSNG TIIHVKGKHL CPSPLFPGPS KPFWVLVVVG GVLACYSLLV TVAFIIFWVR |
| 181 | SKRSRLLHSD YMNMTPRRPG PTRKHYQPYA PPRDFAAYRS |

(SEQ ID NO: 45 and UniProtKB Accession No. P10747-1). In some embodiments, the transmembrane element comprises a sequence isolated or derived from a sequence of a CD28 protein. In some embodiments, a CD28 protein comprises an amino acid sequence of

| | |
|---|---|
| 1 | MLRILLALNL FPSIQVTGKH LCPSPLFPGP SKPFWVLVVV GGVLACYSLL VTVAFIIFWV |
| 61 | RSKRSRLLHS DYMNMTPRRP GPTRKHYQPY APPRDFAAYR S |

(SEQ ID NO: 46 and UniProtKB Accession No. P10747-2). In some embodiments, a CD28 protein comprises an amino acid sequence of

| | |
|---|---|
| 1 | MLRLLLALNL FPSIQVTGNK ILVKQSPMLV AYDNAVNLSY NEKSNGTIIH VKGKHLCPSP |
| 61 | LFPGPSKPFW VLVVVGGVLA CYSLLVTVAF IIFWVRSKRS RLLHSDYMNM TPRRPGPTRK |
| 121 | HYQPYAPPRD FAAYRS |

(SEQ ID NO: 47 and UniProtKB Accession No. P10747-3). In some embodiments, a CD28 protein comprises an amino acid sequence of

| | |
|---|---|
| 1 | MLRLLLALNL FPSIQVTGNK ILVKQSPMLV AYDNAVNLSW KHLCPSPLFP GPSKPFWVLV |
| 61 | VVGGVLACYS LLVTVAFIIF WVRSKRSRLL HSDYMNMTPR RPGPTRKHYQ PYAPPRDFAA |
| 121 | YRS |

(SEQ ID NO: 48 and UniProtKB Accession No. P10747-4). In some embodiments, a CD28 protein comprises an amino acid sequence of

| | |
|---|---|
| 1 | MLRLLLALNL FPSIQVTGNK ILVKQSPMLV AYDNAVNLSY NEKSNGTIIH VKGEE |

(SEQ ID NO: 49 and UniProtKB Accession No. P10747-5). In some embodiments, a CD28 protein comprises an amino acid sequence of

| | |
|---|---|
| 1 | MLRLLLALNL FPSIQVIGNK ILVKQSPMLV AYDNAVNLSY NEKSNGTIIH VKGKHLCPSP |
| 61 | LFPGPSKPYA PPRDFAAYRS |

(SEQ ID NO: 50 and UniProtKB Accession No. P10747-6). In some embodiments, a CD28 protein comprises an amino acid sequence of

| | |
|---|---|
| 1 | MPCGLSALIM CPKGMVAVVV AVDDGDSQAL AGNKILVKQS PMLVAYDNAV NLSCKYSYNL |
| 61 | FSREFRASLH KGLDSAVEVC VVYGNYSQQL QVYSKTGFNC DGKLGNESVT FYLQNLYVNQ |
| 121 | TDIYFCKIEV MYPPPYLDNE KSNGTIIHVK GKHLCPSPLF PGPSKPFWVL VVVGGVLACY |
| 181 | SLLVTVAFII FWVRSKRSRL LHSDYMNMTP RRPGPTRKHY QPYAPPRDFA AYRS |

(SEQ ID NO: 51 and UniProtKB Accession No. P10747-7). In some embodiments, a CD28 protein comprises an amino acid sequence of FWVLVVVGGVLACYSLLVTVAFIIFWV (SEQ ID NO: 52).

In some embodiments of the CARs of the disclosure, including those comprising (a) an extracellular element comprising (1) a binding element that specifically binds to a target and (2) a spacer sequence; (b) a transmembrane element; and (c) an intracellular element; wherein, when the CAR is expressed on the surface of a cell, a cell-to-cell distance between a CAR expressing cell and a target expressing cell is between about 17 nm to about 25 nm, inclusive of the endpoints, the intracellular element comprises a costimulatory element comprising a sequence isolated or derived from one or more of a TNF receptor protein, an Immunoglobulin-like proteins, a cytokine receptor, an integrin, a signaling lymphocytic activation molecule (SLAM) protein, and an activating natural killer cell receptor. In some embodiments, the intracellular element comprises a costimulatory element comprising a sequence isolated or derived from one or more of CD27, CD28, 4-1BB (CD137), OX40, GITR, CD30, CD40, ICOS, BAFFR, HVEM, lymphocyte function-associated antigen-1 (LFA-1), CD2, CD7, LIGHT, NKG2C, SLAMFE7, NKp80, CD160, B7-H3, and MyD88.

In some embodiments of the CARs of the disclosure, including those comprising (a) an extracellular element comprising (1) a binding element that specifically binds to a target and (2) a spacer sequence; (b) a transmembrane element; and (c) an intracellular element; wherein, when the CAR is expressed on the surface of a cell, a cell-to-cell distance between a CAR expressing cell and a target expressing cell is between about 17 nm to about 25 nm, inclusive of the endpoints, the intracellular element comprises a 4-1BB costimulatory element. In some embodiments, the 4-1BB costimulatory element comprises a sequence of

| | |
|---|---|
| 1 | MGNSCYNIVA TLLLVLNFER TRSLQDPCSN CPAGTFCDNN RNQICSPCPP NSFSSAGGQR |
| 61 | TCDICRQCKG VFRTRKECSS TSNAECDCTP GFHCLGAGCS MCEQDCKQGQ ELTKKGCKDC |
| 121 | CFGTFNDQKR GICRPWINCS LDGKSVLVNG TKERDVVCGP SPADLSPGAS SVTPPAPARE |
| 181 | PGHSPQIISF FLALTSTALL FLLFFLTLRF SVVKRGRKKL LYIFKQPFMR PVQTTQEEDG |
| 241 | CSCRFPEEEE GGCEL |

(SEQ ID NO: 53 and UniProtKB Accession No. Q07011-1). In some embodiments, the 4-1BB costimulatory element comprises a sequence of

(SEQ ID NO: 54)
KRGRKKLLYIFKQPFMRPVQTTQEEDGCSCRFPEEEEGGCEL.

In some embodiments of the CARs of the disclosure, including those comprising (a) an extracellular element comprising (1) a binding element that specifically binds to a target and (2) a spacer sequence; (b) a transmembrane element; and (c) an intracellular element; wherein, when the CAR is expressed on the surface of a cell, a cell-to-cell distance between a CAR expressing cell and a target expressing cell is between about 17 nm to about 25 nm, inclusive of the endpoints, the intracellular element comprises a CD28 costimulatory element. In some embodiments, the CD28 costimulatory element comprises a sequence of SEQ ID NOs: 45-52. In some embodiments, the CD28 costimulatory element comprises a sequence having at least 50%, 55%, 60%, 65%, 70%, 75%, 80%, 85%, 90%, 95%, 97%, 99%, or any percentage in between, identity to SEQ ID NOs: 45-52.

In some embodiments of the CARs of the disclosure, including those comprising (a) an extracellular element comprising (1) a binding element that specifically binds to a target and (2) a spacer sequence; (b) a transmembrane element; and (c) an intracellular element; wherein, when the CAR is expressed on the surface of a cell, a cell-to-cell distance between a CAR expressing cell and a target expressing cell is between about 17 nm to about 25 nm, inclusive of the endpoints, the intracellular element comprises a CD27 costimulatory element. In some embodiments, the CD27 costimulatory element comprises a sequence of

| | |
|---|---|
| 1 | MARPHPWWLC VLGTLVGLSA TPAPKSCPER HYWAQGKLCC QMCEPGTFLV KDCDQHRKAA |
| 61 | QCDPCIPGVS FSPDHHTRPH CESCRHCNSG LLVRNCTITA NAECACRNGW QCRDKECTEC |
| 121 | DPLPNPSLTA RSSQALSPHP QPTHLPYVSE MLEARTAGHM QTLADFRQLP ARTLSTHWPP |
| 181 | QRSLCSSDFI RILVIFSGMF LVFTLAGALF LHQRRKYRSN KGESPVEPAE PCHYSCPREE |
| 241 | EGSTIPIQED YRKPEPACSP |

(SEQ ID NO: 55 and UniProt Accession No. P26842-1).

In some embodiments of the CARs of the disclosure, including those comprising (a) an extracellular element comprising (1) a binding element that specifically binds to a target and (2) a spacer sequence; (b) a transmembrane element; and (c) an intracellular element; wherein, when the CAR is expressed on the surface of a cell, a cell-to-cell distance between a CAR expressing cell and a target expressing cell is between about 17 nm to about 25 nm, inclusive of the endpoints, the intracellular element comprises a CD3 zeta signaling element. In some embodiments, the CD3 zeta signaling element comprises a sequence of

| | |
|---|---|
| 1 | MKWKALFTAA ILQAQLPITE AQSFGLLDPK LCYLLDGILF IYGVILTALF LRVKFSRSAD |
| 61 | APAYQQGQNQ LYNELNLGRR EEYDVLDKRR GRDPEMGGKP QRRKNPQEGL YNELQKDKMA |
| 121 | EAYSEIGMKG ERRRGKGHDG LYQGLSTATK DTYDALHMQA LPPR |

(SEQ ID NO: 76 and UniProtKB Accession No. P20963-1). In some embodiments, the CD3 zeta signaling element comprises a sequence of

| | |
|---|---|
| 1 | MKWKALFTAA ILQAQLPITE AQSFGLLDPK LCYLLDGILF IYGVILTALF LRVKFSRSAD |
| 61 | APAYQQGQNQ LYNELNLGRR EEYDVLDKRR GRDPEMGGKP RRKNPQEGLY NELQKDKMA E |
| 121 | AYSEIGMKGE RRRGKGHDGL YQGLSTATKD TYDALHMQAL PPR |

(SEQ ID NO: 56 and UniProtKB Accession No. P20963-3). In some embodiments, the CD3 zeta signaling element comprises a sequence of

(SEQ ID NO: 57)
RVKFSRSADAPAYQQGQNQLYNELNLGRREEYDVLDKRRGRDPEMGGKPR
RKNPQEGLYNELQKDKMAEAYSEIGMKGERRRGKGHDGLYQGLSTATKDT
YDALHMQALPPR.

In some embodiments of the CARs of the disclosure, including those comprising (a) an extracellular element comprising (1) a binding element that specifically binds to a target and (2) a spacer sequence; (b) a transmembrane element; and (c) an intracellular element; wherein, when the CAR is expressed on the surface of a cell, a cell-to-cell distance between a CAR expressing cell and a target expressing cell is between about 17 nm to about 25 nm, inclusive of the endpoints, the intracellular element comprises a 4-1BB costimulatory element of the disclosure and a CD3 zeta signaling element of the disclosure. In some embodiments, the intracellular element further comprises a transduction marker. In some embodiments, the transduction marker comprises an EGFR-derived marker. In some embodiments, the EGFR-derived marker comprises a truncated EGFR (EGFRt). In some embodiments, the intracellular element further comprises a sequence comprising a self-cleaving peptide. In some embodiment, the sequence comprising a self-cleaving peptide is positioned between the transduction marker and either a 4-1BB costimulatory element of the disclosure or a CD3 zeta signaling element of the disclosure.

In some embodiments of the CARs of the disclosure, including those comprising (a) an extracellular element comprising (1) a binding element that specifically binds to a target and (2) a spacer sequence; (b) a transmembrane element; and (c) an intracellular element; wherein, when the CAR is expressed on the surface of a cell, a cell-to-cell distance between a CAR expressing cell and a target expressing cell is between about 17 nm to about 25 nm, inclusive of the endpoints, the binding element or the antigen recognition domain specifically binds to a target, or any portion thereof, isolated or derived from an extracellular antigen. In some embodiments, the target, or any portion thereof, isolated or derived from an extracellular antigen is present or expressed on a surface of a cell.

In some embodiments of the CARs of the disclosure, including those comprising (a) an extracellular element comprising (1) a binding element that specifically binds to a target and (2) a spacer sequence; (b) a transmembrane element; and (c) an intracellular element; wherein, when the CAR is expressed on the surface of a cell, a cell-to-cell distance between a CAR expressing cell and a target expressing cell is between about 17 nm to about 25 nm, inclusive of the endpoints, the target expressing cell is in vivo, in vitro, or ex vivo. In some embodiments, the target expressing cell comprises one or more modifications. In some embodiments, the target expressing cell is genetically modified.

In some embodiments of the CARs of the disclosure, the CAR comprises a chimeric polypeptide that includes: (i) a chimeric fusion protein including an extracellular antigen recognition element, (ii) a transmembrane element, (iii) an intracellular signaling element including a functional signaling element derived from a stimulatory molecule, and (iv) a spacer linking the extracellular antigen recognition element to a transmembrane element.

In some embodiments of the CARs of the disclosure, the CAR comprises a chimeric polypeptide that includes: (i) a chimeric fusion protein including an extracellular antigen recognition element, (ii) a transmembrane element, (iii) an intracellular signaling element including a functional signaling element derived from a costimulatory molecule and a functional signaling element derived from a stimulatory molecule, and (iv) a spacer linking the extracellular antigen recognition element to a transmembrane element.

In some embodiments of the CARs of the disclosure, the CAR comprises a chimeric polypeptide that includes: (i) an extracellular antigen recognition element, (ii) a transmembrane element, an intracellular signaling element including two functional signaling elements derived from one or more co-stimulatory molecules and a functional signaling element derived from a stimulatory molecule, and (iii) spacer linking the extracellular antigen recognition element to a transmembrane element.

In some embodiments of the CARs of the disclosure, the CAR comprises a chimeric (i) an extracellular antigen recognition element, (ii) a transmembrane element, (iii) an intracellular signaling element including at least two functional signaling elements derived from one or more co-stimulatory molecules and a functional signaling element derived from a stimulatory molecule, and (iv) a spacer linking the extracellular antigen recognition element to a transmembrane element.

In some embodiments of the CARs of the disclosure, the CAR comprises an optional leader sequence. In some embodiments, the leader sequence is positioned at the amino-terminus (N-term) of the CAR. In some embodiments, the CAR comprises a fusion protein. In some embodiments, the leader sequence is positioned at the N-terminus of the CAR fusion protein. In some embodiments, the leader sequence is positioned at the N-terminus of the extracellular element of the CAR. In some embodiments, a cleavable sequence is positioned between either the leader sequence and the CAR or the leader sequence and the extracellular element of the CAR. In some embodiments, the cleavable sequence comprises a self-cleaving peptide. In some embodiments, the leader sequence is cleaved from the CAR during cellular processing. In some embodiments, cleavage of the leader sequence induces localization of the CAR to the cellular membrane.

In some embodiments of the CARs of the disclosure, the CAR comprises a fusion protein. In some embodiments, the fusion protein comprises a leader sequence. In some embodiments, a cleavable sequence is positioned between the leader sequence and the CAR. In some embodiments, the cleavable sequence comprises a self-cleaving peptide. In some embodiments, the leader sequence is cleaved from the CAR during cellular processing. In some embodiments, cleavage of the leader sequence induces localization of the CAR to the cellular membrane.

In some embodiments of the CARs of the disclosure, including those comprising (a) an extracellular element comprising (1) a binding element that specifically binds to a target and (2) a spacer sequence; (b) a transmembrane element; and (c) an intracellular element, the binding element that specifically binds to a target, and wherein, when the target sequence is expressed on the surface of a target cell, the target sequence is between about 3 nm and about 4 nm from the surface of the target cell; and the spacer sequence has a length of between about 14 nm and about 19 nm; wherein, when the CAR is expressed on the surface of a cell, a cell-to-cell distance between a CAR expressing cell and a target expressing cell is between about 17 nm to 23 nm, inclusive of the endpoints. In some embodiments of the CARs of the disclosure, including those comprising (a) an extracellular element comprising (1) a binding element that specifically binds to a target and (2) a spacer sequence; (b) a transmembrane element; and (c) an intracellular element, the binding element that specifically binds to a target, and wherein, when the target sequence is expressed on the surface of a target cell, the target sequence is between about 3 nm and about 4 nm from the surface of the target cell; the spacer sequence has a length of between about 14 nm and about 19 nm; and the spacer sequence comprises a sequence of any one of SEQ ID NO: 6, 36, 43, 31, or 7; wherein, when the CAR is expressed on the surface of a cell, a cell-to-cell distance between a CAR expressing cell and a target expressing cell is between about 17 nm to 23 nm, inclusive of the endpoints.

In some embodiments of the CARs of the disclosure, including those comprising (a) an extracellular element comprising (1) a binding element that specifically binds to a target and (2) a spacer sequence; (b) a transmembrane element; and (c) an intracellular element, the binding element that specifically binds to a target, and wherein, when the target sequence is expressed on the surface of a target cell, the target sequence is between about 5 nm and about 6 nm from the surface of the target cell and the spacer sequence has a length of between about 12 nm and about 17 nm; wherein, when the CAR is expressed on the surface of a cell, a cell-to-cell distance between a CAR expressing cell and a target expressing cell is between about 17 nm to 23 nm, inclusive of the endpoints. In some embodiments of the CARs of the disclosure, including those comprising (a) an extracellular element comprising (1) a binding element that specifically binds to a target and (2) a spacer sequence; (b) a transmembrane element; and (c) an intracellular element, the binding element that specifically binds to a target, and wherein, when the target sequence is expressed on the surface of a target cell, the target sequence is between about 5 nm and about 6 nm from the surface of the target cell; the spacer sequence has a length of between about 12 nm and about 17 nm; and the spacer sequence comprises a sequence of any one of SEQ ID NO: 36, 43, 31, or 7; wherein, when the CAR is expressed on the surface of a cell, a cell-to-cell distance between a CAR expressing cell and a target expressing cell is between about 17 nm to 23 nm, inclusive of the endpoints.

In some embodiments of the CARs of the disclosure, including those comprising (a) an extracellular element comprising (1) a binding element that specifically binds to a target and (2) a spacer sequence; (b) a transmembrane element; and (c) an intracellular element, the binding element that specifically binds to a target, and wherein, when the target sequence is expressed on the surface of a target cell, the target sequence is between about 6 nm and about 7 nm from the surface of the target cell; and the spacer sequence has a length of between about 11 nm and about 16 nm; wherein, when the CAR is expressed on the surface of a cell, a cell-to-cell distance between a CAR expressing cell and a target expressing cell is between about 17 nm to 23 nm, inclusive of the endpoints. In some embodiments of the CARs of the disclosure, including those comprising (a) an extracellular element comprising (1) a binding element that specifically binds to a target and (2) a spacer sequence; (b) a transmembrane element; and (c) an intracellular element, the binding element that specifically binds to a target, and wherein, when the target sequence is expressed on the surface of a target cell, the target sequence is between about 6 nm and about 7 nm from the surface of the target cell; the sp acer sequence has a length of between about 11 nm and about 16 nm; and the spacer sequence comprises a sequence of any one of SEQ ID NO: 36, 43, 31, 7, 14, 27, or 40; wherein, when the CAR is expressed on the surface of a cell, a cell-to-cell distance between a CAR expressing cell and a target expressing cell is between about 17 nm to 23 nm, inclusive of the endpoints.

In some embodiments of the CARs of the disclosure, including those comprising (a) an extracellular element comprising (1) a binding element that specifically binds to a target and (2) a spacer sequence; (b) a transmembrane element; and (c) an intracellular element, the binding element that specifically binds to a target, and wherein, when the target sequence is expressed on the surface of a target cell, the target sequence is between about 7 nm and about 8 nm from the surface of the target cell; and the spacer sequence has a length of between about 10 nm and about 15 nm; wherein, when the CAR is expressed on the surface of a cell, a cell-to-cell distance between a CAR expressing cell and a target expressing cell is between about 17 nm to 23 nm, inclusive of the endpoints. In some embodiments of the CARs of the disclosure, including those comprising (a) an extracellular element comprising (1) a binding element that specifically binds to a target and (2) a spacer sequence; (b) a transmembrane element; and (c) an intracellular element, the binding element that specifically binds to a target, and wherein, when the target sequence is expressed on the surface of a target cell, the target sequence is between about 7 nm and about 8 nm from the surface of the target cell; the spacer sequence has a length of between about 10 nm and about 15 nm; and the spacer sequence comprises a sequence of any one of SEQ ID NO: 43, 31, 7, 14, 27, 40, 32 or 42; wherein, when the CAR is expressed on the surface of a cell, a cell-to-cell distance between a CAR expressing cell and a target expressing cell is between about 17 nm to 23 nm, inclusive of the endpoints.

In some embodiments of the CARs of the disclosure, including those comprising (a) an extracellular element comprising (1) a binding element that specifically binds to a target and (2) a spacer sequence; (b) a transmembrane element; and (c) an intracellular element, the binding element that specifically binds to a target, and wherein, when the target sequence is expressed on the surface of a target cell, the target sequence is between about 8 nm and about 9 nm from the surface of the target cell; and the spacer sequence has a length of between about 9 nm and about 14 nm; wherein, when the CAR is expressed on the surface of a cell, a cell-to-cell distance between a CAR expressing cell and a target expressing cell is between about 17 nm to 23 nm, inclusive of the endpoints. In some embodiments of the CARs of the disclosure, including those comprising (a) an extracellular element comprising (1) a binding element that specifically binds to a target and (2) a spacer sequence; (b) a transmembrane element; and (c) an intracellular element, the binding element that specifically binds to a target, and wherein, when the target sequence is expressed on the surface of a target cell, the target sequence is between about 8 nm and about 9 nm from the surface of the target cell; the spacer sequence has a length of between about 9 nm and about 14 nm; and the spacer sequence comprises a sequence of any one of SEQ ID NO: 43, 31, 7, 14, 27, 40, 32, 42 or 8; wherein, when the CAR is expressed on the surface of a cell, a cell-to-cell distance between a CAR expressing cell and a target expressing cell is between about 17 nm to 23 nm, inclusive of the endpoints.

In some embodiments of the CARs ofthe disclosure, including those comprising (a) an extracellular element comprising (1) a binding element that specifically binds to a target and (2) a spacer sequence; (b) a transmembrane element; and (c) an intracellular element, the binding element that specifically binds to a target, and wherein, when the target sequence is expressed on the surface of a target cell, the target sequence is between about 9 nm and about 10 nm from the surface of the target cell; and the spacer sequence has a length of between about 8 nm and about 13 nm; wherein, when the CAR is expressed on the surface of a cell, a cell-to-cell distance between a CAR expressing cell and a target expressing cell is between about 17 nm to 23 nm, inclusive of the endpoints. In some embodiments of the CARs of the disclosure, including those comprising (a) an extracellular element comprising (1) a binding element that specifically binds to a target and (2) a spacer sequence; (b) a transmembrane element; and (c) an intracellular element, the binding element that specifically binds to a target, and wherein, when the target sequence is expressed on the surface of a target cell, the target sequence is between about 9 nm and about 10 nm from the surface of the target cell; the spacer sequence has a length of between about 8 nm and about 13 nm; and the spacer sequence comprises a sequence of any one of SEQ ID NO: 14, 27, 40, 32, 42, 8 or 23; wherein, when the CAR is expressed on the surface of a cell, a cell-to-cell distance between a CAR expressing cell and a target expressing cell is between about 17 nm to 23 nm, inclusive of the endpoints.

In some embodiments of the CARs of the disclosure, including those comprising (a) an extracellular element comprising (1) a binding element that specifically binds to a target and (2) a spacer sequence; (b) a transmembrane element; and (c) an intracellular element, the binding element that specifically binds to a target, and wherein, when the target sequence is expressed on the surface of a target cell, the target sequence is between about 10 nm and about 11 nm from the surface of the target cell; and the spacer sequence has a length of between about 7 nm and about 12 nm; wherein, when the CAR is expressed on the surface of a cell, a cell-to-cell distance between a CAR expressing cell and a target expressing cell is between about 17 nm to 23 nm, inclusive of the endpoints. In some embodiments of the CARs of the disclosure, including those comprising (a) an extracellular element comprising (1) a binding element that specifically binds to a target and (2) a spacer sequence; (b) a transmembrane element; and (c) an intracellular element, the binding element that specifically binds to a target, and wherein, when the target sequence is expressed on the surface of a target cell, the target sequence is between about 10 nm and about 11 nm from the surface of the target cell; the spacer sequence has a length of between about 7 nm and about 12 nm; and the spacer sequence comprises a sequence of any one of SEQ ID NO: 14, 27, 40, 32, 42, 8, 23, 9, 10, 24, 11, 3, or 18; wherein, when the CAR is expressed on the surface of a cell, a cell-to-cell distance between a CAR expressing cell and a target expressing cell is between about 17 nm to 23 nm, inclusive of the endpoints.

In some embodiments of the CARs of the disclosure, including those comprising (a) an extracellular element comprising (1) a binding element that specifically binds to a target and (2) a spacer sequence; (b) a transmembrane element; and (c) an intracellular element, the binding element that specifically binds to a target, and wherein, when the target sequence is expressed on the surface of a target cell, the target sequence is between about 11 nm and about 12 nm from the surface of the target cell; the spacer sequence has a length of between about 6 nm and about 11 nm; and the spacer sequence comprises a sequence of any one of SEQ ID NO: 32, 42, 8, 23, 9, 10, 24, 11, 3, 18, 25, 38, 21, 29, 37 or 39; wherein, when the CAR is expressed on the surface of a cell, a cell-to-cell distance between a CAR expressing cell and a target expressing cell is between about 17 nm to 23 nm, inclusive of the endpoints. In some embodiments of the CARs of the disclosure, including those comprising (a) an extracellular element comprising (1) a binding element that specifically binds to a target and (2) a spacer sequence; (b) a transmembrane element; and (c) an intracellular element, the binding element that specifically binds to a target, and wherein, when the target sequence is expressed on the surface of a target cell, the target sequence is between about 11 nm and ab out 12 nm from the surface of the target cell; the spacer sequence has a length of between about 6 nm and about 11 nm; and the spacer sequence comprises a sequence of any one of SEQ ID NO: 32, 42, 8, 23, 9, 10, 24, 11, 3, 18, 25, 38, 21, 29, 37 or 39; wherein, when the CAR is expressed on the surface of a cell, a cell-to-cell distance between a CAR expressing cell and a target expressing cell is between about 17 nm to 23 nm, inclusive of the endpoints.

In some embodiments of the CARs of the disclosure, including those comprising (a) an extracellular element comprising (1) a binding element that specifically binds to a target and (2) a spacer sequence; (b) a transmembrane element; and (c) an intracellular element, the binding element that specifically binds to a target, and wherein, when the target sequence is expressed on the surface of a target cell, the target sequence is between about 12 nm and about 13 nm from the surface of the target cell; and the spacer sequence has a length of between about 5 nm and about 10 nm; wherein, when the CAR is expressed on the surface of a cell, a cell-to-cell distance between a CAR expressing cell and a target expressing cell is between about 17 nm to 23 nm, inclusive of the endpoints. In some embodiments of the CARs of the disclosure, including those comprising (a) an extracellular element comprising (1) a binding element that specifically binds to a target and (2) a spacer sequence; (b) a transmembrane element; and (c) an intracellular element, the binding element that specifically binds to a target, and wherein, when the target sequence is expressed on the surface of a target cell, the target sequence is between about 12 nm and about 13 nm from the surface of the target cell; the spacer sequence has a length of between about 5 nm and about 10 nm; and the spacer sequence comprises a sequence of any one of SEQ ID NO: 32, 42, 8, 23, 9, 10, 24, 11, 3, 18, 25, 38, 21, 29, 37, 39, 2, 15, 16, 26, 28, 1, or 17; wherein, when the CAR is expressed on the surface of a cell, a cell-to-cell distance between a CAR expressing cell and a target expressing cell is between about 17 nm to 23 nm, inclusive of the endpoints.

In some embodiments of the CARs of the disclosure, including those comprising (a) an extracellular element comprising (1) a binding element that specifically binds to a target and (2) a spacer sequence; (b) a transmembrane element; and (c) an intracellular element, the binding element that specifically binds to a target, and wherein, when the target sequence is expressed on the surface of a target cell, the target sequence is between about 13 nm and about 14 nm from the surface of the target cell; and the spacer sequence has a length of between about 4 nm and about 9 nm; wherein, when the CAR is expressed on the surface of a cell, a cell-to-cell distance between a CAR expressing cell and a target expressing cell is between about 17 nm to 23 nm, inclusive of the endpoints. In some embodiments of the CARs of the disclosure, including those comprising (a) an extracellular element comprising (1) a binding element that specifically binds to a target and (2) a spacer sequence; (b) a transmembrane element; and (c) an intracellular element, the binding element that specifically binds to a target, and wherein, when the target sequence is expressed on the surface of a target cell, the target sequence is between about 13 nm and about 14 nm from the surface of the target cell; the spacer sequence has a length of between about 4 nm and about 9 nm; and the spacer sequence comprises a sequence of any one of SEQ ID NO: 23, 9, 10, 24, 11, 3, 18, 25, 38, 21, 29, 37, 39, 2, 15, 16, 26, 28, 1, 17, 12, 13, 19, 20, or 44; wherein, when the CAR is expressed on the surface of a cell, a cell-to-cell distance between a CAR expressing cell and a target expressing cell is between about 17 nm to 23 nm, inclusive of the endpoints.

In some embodiments of the CARs of the disclosure, including those comprising (a) an extracellular element comprising (1) a binding element that specifically binds to a target and (2) a spacer sequence; (b) a transmembrane element; and (c) an intracellular element, the binding element that specifically binds to a target, and wherein, when the target sequence is expressed on the surface of a target cell, the target sequence is between about 14 nm and about 15 nm from the surface of the target cell; and the spacer sequence has a length of between about 3 nm and about 8 nm; wherein, when the CAR is expressed on the surface of a cell, a cell-to-cell distance between a CAR expressing cell and a target expressing cell is between about 17 nm to 23 nm, inclusive of the endpoints. In some embodiments of the CARs of the disclosure, including those comprising (a) an extracellular element comprising (1) a binding element that specifically binds to a target and (2) a spacer sequence; (b) a transmembrane element; and (c) an intracellular element, the binding element that specifically binds to a target, and wherein, when the target sequence is expressed on the surface of a target cell, the target sequence is between about 14 nm and about 15 nm from the surface of the target cell; the spacer sequence has a length of between about 3 nm and about 8 nm; and the spacer sequence comprises a sequence of any one of SEQ ID NO: 9, 10, 24, 11, 3, 18, 25, 38, 21, 29, 37, 39, 2, 15, 16, 26, 28, 1, 17, 12, 13, 19, 20, or 44; wherein, when the CAR is expressed on the surface of a cell, a cell-to-cell distance between a CAR expressing cell and a target expressing cell is between about 17 nm to 23 nm, inclusive of the endpoints.

In some embodiments of the CARs of the disclosure, including those comprising (a) an extracellular element comprising (1) a binding element that specifically binds to a target and (2) a spacer sequence; (b) a transmembrane element; and (c) an intracellular element, the binding element that specifically binds to a target, and wherein, when the target sequence is expressed on the surface of a target cell, the target sequence is between about 15 nm and about 16 nm from the surface of the target cell; and the spacer sequence has a length of between about 2 nm and about 7 nm; wherein, when the CAR is expressed on the surface of a cell, a cell-to-cell distance between a CAR expressing cell and a target expressing cell is between about 17 nm to 23 nm, inclusive of the endpoints. In some embodiments of the CARs of the disclosure, including those comprising (a) an extracellular element comprising (1) a binding element that specifically binds to a target and (2) a spacer sequence; (b) a transmembrane element; and (c) an intracellular element, the binding element that specifically binds to a target, and wherein, when the target sequence is expressed on the surface of a target cell, the target sequence is between about 15 nm and about 16 nm from the surface of the target cell; the spacer sequence has a length of between about 2 nm and about 7 nm; and the spacer sequence comprises a sequence of any one of SEQ ID NO: 25, 38, 21, 29, 37, 39, 2, 15, 16, 26, 28, 1, 17, 12, 13, 19, 20, or 44; wherein, when the CAR is expressed on the surface of a cell, a cell-to-cell distance between a CAR expressing cell and a target expressing cell is between about 17 nm to 23 nm, inclusive of the endpoints.

In some embodiments of the CARs of the disclosure, including those comprising (a) an extracellular element comprising (1) a binding element that specifically binds to a target and (2) a spacer sequence; (b) a transmembrane element; and (c) an intracellular element, the binding element that specifically binds to a target, and wherein, when the target sequence is expressed on the surface of a target cell, the target sequence is between about 16 nm and about 17 nm from the surface of the target cell; and the spacer sequence has a length of between about 1 nm and about 6 nm; wherein, when the CAR is expressed on the surface of a cell, a cell-to-cell distance between a CAR expressing cell and a target expressing cell is between about 17 nm to 23 nm, inclusive of the endpoints. In some embodiments of the CARs of the disclosure, including those comprising (a) an extracellular element comprising (1) a binding element that specifically binds to a target and (2) a spacer sequence; (b) a transmembrane element; and (c) an intracellular element, the binding element that specifically binds to a target, and wherein, when the target sequence is expressed on the surface of a target cell, the target sequence is between about 16 nm and about 17 nm from the surface of the target cell; the spacer sequence has a length of between about 1 nm and about 6 nm; and the spacer sequence comprises a sequence of any one of SEQ ID NO: 2, 15, 16, 26, 28, 1, 17, 12, 13, 19, 20, or 44; wherein, when the CAR is expressed on the surface of a cell, a cell-to-cell distance between a CAR expressing cell and a target expressing cell is between about 17 nm to 23 nm, inclusive of the endpoints.

In some embodiments of the CARs of the disclosure, including those comprising (a) an extracellular element comprising (1) a binding element that specifically binds to a target and (2) a spacer sequence; (b) a transmembrane element; and (c) an intracellular element, the binding element that specifically binds to a target, and wherein, when the target sequence is expressed on the surface of a target cell, the target sequence is between about 3 nm and about 4 nm from the surface of the target cell; and the spacer sequence has a length of between about 15 nm and about 18 nm; wherein, when the CAR is expressed on the surface of a cell, a cell-to-cell distance between a CAR expressing cell and a target expressing cell is between about 18 nm to 22 nm, inclusive of the endpoints. In some embodiments of the CARs of the disclosure, including those comprising (a) an extracellular element comprising (1) a binding element that specifically binds to a target and (2) a spacer sequence; (b) a transmembrane element; and (c) an intracellular element, the binding element that specifically binds to a target, and wherein, when the target sequence is expressed on the surface of a target cell, the target sequence is between about 3 nm and about 4 nm from the surface of the target cell; the spacer sequence has a length of between about 15 nm and about 18 nm; and the spacer sequence comprises a sequence of SEQ ID NO: 36; wherein, when the CAR is expressed on the surface of a cell, a cell-to-cell distance between a CAR expressing cell and a target expressing cell is between about 18 nm to 22 nm, inclusive of the endpoints.

In some embodiments of the CARs of the disclosure, including those comprising (a) an extracellular element comprising (1) a binding element that specifically binds to a target and (2) a spacer sequence; (b) a transmembrane element; and (c) an intracellular element, the binding element that specifically binds to a target, and wherein, when the target sequence is expressed on the surface of a target cell, the target sequence is between about 5 nm and about 6 nm from the surface of the target cell; and the spacer sequence has a length of between about 13 nm and about 16 nm; wherein, when the CAR is expressed on the surface of a cell, a cell-to-cell distance between a CAR expressing cell and a target expressing cell is between about 18 nm to 22 nm, inclusive of the endpoints. In some embodiments of the CARs of the disclosure, including those comprising (a) an extracellular element comprising (1) a binding element that specifically binds to a target and (2) a spacer sequence; (b) a transmembrane element; and (c) an intracellular element, the binding element that specifically binds to a target, and wherein, when the target sequence is expressed on the surface of a target cell, the target sequence is between about 5 nm and about 6 nm from the surface of the target cell; the spacer sequence has a length of between about 13 nm and about 16 nm; and the spacer sequence comprises a sequence of any one of SEQ ID NO: 36, 43, 31, or 7; wherein, when the CAR is expressed on the surface of a cell, a cell-to-cell distance between a CAR expressing cell and a target expressing cell is between about 18 nm to 22 nm, inclusive of the endpoints.

In some embodiments of the CARs of the disclosure, including those comprising (a) an extracellular element comprising (1) a binding element that specifically binds to a target and (2) a spacer sequence; (b) a transmembrane element; and (c) an intracellular element, the binding element that specifically binds to a target, and wherein, when the target sequence is expressed on the surface of a target cell, the target sequence is between about 6 nm and about 7 nm from the surface of the target cell; and the spacer sequence has a length of between about 12 nm and about 15 nm; wherein, when the CAR is expressed on the surface of a cell, a cell-to-cell distance between a CAR expressing cell and a target expressing cell is between about 18 nm to 22 nm, inclusive of the endpoints. In some embodiments of the CARs of the disclosure, including those comprising (a) an extracellular element comprising (1) a binding element that specifically binds to a target and (2) a spacer sequence; (b) a transmembrane element; and (c) an intracellular element, the binding element that specifically binds to a target, and wherein, when the target sequence is expressed on the surface of a target cell, the target sequence is between about 6 nm and about 7 nm from the surface of the target cell; the spacer sequence has a length of between about 12 nm and about 15 nm; and the spacer sequence comprises a sequence of any one of SEQ ID NO: 43, 31, or 7; wherein, when the CAR is expressed on the surface of a cell, a cell-to-cell distance between a CAR expressing cell and a target expressing cell is between about 18 nm to 22 nm, inclusive of the endpoints.

In some embodiments of the CARs of the disclosure, including those comprising (a) an extracellular element comprising (1) a binding element that specifically binds to a target and (2) a spacer sequence; (b) a transmembrane element; and (c) an intracellular element, the binding element that specifically binds to a target, and wherein, when the target sequence is expressed on the surface of a target cell, the target sequence is between about 7 nm and about 8 nm from the surface of the target cell; and the spacer sequence has a length of between about 11 nm and about 14 nm; wherein, when the CAR is expressed on the surface of a cell, a cell-to-cell distance between a CAR expressing cell and a target expressing cell is between about 18 nm to 22 nm, inclusive of the endpoints. In some embodiments of the CARs of the disclosure, including those comprising (a) an extracellular element comprising (1) a binding element that specifically binds to a target and (2) a spacer sequence; (b) a transmembrane element; and (c) an intracellular element, the binding element that specifically binds to a target, and wherein, when the target sequence is expressed on the surface of a target cell, the target sequence is between about 7 nm and about 8 nm from the surface of the target cell; the spacer sequence has a length of between about 11 nm and about 14 nm; and the spacer sequence comprises a sequence of any one of SEQ ID NO: 43, 31, 7, 14, 27, or 40; wherein, when the CAR is expressed on the surface of a cell, a cell-to-cell distance between a CAR expressing cell and a target expressing cell is between about 18 nm to 22 nm, inclusive of the endpoints.

In some embodiments of the CARs of the disclosure, including those comprising (a) an extracellular element comprising (1) a binding element that specifically binds to a target and (2) a spacer sequence; (b) a transmembrane element; and (c) an intracellular element, the binding element that specifically binds to a target, and wherein, when the target sequence is expressed on the surface of a target cell, the target sequence is between about 8 nm and about 9 nm from the surface ofthe target cell; and the spacer sequence has a length of between about 10 nm and about 13 nm; wherein, when the CAR is expressed on the surface of a cell, a cell-to-cell distance between a CAR expressing cell and a target expressing cell is between about 18 nm to 22 nm, inclusive of the endpoints. In some embodiments of the CARs of the disclosure, including those comprising (a) an extracellular element comprising (1) a binding element that specifically binds to a target and (2) a spacer sequence; (b) a transmembrane element; and (c) an intracellular element, the binding element that specifically binds to a target, and wherein, when the target sequence is expressed on the surface of a target cell, the target sequence is between about 8 nm and about 9 nm from the surface of the target cell; the spacer sequence has a length of between about 10 nm and about 13 nm; and the spacer sequence comprises a sequence of any one of SEQ ID NO: 14, 27, 40, 32 or 42; wherein, when the CAR is expressed on the surface of a cell, a cell-to-cell distance between a CAR expressing cell and a target expressing cell is between about 18 nm to 22 nm, inclusive of the endpoints.

In some embodiments of the CARs of the disclosure, including those comprising (a) an extracellular element comprising (1) a binding element that specifically binds to a target and (2) a spacer sequence; (b) a transmembrane element; and (c) an intracellular element, the binding element that specifically binds to a target, and wherein, when the target sequence is expressed on the surface of a target cell, the target sequence is between about 9 nm and about 10 nm from the surface of the target cell; and the spacer sequence has a length of between about 9 nm and about 12 nm; wherein, when the CAR is expressed on the surface of a cell, a cell-to-cell distance between a CAR expressing cell and a target expressing cell is between about 18 nm to 22 nm, inclusive of the endpoints. In some embodiments of the CARs of the disclosure, including those comprising (a) an extracellular element comprising (1) a binding element that specifically binds to a target and (2) a spacer sequence; (b) a transmembrane element; and (c) an intracellular element, the binding element that specifically binds to a target, and wherein, when the target sequence is expressed on the surface of a target cell, the target sequence is between about 9 nm and about 10 nm from the surface of the target cell; the spacer sequence has a length of between about 9 nm and about 12 nm; and the spacer sequence comprises a sequence of any one of SEQ ID NO: 14, 27, 40, 32, 42, 8 or 23; wherein, when the CAR is expressed on the surface of a cell, a cell-to-cell distance between a CAR expressing cell and a target expressing cell is between about 18 nm to 22 nm, inclusive of the endpoints.

In some embodiments of the CARs of the disclosure, including those comprising (a) an extracellular element comprising (1) a binding element that specifically binds to a target and (2) a spacer sequence; (b) a transmembrane element; and (c) an intracellular element, the binding element that specifically binds to a target, and wherein, when the target sequence is expressed on the surface of a target cell, the target sequence is between about 10 nm and about 11 nm from the surface of the target cell; and the spacer sequence has a length of between about 8 nm and about 11 nm; wherein, when the CAR is expressed on the surface of a cell, a cell-to-cell distance between a CAR expressing cell and a target expressing cell is between about 18 nm to 22 nm, inclusive of the endpoints. In some embodiments of the CARs of the disclosure, including those comprising (a) an extracellular element comprising (1) a binding element that specifically binds to a target and (2) a spacer sequence; (b) a transmembrane element; and (c) an intracellular element, the binding element that specifically binds to a target, and wherein, when the target sequence is expressed on the surface of a target cell, the target sequence is between about 10 nm and about 11 nm from the surface of the target cell; the spacer sequence has a length of between about 8 nm and about 11 nm; and the spacer sequence comprises a sequence of any one of SEQ ID NO: 32, 42, 8 or 23; wherein, when the CAR is expressed on the surface of a cell, a cell-to-cell distance between a CAR expressing cell and a target expressing cell is between about 18 nm to 22 nm, inclusive of the endpoints.

In some embodiments of the CARs of the disclosure, including those comprising (a) an extracellular element comprising (1) a binding element that specifically binds to a target and (2) a spacer sequence; (b) a transmembrane element; and (c) an intracellular element, the binding element that specifically binds to a target, and wherein, when the target sequence is expressed on the surface of a target cell, the target sequence is between about 11 nm and about 12 nm from the surface of the target cell; and the spacer sequence has a length of between about 7 nm and about 10 nm; wherein, when the CAR is expressed on the surface of a cell, a cell-to-cell distance between a CAR expressing cell and a target expressing cell is between about 18 nm to 22 nm, inclusive of the endpoints. In some embodiments of the CARs of the disclosure, including those comprising (a) an extracellular element comprising (1) a binding element that specifically binds to a target and (2) a spacer sequence; (b) a transmembrane element; and (c) an intracellular element, the binding element that specifically binds to a target, and wherein, when the target sequence is expressed on the surface of a target cell, the target sequence is between about 11 nm and about 12 nm from the surface of the target cell; the spacer sequence has a length of between about 7 nm and about 10 nm; and the spacer sequence comprises a sequence of any one of SEQ ID NO: 8, 23, 9, 10, 24, 11, 3, or 18; wherein, when the CAR is expressed on the surface of a cell, a cell-to-cell distance between a CAR expressing cell and a target expressing cell is between about 18 nm to 22 nm, inclusive of the endpoints.

In some embodiments of the CARs of the disclosure, including those comprising (a) an extracellular element comprising (1) a binding element that specifically binds to a target and (2) a spacer sequence; (b) a transmembrane element; and (c) an intracellular element, the binding element that specifically binds to a target, and wherein, when the target sequence is expressed on the surface of a target cell, the target sequence is between about 12 nm and about 13 nm from the surface of the target cell; and the spacer sequence has a length of between about 6 nm and about 9 nm; wherein, when the CAR is expressed on the surface of a cell, a cell-to-cell distance between a CAR expressing cell and a target expressing cell is between about 18 nm to 22 nm, inclusive of the endpoints. In some embodiments of the CARs of the disclosure, including those comprising (a) an extracellular element comprising (1) a binding element that specifically binds to a target and (2) a spacer sequence; (b) a transmembrane element; and (c) an intracellular element, the binding element that specifically binds to a target, and wherein, when the target sequence is expressed on the surface of a target cell, the target sequence is between about 12 nm and about 13 nm from the surface of the target cell; the spacer sequence has a length of between about 6 nm and about 9 nm; and the spacer sequence comprises a sequence of any one of SEQ ID NO: 23, 9, 10, 24, 11, 3, 18, 25, 38, 21, 29, 37, or 39; wherein, when the CAR is expressed on the surface of a cell, a cell-to-cell distance between a CAR expressing cell and a target expressing cell is between about 18 nm to 22 nm, inclusive of the endpoints.

In some embodiments of the CARs of the disclosure, including those comprising (a) an extracellular element comprising (1) a binding element that specifically binds to a target and (2) a spacer sequence; (b) a transmembrane element; and (c) an intracellular element, the binding element that specifically binds to a target, and wherein, when the target sequence is expressed on the surface of a target cell, the target sequence is between about 13 nm and about 14 nm from the surface of the target cell; and the spacer sequence has a length of between about 5 nm and about 8 nm; wherein, when the CAR is expressed on the surface of a cell, a cell-to-cell distance between a CAR expressing cell and a target expressing cell is between about 18 nm to 22 nm, inclusive of the endpoints. In some embodiments of the CARs of the disclosure, including those comprising (a) an extracellular element comprising (1) a binding element that specifically binds to a target and (2) a spacer sequence; (b) a transmembrane element; and (c) an intracellular element, the binding element that specifically binds to a target, and wherein, when the target sequence is expressed on the surface of a target cell, the target sequence is between about 13 nm and about 14 nm from the surface of the target cell; the spacer sequence has a length of between about 5 nm and about 8 nm; and the spacer sequence comprises a sequence of any one of SEQ ID NO: 9, 10, 24, 11, 3, 18, 25, 38, 21, 29, 37, 39, 2, 15, 16, 26, 28, or 17; wherein, when the CAR is expressed on the surface of a cell, a cell-to-cell distance between a CAR expressing cell and a target expressing cell is between about 18 nm to 22 nm, inclusive of the endpoints.

In some embodiments of the CARs of the disclosure, including those comprising (a) an extracellular element comprising (1) a binding element that specifically binds to a target and (2) a spacer sequence; (b) a transmembrane element; and (c) an intracellular element, the binding element that specifically binds to a target, and wherein, when the target sequence is expressed on the surface of a target cell, the target sequence is between about 14 nm and about 15 nm from the surface of the target cell; and the spacer sequence has a length of between about 4 nm and about 7 nm; wherein, when the CAR is expressed on the surface of a cell, a cell-to-cell distance between a CAR expressing cell and a target expressing cell is between about 18 nm to 22 nm, inclusive of the endpoints. In some embodiments of the CARs of the disclosure, including those comprising (a) an extracellular element comprising (1) a binding element that specifically binds to a target and (2) a spacer sequence; (b) a transmembrane element; and (c) an intracellular element, the binding element that specifically binds to a target, and wherein, when the target sequence is expressed on the surface of a target cell, the target sequence is between about 14 nm and about 15 nm from the surface of the target cell; the spacer sequence has a length of between about 4 nm and about 7 nm; and the spacer sequence comprises a sequence of any one of SEQ ID NO: 25, 38, 21, 29, 37, 39, 2, 15, 16, 26, 28, 17, 12, 13, 19, 20, or 44; wherein, when the CAR is expressed on the surface of a cell, a cell-to-cell distance between a CAR expressing cell and a target expressing cell is between about 18 nm to 22 nm, inclusive of the endpoints.

In some embodiments of the CARs of the disclosure, including those comprising (a) an extracellular element comprising (1) a binding element that specifically binds to a target and (2) a spacer sequence; (b) a transmembrane element; and (c) an intracellular element, the binding element that specifically binds to a target, and wherein, when the target sequence is expressed on the surface of a target cell, the target sequence is between about 15 nm and about 16 nm from the surface of the target cell; and the spacer sequence has a length of between about 3 nm and about 6 nm; wherein, when the CAR is expressed on the surface of a cell, a cell-to-cell distance between a CAR expressing cell and a target expressing cell is between about 18 nm to 22 nm, inclusive of the endpoints. In some embodiments of the CARs of the disclosure, including those comprising (a) an extracellular element comprising (1) a binding element that specifically binds to a target and (2) a spacer sequence; (b) a transmembrane element; and (c) an intracellular element, the binding element that specifically binds to a target, and wherein, when the target sequence is expressed on the surface of a target cell, the target sequence is between about 15 nm and about 16 nm from the surface of the target cell; the spacer sequence has a length of between about 3 nm and about 6 nm; and the spacer sequence comprises a sequence of any one of SEQ ID NO: 2, 15, 16, 26, 28, 17, 12, 13, 19, 20, or 44; wherein, when the CAR is expressed on the surface of a cell, a cell-to-cell distance between a CAR expressing cell and a target expressing cell is between about 18 nm to 22 nm, inclusive of the endpoints.

In some embodiments of the CARs of the disclosure, including those comprising (a) an extracellular element comprising (1) a binding element that specifically binds to a target and (2) a spacer sequence; (b) a transmembrane element; and (c) an intracellular element, the binding element that specifically binds to a target, and wherein, when the target sequence is expressed on the surface of a target cell, the target sequence is between about 16 nm and about 17 nm from the surface of the target cell; and the spacer sequence has a length of between about 2 nm and about 5 nm; wherein, when the CAR is expressed on the surface of a cell, a cell-to-cell distance between a CAR expressing cell and a target expressing cell is between about 18 nm to 22 nm, inclusive of the endpoints. In some embodiments of the CARs of the disclosure, including those comprising (a) an extracellular element comprising (1) a binding element that specifically binds to a target and (2) a spacer sequence; (b) a transmembrane element; and (c) an intracellular element, the binding element that specifically binds to a target, and wherein, when the target sequence is expressed on the surface of a target cell, the target sequence is between about 16 nm and about 17 nm from the surface of the target cell; the spacer sequence has a length of between about 2 nm and about 5 nm; and the spacer sequence comprises a sequence of any one of SEQ ID NO: 12, 13, 19, 20, or 44; wherein, when the CAR is expressed on the surface of a cell, a cell-to-cell distance between a CAR expressing cell and a target expressing cell is between about 18 nm to 22 nm, inclusive of the endpoints.

### Spacers

The disclosure provides a spacer sequence for each CAR-epitope binding pair to achieve a cell-to-cell distance of between about 17 nm and about 25 nm or between 17 nm and 25 nm. In some embodiments, the disclosure provides a set of spacer sequences for each CAR-epitope binding pair to achieve a cell-to-cell distance of between about 17 nm and about 25 nm or between 17 nm and 25 nm.

The disclosure provides a spacer sequence for each CAR-epitope binding pair to achieve a cell-to-cell distance of between about 17 nm and about 22 nm or between 17 nm and 22 nm. In some embodiments, the disclosure provides a set of spacer sequences for each CAR-epitope binding pair to achieve a cell-to-cell distance of between about 17 nm and about 22 nm or between 17 nm and 22 nm.

The disclosure provides a spacer sequence for each CAR-epitope binding pair to achieve a cell-to-cell distance of about 20 nm or 20 nm. In some embodiments, the disclosure provides a set of spacer sequences for each CAR-epitope binding pair to achieve a cell-to-cell distance of about 20 nm or 20 nm.

In some embodiments, a set of test spacers comprises the first test spacer and the second test spacer. In some embodiments, the set of spacers comprises at least one spacer comprising a sequence of SEQ ID NO: 1-44. In some embodiments, the set of spacers comprises at least 2, 3, 4, 5, 6, 7, 8, 9, 10, 15, 20, 25, 30, 35, 40 or 44 spacers comprising a sequence of SEQ ID NO: 1-44. In some embodiments, the set of spacers comprises each one of the sequences of SEQ ID NO: 1-44.

In some embodiments of the spacer sequences of the disclosure, spacer sequences comprise one or more of distinct sequences, distinct lengths, distinct secondary geometry, distinct tertiary geometry, distinct flexibility, distinct charge profile, distinct hydrophobicity, distinct hydrophilicity, and distinct immunogenicity.

In some embodiments of the spacer sequences ofthe disclosure, spacer sequences comprise distinct lengths.

In some embodiments of the spacer sequences of the disclosure, spacer sequences comprise a length of between about 2 nm to about 20 nm. In some embodiments, spacer sequences comprise a length of between about 3 nm to about 20 nm. In some embodiments, the spacer sequences comprise a length of between about 4 nm to about 20 nm.

In some embodiments of the spacer sequences of the disclosure, spacer sequences comprise a sequence of SEQ ID NO: 1-44. In some embodiments, the spacer sequence comprises a sequence at least 90%, 91%, 92%, 93%, 94%, 95%, 96%, 97%, 98%, or 99% identical to the sequence of SEQ ID NO: 1-44.

In some embodiments of the spacer sequences of the disclosure, a spacer sequence is derived from one or more of a human immunoglobulin (Ig) protein, the hinge region of an immunoglobulin protein, or an extracellular domain of a protein. In some embodiments of the spacer sequences of the disclosure, a spacer sequence is engineered de novo. In some embodiments of the spacer sequences of the disclosure, a spacer sequence comprises less than 50% identity to any known human sequence.

In some embodiments of the spacer sequences of the disclosure, a spacer sequence comprises a linker sequence. In some embodiments, the linker sequence comprises or encodes for a flexible linker. In some embodiments, the linker sequence comprises a Glycine-Serine (GS) linker. In some embodiments, the linker sequence comprises the sequence of GGGSG (SEQ ID NO: 77).

In some embodiments of the spacer sequences of the disclosure, a CAR comprises a linker sequence. In some embodiments, the linker sequence comprises or encodes for a flexible linker. In some embodiments, the linker sequence comprises a Glycine-Serine (GS) linker. In some embodiments, the linker sequence comprises the sequence of GGGSG (SEQ ID NO: 77). In some embodiments, the linker sequence is positioned between the binding element and the spacer sequence.

Table 1 provides exemplary spacer sequences of the disclosure. In some embodiments, CARs of the disclosure comprise one or more spacer sequences of Table 1. In some embodiments, CARs of the disclosure comprise one or more spacer sequences of the disclosure (including those provided in Table 1), wherein the spacer sequence comprises one or more of a deletion, an insertion, a substitution, an inversion, a truncation or a modification. In some embodiments, the spacer sequence comprises a sequence having at least 50%, 55%, 60%, 65%, 70%, 75%, 80%, 85%, 80%, 95%, 97%, 99% identity to one or more of SEQ ID NO: 1-44. In some embodiments, the spacer sequence comprises at least one non-naturally occurring residue. In some embodiments, the substitution comprises a replacement of one first amino acid for a second amino acid, wherein first amino acid and the second amino acid have one or more of polarity, side chain length, or hydrophobicity. For example, in some embodiments, a cysteine (C) is replaced by a serine (S).

TABLE 1
Exemplary Spacer Sequences (amino acid)

| | Linker | | CAR | |
|---|---|---|---|---|
| | Se- | | Spacer | |
| Spa- | quence | | Se- | |
| cer | (SEQ | | quence | Å |
| No. | ID | | SEQ | (with |
| or | NO: | | ID | link- |
| Name | 77) | Spacer Sequence | NO: | er) |
| 1 | GGGSG | KPCPPCKCP | 1 | 50.4 |
| 2 | GGGSG | KPCPPCKCPAP | 2 | 57.6 |
| 3 | GGGSG | GPTIKPCPPCKCPAP | 3 | 72 |
| 4 | GGGSG | ELKTPLGDTTHTCPRCPEPKSCD | 4 | 248.4 |
| | | TPPPCPRCPEPKSCDTPPPCPRCP | | |
| | | EPKSCDTPPPCPRCPAP | | |
| 5 | GGGSG | CPRCPEPKSCDTPPPCPRCPEPKS | 5 | 205.2 |
| | | CDTPPPCPRCPEPKSCDTPPPCPR | | |
| | | CPAP | | |
| 6 | GGGSG | EPKSCDTPPPCPRCPEPKSCDTPP | 6 | 187.2 |
| | | PCPRCPEPKSCDTPPPCPRCPAP | | |
| 7 | GGGSG | EPKSCDTPPPCPRCPEPKSCDTPP | 7 | 133.2 |
| | | PCPRCPAP | | |
| 8 | GGGSG | CPRCPEPKSCDTPPPCPRCPAP | 8 | 97.2 |
| 9 | GGGSG | EPKSCDTPPPCPRCPAP | 9 | 79.2 |
| 10 | GGGSG | EPKSCDKTHTCPPCPAP | 10 | 79.2 |
| 11 | GGGSG | PKSCDKTHTCPPCPAP | 11 | 75.6 |
| 13 | GGGSG | PCPRCPAP | 12 | 46.8 |
| 15 | GGGSG | PPKPKDT | 13 | 43.2 |
| 16 | GGGSG | TVPCPVPSTPPTPSPSTPPTPSPSC | 14 | 118.8 |
| | | CHP | | |
| 17 | GGGSG | TVPCPVPSTP | 15 | 54 |
| 18 | GGGSG | TPSPSTPPTP | 16 | 54 |
| 19 | GGGSG | TPSPSCCHP | 17 | 50.4 |
| 20 | GGGSG | VPCPVPPPPPCCHPR | 18 | 72 |
| 21 | GGGSG | VPCPVPP | 19 | 43.2 |
| 22 | GGGSG | PPPCCHP | 20 | 43.2 |
| 23 | GGGSG | PCPVPPPPPCCHP | 21 | 64.8 |
| 24 | GGGSG | RWPESPKAQASSVPTAQPQAEG | 22 | 295.2 |
| | | SLAKATTAPATTRNTGRGGEEK | | |
| | | KKEKEKEEQEERETKTPECPSHT | | |
| | | QPLGVYLLTP | | |
| 25 | GGGSG | KTPECPSHTQPLGVYLLTP | 23 | 86.4 |
| 26 | GGGSG | PECPSHTQPLGVYLLTP | 24 | 79.2 |
| 27 | GGGSG | RWPESPKAQASSVP | 25 | 68.4 |
| 28 | GGGSG | SVCSRDFTPP | 26 | 54 |
| 29 | GGGSG | PLPVIAELPPKVSVFVPPRDGFFG | 27 | 111.6 |
| | | NP | | |
| 30 | GGGSG | PLPVIAELPP | 28 | 54 |
| 31 | GGGSG | ELPPKVSVFVPP | 29 | 61.2 |
| 41BB_ | GGGSG | LQDPCSNCPAGTFCDNNRNQICS | 30 | 197.6 |
| 1 | | PCPPNSFSSAGGQRTCDICRQCK | | |
| | | GVFRTRKECSSTSNAECDCTPGF | | |
| | | HCLGAGCSMCEQDCKQGQELT | | |
| | | KKGCKDCCFGTFNDQKRGICRP | | |
| | | WTNCSLDGKSVLVNGTKERDV | | |
| | | VCGPSPADLSPGASSVTPPAPAR | | |
| | | EPGHSP | | |
| 41BB_ | GGGSG | DCKQGQELTKKGCKDCCFGTFN | 31 | 138.3 |
| 2 | | DQKRGICRPWTNCSLDGKSVLV | | |
| | | NGTKERDVVCGPSPADLSPGASS | | |
| | | VTPPAPAREPGHSP | | |
| 41BB_ | GGGSG | GPSPADLSPGASSVTPPAPAREP | 32 | 106.7 |
| 3 | | GHSP | | |
| CD8a | GGGSG | ASSGTTTPAPRPPTPAPTIASQPL | 33 | 194.4 |
| | | SLRPEACRPAAGGAVHTRGLDF | | |
| | | ACD | | |
| CD27_ | GGGSG | TPAPKSCPERHYWAQGKLCCQM | 34 | 324.4 |
| 1 | | CEPGTFLVKDCDQHRKAAQCDP | | |
| | | CIPGVSFSPDHHTRPHCESCRHC | | |
| | | NSGLLVRNCTITANAECACRNG | | |
| | | WQCRDKECTECDPLPNPSLTARS | | |
| | | SQALSPHPQPTHLPYVSEMLEAR | | |
| | | TAGHMQTLADFRQLPARTLSTH | | |
| | | WPPQRSLCSSDFIR | | |
| CD27_ | GGGSG | PLPNPSLTARSSQALSPHPQPTHL | 35 | 270 |
| 2 | | PYVSEMLEARTAGHMQTLADFR | | |
| | | QLPARTLSTHWPPQRSLCSSDFIR | | |
| CD28 | GGGSG | IEVMYPPPYLDNEKSNGTIIHVK | 36 | 158.4 |
| | | GKHLCPSPLFPGPSKP | | |
| short_ | GGGSG | ESKYGPPSPPSP | 37 | 61.2 |
| nod- | | | | |
| isulf | | | | |
| Dap10 | GGGSG | QTTPGERSSLPAFYPGTSGSCSG | 38 | 65.8 |
| | | CGSLSLP | | |
| GS_ | GGGSG | ESKYGPPSPPSP | 39 | 61.2 |
| short | | | | |
| nod- | | | | |
| isulf | | | | |
| ICOS | GGGSG | DPPPFKVTLTGGYLHIYESQLCC | 40 | 111.6 |
| | | QLK | | |
| OX40 | GGGSG | EDRDPPATQPQETQGPPARPITV | 41 | 190.8 |
| | | QPTEAWPRTSQGPSTRPVEVPGG | | |
| | | RA | | |
| Inter- | GGGSG | ESKYGPPCPPCPGQPREPQVYTL | 42 | 104.5 |
| medi- | | PPSQEEMTKNQVSLTCLVKGFYP | | |
| ate | | SDIAVEWESNGQPENNYKTTPPV | | |
| | | LDSDGSFFLYSRLTVDKSRWQE | | |
| | | GNVFSCSVMHEALHNHYTQKSL | | |
| | | SLSLGK | | |
| Long | GGGSG | ESKYGPPCPPCPAPPVAGPSVFLF | 43 | 138.9 |
| (CH2CH3) | | PPKPKDTLMISRTPEVTCVVVDV | | |
| | | SQEDPEVQFNWYVDGVEVHNA | | |
| | | KTKPREEQFQSTYRVVSVLTVLH | | |
| | | QDWLNGKEYKCKVSNKGLPSSI | | |
| | | EKTISKAKGQPREPQVYTLPPSQ | | |
| | | EEMTKNQVSLTCLVKGFYPSDIA | | |
| | | VEWESNGQPENNYKTTPPVLDS | | |
| | | DGSFFLYSRLTVDKSRWQEGNV | | |
| | | FSCSVMHEALHNHYTQKSLSLSL | | |
| | | GK | | |
| Short | GGGSG | ESKYGPPCPPCP | 44 | 43.2 |
| (IgG4 | | | | |
| Hinge) | | | | |
| 48 | GGGSG | KCCVECPPCP | 74 | 54 |
| 49 | GGGSG | DKTHTCPPCP | 75 | 54 |
| 50 | GGGSG | ERKCCVECPPCP | 78 | 61.2 |
| 51 | GGGSG | ERKCCVECPPCPAP | 79 | 68.4 |
| 52 | GGGSG | PPPCPRCPEPKSCDTPPPCPRCPAP | 82 | 108 |

TABLE 2
Exemplary HA-target epitope spacers

| Target/ | | | Target/ | |
|---|---|---|---|---|
| epitope | | | epitope | |
| Spacer | Linker | | Spacer | Å |
| No. or | (SEQ ID | | SEQ | (with |
| Name | NO: 77) | Sequence | ID NO: | linker) |
| 32 | GGGSG | PRCPAP | 58 | 39.6 |
| 33 | GGGSG | PPCPRCPAP | 59 | 50.4 |
| 34 | GGGSG | DTPPPCPRCPAP | 60 | 61.2 |
| 35 | GGGSG | KSCDTPPPCPRCPAP | 61 | 72 |
| 36 | GGGSG | PEPKSCDTPPPCPRC | 62 | 82.8 |
| | | PAP | | |
| 37 | GGGSG | PRCPEPKSCDTPPPC | 63 | 93.6 |
| | | PRCPAP | | |
| 38 | GGGSG | PPCPRCPEPKSCDTP | 64 | 104.4 |
| | | PPCPRCPAP | | |
| 39 | GGGSG | DTPPPCPRCPEPKSC | 65 | 115.2 |
| | | DTPPPCPRCPAP | | |
| 40 | GGGSG | KSCDTPPPCPRCPEP | 66 | 126 |
| | | KSCDTPPPCPRCPAP | | |
| 41 | GGGSG | PEPKSCDTPPPCPRC | 67 | 136.8 |
| | | PEPKSCDTPPPCPRC | | |
| | | PAP | | |
| 42 | GGGSG | PRCPEPKSCDTPPPC | 68 | 147.6 |
| | | PRCPEPKSCDTPPPC | | |
| | | PRCPAP | | |
| 43 | GGGSG | PPCPRCPEPKSCDTP | 69 | 158.4 |
| | | PPCPRCPEPKSCDTP | | |
| | | PPCPRCPAP | | |
| 44 | GGGSG | DTPPPCPRCPEPKSC | 70 | 169.2 |
| | | DTPPPCPRCPEPKSC | | |
| | | DTPPPCPRCPAP | | |
| 45 | GGGSG | KSCDTPPPCPRCPEP | 71 | 180 |
| | | KSCDTPPPCPRCPEP | | |
| | | KSCDTPPPCPRCPAP | | |
| 46 | GGGSG | PEPKSCDTPPPCPRC | 72 | 190.8 |
| | | PEPKSCDTPPPCPRC | | |
| | | PEPKSCDTPPPCPRC | | |
| | | PA P | | |
| 47 | GGGSG | PRCPEPKSCDTPPPC | 73 | 201.6 |
| | | PRCPEPKSCDTPPPC | | |
| | | PRCPEPKSCDTPPPC | | |
| | | PRCPAP | | |

### Methods of Spacer Selection

The disclosure provides a method for designing a spacer sequence for each CAR-epitope binding pair to achieve a cell-to-cell distance of between about 17 nm and about 25 nm.

In some embodiments of the methods for designing a spacer sequence of the disclosure, the method comprises: (a) expressing a CAR that specifically binds to a target in a first cell; wherein the CAR comprises a test spacer sequence; (b) contacting the first cell to a second cell, wherein the second cell expresses the target; (c) measuring the cell-to-cell distance between the first cell and the second cell; (d) selecting the test spacer when the cell-to-cell distance is between about 17 nm and about 25 nm. In some embodiments, a set of test spacers comprises the test spacer.

In some embodiments of the methods for designing a spacer sequence of the disclosure, the cell expressing the CAR is an immune cell. In some embodiments, the immune cell is a natural killer (NK) cell, a lymphocyte, a B cell and/or a T cell. In some embodiments, the immune cell is a precursor cell. In some embodiments, the immune cell is a hematopoietic stem cell (HSC). In some embodiments, the immune cell is a differentiated cell, optionally, differentiated from an induced pluripotent stem cell (iPSC).

In some embodiments of the methods for designing a spacer sequence of the disclosure, the cell expressing the target is an antigen presenting cell (APC). In some embodiments, the (APC) is a monocyte, a dendritic cell, a macrophage, a B cell or a target cell of the disclosure. In some embodiments, the APC is modified to express the target. In some embodiments, the APC is not modified to express the target. In some embodiments, the target comprises a naturally-occurring antigen. In some embodiments, the target comprises a cancer antigen. In some embodiments, the target comprises a synthetic antigen or a neoantigen. In some embodiments, the target comprises a target or epitope spacer sequence. In some embodiments, the target or epitope spacer sequence mimics an extension of the target from the cell expressing it during an in vitro assay, in accordance with the presentation of the target or epitope, for example, as part of an MHC. Exemplary target or epitope spacers are provided in Table 2 of the disclosure.

In some embodiments of the methods for designing a spacer sequence of the disclosure, the cell expressing the target is a primary cell. In some embodiments, the primary cell is isolated or derived from a biological sample. In some embodiments, the biological sample is obtained from a subject. In some embodiments, the primary cell is modified. In some embodiments, the primary cell is unmodified.

In some embodiments of the methods for designing a spacer sequence of the disclosure, the cell expressing the target is a cultured cell. In some embodiments, the cultured cell is isolated or derived from a biological sample. In some embodiments, the biological sample is obtained from a subject. In some embodiments, the cultured cell is modified. In some embodiments, the cultured cell is immortalized. In some embodiments, the cultured cell is modified to express the target. In some embodiments, the expression of the target by the cultured cell is modified. In some embodiments, the expression of the target by the cultured cell is increased, decreased, or rendered inducible.

In some embodiments of the methods for designing a spacer sequence of the disclosure, the method comprises: (a) expressing a first CAR that specifically binds to a target in a first cell, wherein the CAR comprises a first test spacer sequence; (b) expressing a second CAR that specifically binds to the target in a second cell, wherein the CAR comprises a second test spacer sequence; (c) contacting the first cell and the second cell to a third cell, wherein the third cell expresses the target; (d) measuring the cell-to-cell distance between the first cell and the third cell and measuring the cell-to-cell distance between the second cell and the third cell; and (d) selecting the first test when the cell-to-cell distance is between about 17 nm and about 25 nm or selecting the second test when the cell-to-cell distance is between about 17 nm and about 25 nm. In some embodiments, a set of test spacers comprises the first test spacer and the second test spacer. In some embodiments, the set of spacers comprises at least one spacer comprising a sequence of SEQ ID NO: 1-44. In some embodiments, the set of spacers comprises at least 2, 3, 4, 5, 6, 7, 8, 9, 10, 15, 20, 25, 30, 35, 40 or 44 spacers comprising a sequence of SEQ ID NO: 1-44. In some embodiments, the set of spacers comprises each one of the sequences of SEQ ID NO: 1-44.

In some embodiments of the methods for designing a spacer sequence of the disclosure, the method comprises selecting the test spacer when the cell-to-cell distance is between about 17 nm to about 22 nm. In some embodiments, the method comprises selecting the first test when the cell-to-cell distance is between about 17 nm to about 22 nm.

In some embodiments of the methods for designing a spacer sequence of the disclosure, the method comprises selecting the test spacer when the cell-to-cell distance is about 20 nm. In some embodiments, the method comprises selecting the first test when the cell-to-cell distance is between about 20 nm.

In some embodiments of the methods for designing a spacer sequence of the disclosure, the first test spacer and the second test spacer comprise one or more of distinct sequences, distinct lengths, distinct secondary geometry, distinct tertiary geometry, distinct flexibility, distinct charge profile, distinct hydrophobicity, distinct hydrophilicity, and distinct immunogenicity.

In some embodiments of the methods for designing a spacer sequence of the disclosure, the first test spacer and the second test spacer comprise distinct lengths.

In some embodiments of the methods for designing a spacer sequence of the disclosure, the test spacer comprises a length of between about 2 nm to about 20 nm. In some embodiments, the test spacer comprises a length of between about 3 nm to about 19 nm. In some embodiments, the test spacer comprises a length of between about 4 nm to about 17 nm.

In some embodiments of the methods for designing a spacer sequence of the disclosure, the first test spacer or the second test spacer comprise a sequence of SEQ ID NO: 1-44. In some embodiments of the methods for designing a spacer sequence of the disclosure, the first test spacer or the second test spacer comprise a sequence at least 90%, 91%, 92%, 930%, 94%, 95%, 96%, 97%, 98%, or 99% identical to the sequence of SEQ ID NO: 1-44.

In some embodiments of the methods for designing a spacer sequence of the disclosure, the method comprises (a) expressing a target on a surface of a target cell, wherein the target comprises a linear epitope and wherein the linear epitope is displayed at varying distances from the surface of the target cell, (b) contacting the target cell with a CAR-expressing cell, wherein the CAR selectively binds the target and wherein the CAR comprises a test spacer sequence and (c) measuring the cell-to-cell distance between the first cell and the second cell; (d) selecting the test spacer sequence when the cell-to-cell distance is between about 17 nm and about 25 nm.

In some embodiments of the methods for designing a spacer sequence of the disclosure, the method comprises varying a length of a spacer sequence within a CAR and a spacer sequence within a target to conduct pairwise analysis of T cell function, including, but not limited to, measuring target cell lysis and measuring cytokine production.

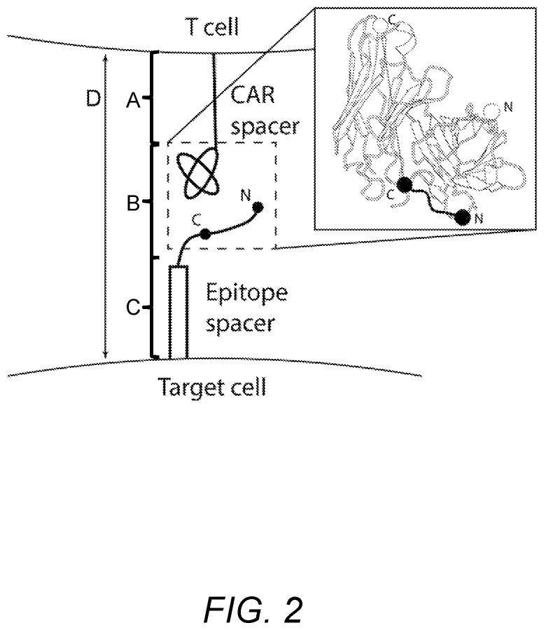

FIG. 2 is a diagram showing cell-to-cell distance between an exemplary HA epitope (target) on the surface of a target cell and a binding element of a CAR expressed on the surface of a CAR T cell. In this example, the cell-to-cell distance D is the sum of A+B+C, where: Distance A is the contribution to the cell-to-cell distance of the CAR spacer (including a GS linker), Distance B is the contribution to the cell-to-cell distance of the epitope binding element binding with the epitope, Distance C is the contribution to the cell-to-cell distance of the epitope spacer, which also includes a GS linker.

Spacer design methods of the disclosure may include experimentally estimating or determining the cell-to-cell distance during a binding interaction, and optionally, may estimate, determine, validate or confirm empirical data by modeling or computer modeling. Spacer design methods of the disclosure may include various empirically measured distances, such as distances determined by X-ray crystallography studies.

Spacer sequences of the disclosure may be used to generate one or more libraries of spacer sequences. A spacer library of the disclosure may include spacer sequences suitable for use with a CAR that specifically binds a particular target, for example, a plurality of spacer sequences that when incorporated into the target-specific CAR, result in a cell-to-cell distance between the CAR-expressing cell and a target-expressing cell of between about 17 nm and about 25 nm. In some embodiments, a spacer library of the disclosure includes spacer sequences suitable for use with a CAR that specifically binds a particular target, for example, a plurality of spacer sequences that when incorporated into the target-specific CAR, result in a cell-to-cell distance between the CAR-expressing cell and a target-expressing cell of between about 17 nm and about 22 nm. In some embodiments, a spacer library of the disclosure includes spacer sequences suitable for use with a CAR that specifically binds a particular target, for example, a plurality of spacer sequences that when incorporated into the target-specific CAR, result in a cell-to-cell distance between the CAR-expressing cell and a target-expressing cell of about 20 nm.

### Cells

The disclosure provides a cell comprising one or more of a CAR of the disclosure, a sequence encoding a CAR of the disclosure, a vector encoding a CAR of the disclosure, and a composition of the disclosure (including a pharmaceutical composition of the disclosure).

In some embodiments of the disclosure, a cell of the compositions and methods of the disclosure is a human cell. In some embodiments, a cell of the compositions and methods of the disclosure is a non-human cell. In some embodiments, a cell of the compositions and methods of the disclosure is a mammalian cell, a non-human primate cell, a rodent cell, a rat cell, a mouse cell, or a hybridoma thereof.

In some embodiments of the disclosure, a cell of the compositions and methods of the disclosure is an immune cell. In some embodiments, the immune cell is a natural killer (NK) cell, a lymphocyte, a B cell and/or a T cell. In some embodiments, the immune cell is a precursor cell. In some embodiments, the immune cell is a hematopoietic stem cell (HSC). In some embodiments, the immune cell is a differentiated cell, optionally, differentiated from an induced pluripotent stem cell (iPSC).

In some embodiments of the disclosure, a cell of the compositions and methods of the disclosure is an antigen presenting cell (APC). In some embodiments, an APC of the disclosure includes, but is not limited to, a monocyte, a dendritic cell, a macrophage, a B cell or a target cell of the disclosure. In some embodiments, the APC is modified to express the target. In some embodiments, the APC is not modified to express the target. In some embodiments, the target comprises a naturally-occurring antigen. In some embodiments, the target comprises a cancer antigen. In some embodiments, the target comprises a synthetic antigen or a neoantigen. In some embodiments, the target comprises a target or epitope spacer sequence. In some embodiments, the target or epitope spacer sequence mimics an extension of the target from the cell expressing it during an in vitro assay, in accordance with the presentation of the target or epitope, for example, as part of an MHC. Exemplary target or epitope spacers are provided in Table 2 of the disclosure.

In some embodiments of the disclosure, a cell of the compositions and methods of the disclosure is a primary cell. In some embodiments, the primary cell is isolated or derived from a biological sample. In some embodiments, the biological sample is obtained from a subject. In some embodiments, the primary cell is modified. In some embodiments, the primary cell is unmodified.

In some embodiments of the disclosure, a cell of the compositions and methods of the disclosure is a cultured cell. In some embodiments, the cultured cell is isolated or derived from a biological sample. In some embodiments, the biological sample is obtained from a subject. In some embodiments, the cultured cell is modified. In some embodiments, the cultured cell is immortalized. In some embodiments, the cultured cell is modified to express the target. In some embodiments, the expression of the target by cultured cell is modified. In some embodiments, the expression of the target by cultured cell is increased, decreased, or rendered inducible.

In some embodiments of the disclosure, a cell of the compositions and methods of the disclosure is a disease cell. In some embodiments, the disease cell is isolated or derived from a biological sample. In some embodiments, the biological sample is obtained from a subject. In some embodiments, the biological sample is a biopsy. In some embodiments, the disease cell is isolated or derived from a tumor. In some embodiments the tumor is a malignant tumor. In some embodiments, the disease cell is a cancerous, transformed or malignant cell.

In some embodiments of the disclosure, a cell of the compositions and methods of the disclosure is a modified cell. In some embodiments, the modified cell expresses a CAR of the disclosure. In some embodiments, the modified cell is a human cell. In some embodiments, the modified cell is a human T cell or a human NK cell. In some embodiments, the modified human T cell or modified human NK cell comprises a CAR comprising a spacer sequence of SEQ ID NO: 1-44.

The disclosure provides amino acid and nucleic acid sequences encoding a chimeric antigen receptor (CAR). In some embodiments, a dataset comprises a library of amino acid or nucleic acid sequences for use in empirical evaluation of spacer selection models. In some embodiments, a dataset comprises a library of amino acid or nucleic acid sequences encoding one or more spacers for use in empirical evaluation of spacer selection models. Sequences of the dataset may be physically selected from the library for assembly into amino acid or nucleic acid sequences encoding a spacer of the disclosure. Sequences of the dataset may be physically selected from the library for assembly into amino acid or nucleic acid sequences encoding a CAR of the disclosure. Sequences of the dataset may be physically selected from the library for assembly into amino acid or nucleic acid sequences encoding a target of the disclosure, particularly, for use in a spacer selection method of the disclosure. Sequences may be physically selected using a variety of techniques, e.g., manually, microfluidically, or robotically selected.

### Targets

In some embodiments of the CARs of the disclosure, the CAR selectively and specifically binds to a target. In some embodiments, the target is expressed on a target cell of the disclosure. In some embodiments, the target comprises an antigen. In some embodiments, the target comprises an epitope. In some embodiments, the epitope is linear. In some embodiments, the epitope is non-linear. In some embodiments, the epitope is conformational or discontinuous.

In some embodiments of the CARs of the disclosure, the CAR selectively and specifically binds to a target. In some embodiments, the target comprises one or more of a protein, a carbohydrate, or a glycolipid molecule.

In some embodiments of the CARs of the disclosure, the CAR selectively and specifically binds to a target. In some embodiments, the target comprises an antigen. In some embodiments the target comprises a tumor antigen or a marker of a tumor microenvironment (TME). In some embodiments, the target comprises a cell-type marker or a biomarker.

In some embodiments of the CARs of the disclosure, the CAR selectively and specifically binds to a target expressed on a cancer cell. In some embodiments, the cancer cell is isolated or derived from one or more of the following cancers: Acute Lymphoblastic Leukemia (ALL), Acute Myeloid Leukemia (AML), Adolescent Cancer, Adrenocortical Carcinoma AIDS-Related Cancers (including, but not limited to, Kaposi Sarcoma (Soft Tissue Sarcoma), AIDS-Related Lymphoma (Lymphoma), and Primary CNS Lymphoma (Lymphoma)), Anal Cancer, Appendix Cancer (also Gastrointestinal Carcinoid Tumor), Astrocytoma, Atypical Teratoid/Rhabdoid Tumor, Central Nervous System Cancer, Basal Cell Carcinoma of the Skin (also Skin Cancer), Bile Duct Cancer, Bladder Cancer, Bone Cancer (includes Ewing Sarcoma and Osteosarcoma and Malignant Fibrous Histiocytoma), Brain Tumors (also Brain Cancer), Breast Cancer, Bronchial Tumors (Lung Cancer), Burkitt Lymphoma-(also Non-Hodgkin Lymphoma), Carcinoid Tumor (also Gastrointestinal tumor), Carcinoma of Unknown Primary, Cardiac (Heart) Tumors, Central Nervous System Cancer Atypical Teratoid/Rhabdoid Tumor (Brain Cancer), Medulloblastoma and Other CNS Embryonal Tumors (Brain Cancer), Germ Cell Tumor Brain Cancer), Primary CNS Lymphoma, Cervical Cancer, Childhood Cancers, Rare Cancers of Childhood, Cholangiocarcinoma (also Bile Duct Cancer), Chordoma (Bone Cancer), Chronic Lymphocytic Leukemia (CLL), Chronic Myelogenous Leukemia (CML), Chronic Myeloproliferative Neoplasms, Colorectal Cancer, Craniopharyngioma (Brain Cancer), Cutaneous T-Cell Lymphoma (also Lymphoma and Mycosis Fungoides and Sézary Syndrome), Ductal Carcinoma In Situ (DCIS) (also Breast Cancer), Embryonal Tumors, Medulloblastoma and Other Central Nervous System (Brain Cancer), Endometrial Cancer (Uterine Cancer), Ependymoma (Brain Cancer), Esophageal Cancer, Esthesioneuroblastoma (Head and Neck Cancer), Ewing Sarcoma (Bone Cancer), Extracranial Germ Cell Tumor, Extragonadal Germ Cell Tumor, Eye Cancer, Intraocular Melanoma, Retinoblastoma, Fallopian Tube Cancer, Gallbladder Cancer, Gastric (Stomach) Cancer, Gastrointestinal Carcinoid Tumor, Gastrointestinal Stromal Tumors (GIST) (Soft Tissue Sarcoma), Germ Cell Tumors, Childhood Central Nervous System Germ Cell Tumors (Brain Cancer), Childhood Extracranial Germ Cell Tumors, Extragonadal Germ Cell Tumors, Ovarian Germ Cell Tumors, Testicular Cancer, Gestational Trophoblastic Disease, Hairy Cell Leukemia, Head and Neck Cancer, Heart Tumors, Hepatocellular (Liver) Cancer, Histiocytosis (Langerhans Cell), Hodgkin Lymphoma, Hypopharyngeal Cancer (Head and Neck Cancer), Intraocular Melanoma, Islet Cell Tumors, Pancreatic Neuroendocrine Tumors, Kaposi Sarcoma (Soft Tissue Sarcoma), Kidney (Renal Cell) Cancer, Langerhans Cell Histiocytosis, Laryngeal Cancer (Head and Neck Cancer), Leukemia, Lip and Oral Cavity Cancer (Head and Neck Cancer), Liver Cancer, Lung Cancer (Non-Small Cell, Small Cell, Pleuropulmonary Blastoma, and Tracheobronchial Tumor), Lymphoma, Male Breast Cancer, Melanoma, Intraocular (Eye) Melanoma, Merkel Cell Carcinoma (Skin Cancer), Mesothelioma (Malignant), Metastatic Cancer, Metastatic Squamous Neck Cancer with Occult Primary (Head and Neck Cancer), Midline Tract Carcinoma With NUT Gene Changes, Mouth Cancer (Head and Neck Cancer), Multiple Endocrine Neoplasia Syndromes, Multiple Myeloma/Plasma Cell Neoplasms, Mycosis Fungoides (Lymphoma), Myelodysplastic Syndromes, Myelodysplastic/Myeloproliferative Neoplasms, Myelogenous Leukemia, Chronic (CML), Myeloid Leukemia, Acute (AML), Myeloproliferative Neoplasms, Chronic, Nasal Cavity and Paranasal Sinus Cancer (Head and Neck Cancer), Nasopharyngeal Cancer (Head and Neck Cancer), Neuroblastoma, Non-Hodgkin Lymphoma, Non-Small Cell Lung Cancer, Oral Cancer, Lip and Oral Cavity Cancer and Oropharyngeal Cancer (Head and Neck Cancer), Osteosarcoma and Undifferentiated Pleomorphic Sarcoma of Bone Treatment, Ovarian Cancer, Pancreatic Cancer, Pancreatic Neuroendocrine Tumors (Islet Cell Tumors), Papillomatosis (Childhood Laryngeal), Paraganglioma, Paranasal Sinus and Nasal Cavity Cancer (Head and Neck Cancer), Parathyroid Cancer, Penile Cancer, Pharyngeal Cancer (Head and Neck Cancer), Pheochromocytoma, Pituitary Tumor, Plasma Cell Neoplasm/Multiple Myeloma, Pleuropulmonary Blastoma (Lung Cancer), Pregnancy and Breast Cancer, Primary Central Nervous System (CNS) Lymphoma, Primary Peritoneal Cancer, Prostate Cancer, Rare Cancers of Childhood, Rectal Cancer, Recurrent Cancer, Renal Cell (Kidney) Cancer, Retinoblastoma, Rhabdomyosarcoma, Childhood (Soft Tissue Sarcoma), Salivary Gland Cancer (Head and Neck Cancer), Sarcoma, Childhood Rhabdomyosarcoma (Soft Tissue Sarcoma), Childhood Vascular Tumors (Soft Tissue Sarcoma), Ewing Sarcoma (Bone Cancer), Kaposi Sarcoma (Soft Tissue Sarcoma), Osteosarcoma (Bone Cancer), Soft Tissue Sarcoma, Uterine Sarcoma, Sézary Syndrome (Lymphoma), Skin Cancer, Small Cell Lung Cancer, Small Intestine Cancer, Soft Tissue Sarcoma, Squamous Cell Carcinoma of the Skin (Skin Cancer), Squamous Neck Cancer with Occult Primary and Metastatic (Head and Neck Cancer), Stomach (Gastric) Cancer, T-Cell Lymphoma (also Cutaneous Lymphoma, Mycosis Fungoides and Sezary Syndrome), Testicular Cancer, Throat Cancer (Head and Neck Cancer), Nasopharyngeal Cancer, Oropharyngeal Cancer, Hypopharyngeal Cancer, Thymoma and Thymic Carcinoma, Thyroid Cancer, Tracheobronchial Tumors (Lung Cancer), Transitional Cell Cancer of the Renal Pelvis and Ureter (Kidney (Renal Cell) Cancer), Unknown Primary Carcinoma, Ureter and Renal Pelvis, Transitional Cell Cancer (Kidney (Renal Cell) Cancer, Urethral Cancer, Uterine Cancer (also Endometrial), Uterine Sarcoma, Vaginal Cancer, Vascular Tumors (Soft Tissue Sarcoma), Vulvar Cancer, Wilms Tumor and Other Childhood Kidney Tumors, and Young Adult Cancer.

### Binding Elements

In some embodiments of the CARs of the disclosure, the CAR comprises an extracellular element. In some embodiments of the CARs of the disclosure, the extracellular element comprises a binding element. In some embodiments of the CARs of the disclosure, the extracellular element comprises at least one binding element. In some embodiments of the CARs of the disclosure, the extracellular element comprises one or more binding elements. In some embodiments of the CARs of the disclosure, the extracellular element comprises a bi-specific binding element. In some embodiments of the CARs of the disclosure, the extracellular element comprises a tri-specific binding element. In some embodiments of the CARs of the disclosure, the extracellular element comprises a multi-specific binding element.

Exemplary binding elements of the disclosure may comprise any form. Exemplary binding elements of the disclosure selectively and specifically bind to a target of the disclosure. In some embodiments, the binding element comprises one or more modifications to increase binding affinity, selectivity or specificity when compared to an unmodified binding element.

Exemplary binding elements of the disclosure may comprise any form. Exemplary binding elements of the disclosure selectively and specifically bind to a target of the disclosure. In some embodiments, the binding element comprises a protein scaffold. In some embodiments, the protein scaffold comprises one or more sequences isolated or derived from a human fibronectin protein. In some embodiments, the protein scaffold comprises one or more modifications to increase binding affinity, selectivity or specificity when compared to an unmodified protein scaffold. In some embodiments, the binding element or protein scaffold comprises a monobody.

Exemplary binding elements of the disclosure may comprise any form. Exemplary binding elements of the disclosure selectively and specifically bind to a target of the disclosure. In some embodiments, the binding element comprises an antibody or a functional fragment thereof. In some embodiments, the antibody comprises a monoclonal antibody. In some embodiments, the antibody comprises a single domain or domain antibody. In some embodiments, the antibody comprises a camelid antibody. In some embodiments, an antibody fragment comprises a molecule other than an intact antibody that includes a portion of an intact antibody that binds the antigen to which the intact antibody binds. Examples of antibody fragments include, but are not limited to, Fab, Fab′, F(ab′)₂, and Fv fragments, linear antibodies, single domain antibodies such as sdAb (either V_{L} or V_{H}), camelid VHH domains, and multi-specific antibodies formed from antibody fragments.

Exemplary binding elements of the disclosure may comprise any form. Exemplary binding elements of the disclosure selectively and specifically bind to a target of the disclosure. In some embodiments, the binding element comprises an antibody mimetic or a functional fragment thereof.

Exemplary binding elements of the disclosure may comprise any form. Exemplary binding elements of the disclosure selectively and specifically bind to a target of the disclosure. In some embodiments, the binding element comprises a single-chain variable fragment (scFv). In some embodiments of the disclosure, a scFv comprises a variable region of a heavy chain (VH) and a variable region of a light chain (VL), linked by a flexible peptide linker.

Exemplary binding elements of the disclosure may comprise any form. Exemplary binding elements of the disclosure selectively and specifically bind to a target of the disclosure. In some embodiments, the binding element comprises a fusion protein.

A binding element of the disclosure specifically and/or selectively binds to a target of the disclosure. A multispecific binding element ofthe disclosure specifically and/or selectively binds to two or more targets of the disclosure.

In some embodiments of the disclosure, a target comprises an epitope. In some embodiments, the target comprises a linear epitope, a continuous epitope, a discontinuous epitope, and/or a conformational epitope.

In some embodiments of the disclosure, a target comprises an antigen.

In some embodiments of the disclosure, a target comprises an amino acid sequence. In some embodiments, the target does not comprise an amino acid.

### Method of Making: Polynucleotides

The disclosure provides methods of producing polynucleotides of the disclosure, such as DNA vectors of the disclosure and their subcomponents, as well as packaging vectors and plasmids of the disclosure. Standard molecular biology techniques may be used to assemble the polynucleotides of the disclosure. Polynucleotides can be chemically synthesized.

### Method of Making: Packaged Viral Capsids

The disclosure includes methods of making viral capsids containing polynucleotides of the disclosure. In general, viral capsids of the disclosure may be produced by supplying cells with packaging polynucleotides of the disclosure. The packaging polynucleotides may be supplied to packaging cells as plasmids. The packaging cells may be cultured to produce the viral capsids containing polynucleotides of the disclosure. Preferably the packaged viral capsids are replication incompetent.

A variety of commercially available kits are suitable for producing packaged viral capsids of the disclosure. Examples include MISSION® Lentiviral Packaging Mix (available from Millipore Sigma); LV-Max Lentiviral Packaging Mix (available from ThermoFisher Scientific).

Viral capsid produced by packaging cells may be purified for use in downstream methods, such as delivery to cells for use in production of polypeptides, delivery to cells for use in cell-based therapies, or delivery to subjects for gene therapy methods. Purification may include processing to eliminate contaminants from host cells or culture media. Purification steps may include steps based on physical and/or chemical characteristics of the plasmids. Chemical characteristics may include, for example, hydrophilicity-hydrophobicity. Physical characteristics may include, for example, size. Examples of purification strategies based on particle size include density-gradient ultracentrifugation, ultrafiltration, precipitation, two-phase extraction systems and size exclusion chromatography. In some cases, precipitation may be employed together with centrifugation, e.g., using polyethylene glycol, ammonium sulfate or calcium phosphate. In some cases, aqueous two-phase separation systems with PEG, dextran or polyvinyl alcohol may be used. In some cases, membrane-based tangential flow filtration techniques are used; examples include ultrafiltration, diafiltration and microfiltration. In other embodiments, chromatographic means may be used for purifying viral capsids. In still other embodiments, immunoaffinity methods may be used to capture capsids using monoclonal antibodies having specificity to the relevant capsids. See Morenweiser, R., “Downstream processing of viral vectors and vaccines,” Gene Therapy (2005) 12, S103-S110 (2005), the entire disclosure of which is incorporated herein by reference.

Examples of suitable viral capsids include, but are not limited to, adenovirus, retrovirus, Lentivirus, Sendai virus vector, a baculovirus, Epstein Barr virus, a papovavirus, a vaccinia virus, a herpes simplex virus, and an adeno-associated virus (AAV).

### Method of Making: Cells

The disclosure provides methods of making a modified cell to express a CAR of the disclosure.

In certain embodiments, the disclosure provides a method of making a therapeutic cell for use in treating a subject in need of a cell therapy. In one aspect, the disclosure provides a method of generating or preparing a therapeutic cell that expresses a CAR.

In certain embodiments, the polynucleotides of the disclosure are maintained as extrachromosomal polynucleotides in the host cell. In certain embodiments, the polynucleotides of the disclosure are present in a vector (e.g., expression vector) in the host cell. In certain embodiments, the polynucleotides of the disclosure or a subset or subcomponents thereof, are integrated into a chromosome of the host cell.

Various methods can be used to introduce the expression vector encoding polynucleotides of the disclosure into cells to produce cells of the disclosure. See for example, Green, et al., Molecular cloning: A laboratory manual. Cold Spring Harbor, NY: Cold Spring Harbor Laboratory Press (2014).

Methods of modifying polynucleotides known in the art may be used to make or modify host cells with polynucleotides of the disclosure. Examples include targeted homologous recombination (e.g. “Hit and run”, “double-replacement”), site specific recombinases (e.g. the Cre recombinase and the Flp recombinase), PB transposases (e.g. Sleeping Beauty, piggyBac, To 12 or Frog Prince), genome editing by engineered nucleases (e.g. meganucleases, Zinc finger nucleases (ZFNs), transcription-activator like effector nucleases (TALENs) and CRISPR/Cas system) and genome editing using recombinant adeno-associated virus (rAAV) platform. Agents for introducing nucleic acid alterations to a gene of interest can be designed using publicly available sources or obtained commercially from Transposagen, Addgene and Sangamo Biosciences. Vectors of the disclosure may make use of these methods for integrating polynucleotides of the disclosure into a host genome. Polynucleotides and vectors of the disclosure may include polynucleotides encoding polypeptides required for implementation of these methods for integrating polynucleotides of the disclosure into a host genome.

Various approaches suitable for integrating a polynucleotide(s) into a host cell genome are known in the art, including random integration or site-specific integration (e.g., a “landing pad” approach); see, e.g., Zhao, M. et al. (2018) Appl. Microbiol. Biotechnol. 102:6105-6117; Lee, J. S. et al. (2015) Sci. Rep. 5:8572; and Gaidukov, L. et al. (2018) Nucleic Acids Res. 46:4072-4086. Vectors of the disclosure may make use of these methods for integrating polynucleotides of the disclosure into a host genome. Vectors of the disclosure may include polynucleotides encoding polypeptides required for implementation of these methods for integrating polynucleotides of the disclosure into a host genome.

Host cells may be cultured using methods and compositions known in the art. Examples of commercially available media suitable for culturing host cells of the disclosure include Ham's F10 (Sigma), Minimal Essential Medium ((MEM), (Sigma), RP MI-1640 (Sigma), and Dulbecco's Modified Eagle's Medium ((DMEM), Sigma).

Culture media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. Culture conditions, such as temperature, pH, and the like, will be apparent to the ordinarily skilled artisan.

### Pharmaceutical Compositions

The disclosure provides a pharmaceutical composition comprising a CAR of the disclosure, a sequence comprising or encoding a CAR of the disclosure, a vector comprising a sequence encoding a CAR of the disclosure or a cell comprising one or more of the proceeding, in combination with a pharmaceutically acceptable carrier.

In some embodiments of the pharmaceutical compositions of the disclosure, a pharmaceutically acceptable carrier comprises a sterile saline or salt solution that balances or mimics physiological conditions of a subject's blood stream or the local conditions of a tumor microenvironment.

In some embodiments of the pharmaceutical compositions of the disclosure, a pharmaceutically acceptable carrier comprises an adjuvant for modulation of the immune system or immune response of the subject.

In some embodiments of the pharmaceutical compositions of the disclosure, a pharmaceutically acceptable carrier comprises an agent to stabilize or prevent degradation of amino acid or nucleic acid sequences in the composition.

In some embodiments of the pharmaceutical compositions of the disclosure, a pharmaceutically acceptable carrier comprises an agent to optimize solubility, stability, tonicity, or viscosity of the composition. In some embodiments, a pharmaceutically acceptable carrier comprises one or more of a bulking agent, a surfactant, and a chelating agent.

In some embodiments of the pharmaceutical compositions of the disclosure, a pharmaceutically acceptable carrier comprises an agent to adjust the pH of the pharmaceutical composition prior to administration to the subject. In some embodiments, a pharmaceutically acceptable carrier comprises a buffering agent

In some embodiments of the pharmaceutical compositions of the disclosure, a pharmaceutically acceptable carrier comprises an agent to maintain or increase the viability or an activity of a cell or any component of a cell of the composition. In some embodiments, a pharmaceutically acceptable carrier comprises an immunostimulatory cytokine, including, but not limited to IL-2, IL-7, IL-12 and/or IL-15. In some embodiments, a pharmaceutically acceptable carrier comprises a chemokine.

In some embodiments of the pharmaceutical compositions of the disclosure, a pharmaceutically acceptable carrier comprises one or more of a liposome, a viral capsid, a micelle, a polymersome, or a nanoparticle. In some embodiments of the pharmaceutical compositions of the disclosure, a pharmaceutically acceptable carrier comprises an allogeneic cell. In some embodiments of the pharmaceutical compositions of the disclosure, a pharmaceutically acceptable carrier comprises an allogeneic T-cell.

In some embodiments of the pharmaceutical compositions of the disclosure, a pharmaceutically acceptable carrier comprises one or more pharmaceutically acceptable salts. Pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluensulfonic, methanesulfonic, ethane dislfonic, oxalic, isethionic, and the like. For example, CARs of the disclosure may be modulated by small molecules which may be provided as pharmaceutically acceptable salts.

### Therapeutic Methods

The disclosure provides a use of a CAR of the disclosure, a nucleic acid of the disclosure, a vector of the disclosure, a cell of the disclosure, or a pharmaceutical composition of the disclosure for manufacture of a medicament.

The disclosure provides a use of a CAR of the disclosure, a nucleic acid of the disclosure, a vector of the disclosure, a cell of the disclosure, or a pharmaceutical composition of the disclosure for treatment or prevention of a disease or disorder. In some embodiments, the disease or disorder comprises a cancer. In some embodiments, the cancer occurs in circulating blood or lymph fluid (a liquid tumor). In some embodiments, the cancer occurs in a sheet of cells or in a non-fluid organ (a solid tumor). In some embodiments, the cancer is a metastatic cancer. In some embodiments, the cancer comprises a cancer described in the disclosure.

The disclosure provides a method of treating a disease or disorder, comprising administering to a subject a therapeutically-effective amount of a CAR of the disclosure, a nucleic acid of the disclosure, a vector of the disclosure, a cell of the disclosure, or a pharmaceutical composition of the disclosure, wherein a severity of a sign or symptom of the disease or disorder is decreased, thereby treating the disease or disorder. In some embodiments, the disease or disorder comprises a cancer. In some embodiments, the cancer occurs in circulating blood or lymph fluid (a liquid tumor). In some embodiments, the cancer occurs in a sheet of cells or in a non-fluid organ (a solid tumor). In some embodiments, the cancer is a metastatic cancer. In some embodiments, the cancer comprises a cancer described in the disclosure.

In some embodiments of the disclosure, a sign of a disease or disorder is an objective indication of the disease or disorder. A sign as well as a change in a severity of a sign may be measured by objective means, including, but not limited to, physical examination, body scans (MRI, cat scan, ct-scan, X-ray), biopsy (liquid or solid), genetic testing, metabolite analysis, blood analysis, urine analysis, and the like. Signs may be interpreted by medical professionals.

In some embodiments of the disclosure, a symptom of a disease or disorder is a subjective experience of the subject indication of the disease or disorder. A symptom as well as a change in a severity of a symptom may be measured by subjective means, including, but not limited to, comparative states from before disease onset to present or from before treatment to after treatment. Exemplary states that may be examined include, but are not limited to: pain level, energy (or fatigue), mobility, nausea, appetite, and specific affiliations related to the cancer type (e.g., local swelling or organ function).

The disclosure provides a method of preventing a disease or disorder, comprising administering to a subject a therapeutically-effective amount of a CAR of the disclosure, a nucleic acid of the disclosure, a vector of the disclosure, a cell of the disclosure, or a pharmaceutical composition of the disclosure, wherein an onset or a relapse of a sign or symptom of the disease or disorder is delayed or inhibited, thereby preventing the disease or disorder. In some embodiments, the disease or disorder comprises a cancer. In some embodiments, the cancer occurs in circulating blood or lymph fluid (a liquid tumor). In some embodiments, the cancer occurs in a sheet of cells or in a non-fluid organ (a solid tumor). In some embodiments, the cancer is a metastatic cancer. In some embodiments, the cancer comprises a cancer described in the disclosure.

In some embodiments of the disclosure, an onset or a relapse of a sign or a symptom of a disease or disorder is delayed when the onset or relapse occurs after a predicted time period (e.g., a prognosis based on known or average timing across similar populations). In some embodiments of the disclosure, an onset or a relapse of a sign or a symptom of a cancer is delayed when the subject's anticipated cancer free survival duration was 3 years and the subject has been cancer-free for more than three years. In some embodiments of the disclosure, an onset or a relapse of a sign or a symptom of a disease or disorder is inhibited when signs of the disease or disorder under consideration (which may be specific to the disease or condition) are no longer detectable. In some embodiments of the disclosure, an onset or a relapse of a sign or a symptom of a liquid tumor is inhibited when cancer cells are not detectable in the subject's circulating blood or lymph fluid.

The disclosure provides methods of treating a subject in need of a cell therapy. The methods may include administering to the subject a therapeutically effective amount of a composition comprising a CAR T cell of the disclosure.

In some embodiments, a method includes the steps of: (i) administering to the subject a therapeutically effective amount of a pharmaceutical composition comprising a therapeutic cell encoding a therapeutic polypeptide product of interest (e.g., a CAR) and a pharmaceutically acceptable carrier; (ii) monitoring the effectiveness of the treatment; and (iii) repeating (i) and (ii) as needed. In some embodiments, the CAR may be administered together with a pharmaceutically acceptable carrier.

In some embodiments, the disclosure provides a method for treating a cancer, e.g., a tumor, in a subject in need thereof. The methods may include administering to the subject having a cancer a therapeutically effective amount of a pharmaceutical composition comprising a therapeutic cell encoding a CAR of the disclosure. The CAR may be administered together with a pharmaceutically acceptable carrier.

Exemplary cancers that may be treated using a pharmaceutical composition disclosed herein include, but are not limited to, melanomas, lymphomas, sarcomas, and cancers of the colon, kidney, stomach, bladder, brain (e.g., gliomas, glioblastomas, astrocytomas, medulloblastomas), prostate, bladder, rectum, esophagus, pancreas, liver, lung, breast, uterus, cervix, ovary, blood (e.g., acute myeloid leukemia, acute lymphoid leukemia, chronic myeloid leukemia, chronic lymphocytic leukemia, Burkitt's lymphoma, EBV-induced B-cell lymphoma).

In some embodiments, autologous cells are administered to a subject.

In some embodiments, allogenic cells are administered to a subject.

A subject of the disclosure may be of any age. In some embodiments, a subject of the methods of the disclosure is an adult. In some embodiments, an adult is aged 18-80 years. In some embodiments, a subject of the methods of the disclosure is a senior. In some embodiments, a senior is aged at least 80 years. In some embodiments, a subject ofthe methods of the disclosure is an adolescent. In some embodiments, an adolescent is aged 12-18 years. In some embodiments, a subject of the methods of the disclosure is a child. In some embodiments, a child is aged 5-11 years. In some embodiments, a subject of the methods of the disclosure is a young child. In some embodiments, a young child is aged 2-5 years. In some embodiments, a subject of the methods of the disclosure is a baby. In some embodiments, a baby is aged less than 2 years.

A subject of the disclosure may have one or two X chromosomes. A subject of the disclosure may have a Y chromosome.

A subject of the disclosure may have been diagnosed with the disease or disorder prior to initiation of the methods of the disclosure. In some embodiments, a subject of the disclosure is at risk of developing a disease or disorder of the disclosure. In some embodiments, a subject is identified as being “at risk” based on one or more of genetic testing, family history, work or home exposures, and level of health, which, cumulatively, provide an assessment of risk.

A subject of the disclosure may have been provided a first medical intervention to treat or prevent the disease or disorder, which may have been partially effective or ineffective. In some embodiments, the subject relapsed following administration of a first medical intervention, and, therefore, the subject is in need of treatment according to the methods of the disclosure.

A subject of the disclosure may have been identified as not responsive to existing treatments prior to initiation of a method of the disclosure. In some embodiments, a subject of the disclosure may be determined to be non-responsive to standard treatments based on one or more of genetic or biomarker testing, family history, level of health, and past experience with a particular intervention, which, cumulatively, provide an assessment of responsiveness to treatment.

A subject of the disclosure may receive a second medical intervention simultaneously or sequentially with the compositions and methods of the disclosure.

In some embodiments, a subject of the disclosure is human. In some embodiments, a subject of the disclosure is a non-human primate. In some embodiments, a subject of the disclosure is a mammal. In some embodiments, a subject of the disclosure is a horse, cow, sheep, dog, cat, pig, chicken, guinea pig, rodent, rat, or mouse.

### Nucleotides

“Nucleic acid,” and in particular a DNA or RNA molecule, refers only to the primary and secondary structure of the molecule, and does not limit it to any particular tertiary forms. Thus, this term includes double-stranded DNA found, inter alia, in linear or circular DNA molecules (e.g., restriction fragments), plasmids, supercoiled DNA and chromosomes. In discussing the structure of particular double-stranded DNA molecules, sequences are provided according to the normal convention of writing the sequence left to right in the 5′ to 3′ direction along the non-transcribed strand of DNA (i.e., the strand having a sequence homologous to the messenger RNA or mRNA). Unless otherwise indicated, all nucleic acid and nucleotide sequences are written left to right in 5′ to 3′ orientation.

Nucleotides are referred to by their commonly known one-letter symbols recommended by the IUPAC-IUB Biochemical Nomenclature Commission. Accordingly, ‘A’ represents adenine, ‘C’ represents cytosine, ‘G’ represents guanine, ‘T’ represents thymine, and ‘U’ represents uracil.

“Polynucleotide” means polymers of nucleotides of any length or type, including ribonucleotides, deoxyribonucleotides, analogs thereof, or mixtures thereof. This term refers to the primary structure of the molecule. The term includes double- and single-stranded nucleic acids, including deoxyribonucleic acid (“DNA”) and ribonucleic acid (“RNA”). It also includes modified, for example by alkylation and/or by capping, and unmodified forms of the polynucleotide.

In some embodiments, a polynucleotide includes a DNA, e.g., a DNA inserted in a vector. In some embodiments, a polynucleotide includes an mRNA. In some embodiments, the mRNA is a synthetic mRNA. In some embodiments, the synthetic mRNA includes at least one unnatural nucleobase. In some embodiments, all nucleobases of a certain class have been replaced with unnatural nucleobases (e.g., all uridines in a polynucleotide can be replaced with an unnatural nucleobase, e.g., 5-methoxy uridine).

“Expression vector” means a plasmid, virus, or other nucleic acid designed for polypeptide expression in a cell. The vector or construct is used to introduce a gene into a host cell whereby the vector will interact with polymerases in the cell to express the protein encoded in the vector/construct. The expression vector may exist in the cell extra chromosomally or may be integrated into the chromosome. Expression vectors may include additional sequences which render the vector suitable for replication and integration in prokaryotes, eukaryotes, or preferably both (e.g., shuttle vectors). The polynucleotides of the disclosure may be provided as components of expression vectors.

“Cloning vector” means a plasmid, virus, or other nucleic acid designed for producing copies of a polynucleotide. Cloning vectors may contain transcription and translation initiation sequences, transcription and translation termination sequences and a polyadenylation signal. Such constructs may typically include a 5′ LTR, a tRNA binding site, a packaging signal, an origin of second-strand DNA synthesis, and a 3′ LTR or a portion thereof. The polynucleotides of the disclosure may be provided as components of cloning vectors, which may be used to produce the polynucleotides of the disclosure.

“Promoter” refers to a nucleotide sequence which indicates where transcription of a gene is initiated and in which direction transcription will continue.

“Encoding” or the like refers to the inherent property of specific sequences of nucleotides in a polynucleotide (e.g., a gene, cDNA, or mRNA) to serve as templates for synthesis of other polymers and macromolecules in biological processes having either a defined sequence of nucleotides (e.g., rRNA, tRNA and mRNA) or a defined sequence of amino acids. Thus, a gene, cDNA, or RNA, encodes a protein if transcription and translation of mRNA corresponding to that gene produces the protein in a cell or other biological system. Both the coding strand and the non-coding strand can be referred to as encoding the protein or other product of that gene or cDNA.

Unless otherwise specified, a nucleotide sequence “encoding an amino acid sequence,” e.g., a polynucleotide “encoding” a chimeric polypeptide, defined below of the present disclosure, includes all nucleotide sequences that are degenerate versions of each other and that encode the same amino acid sequence.

### Polypeptides

Amino acids are referred to by either their commonly known three letter symbols or by the one-letter symbols recommended by the IUPAC-IUB Biochemical Nomenclature Commission. The amino acid residues are abbreviated as follows, where the abbreviations are shown in parentheses: alanine (Ala; A), asparagine (Asn; N), aspartic acid (Asp; D), arginine (Arg; R), cysteine (Cys; C), glutamic acid (Glu; E), glutamine (Gln; Q), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

Amino acid sequences are written left to right in amino to carboxy orientation.

“Polypeptide” is used in its broadest sense to refer to a sequence of amino acid subunits. In some embodiments, a “peptide” can be less than or equal to 50 amino acids long, e.g., about 5, 10, 15, 20, 25, 30, 35, 40, 45, or 50 amino acids long. “Polypeptide,” refers to proteins, polypeptides, and peptides of any length, size, structure, or function. “Polypeptide,” “peptide,” and “protein” are used interchangeably to refer to polymers of amino acids of any length.

The polypeptide may include naturally or synthetically created or modified amino acids, for example, disulfide bond formation, glycosylation, lipidation, acetylation, phosphorylation, or any other manipulation or modification, such as conjugation with a labeling component. Also included within the definition are, for example, polypeptides in which one or more amino acid residues are artificial chemical analogs of a corresponding naturally occurring amino acid (including, for example, synthetic amino acids such as homocysteine, ornithine, p-acetylphenylalanine, D-amino acids, and creatine), as well as other modifications known in the art. Polypeptides also include gene products, homologs, orthologs, paralogs, fragments and other equivalents, variants, and analogs of the foregoing. A polypeptide can be a single polypeptide or can be a multi-molecular complex such as a dimer, trimer or tetramer. Polypeptides also include single-chain or multi-chain polypeptides. Disulfide linkages may be present in multi-chain polypeptides. Polypeptides described can be chemically synthesized or recombinantly expressed.

The polypeptides of the disclosure may include additional residues or moieties at the N-terminus, C-terminus, internal to the polypeptide, or a combination thereof, these additional residues or moieties are not included in determining the percent identity of the polypeptides of the disclosure relative to the reference polypeptide. Such residues may be any residues suitable for an intended use, including but not limited to tags.

“Tags” may, for example, include detectable moieties (e.g., fluorescent proteins, antibody epitope tags, etc.), therapeutic agents, purification tags (His tags, etc.), linkers, ligands suitable for purposes of purification, ligands to drive localization of the polypeptide, and/or peptide domains that add functionality to the polypeptides.

“Chimeric polypeptide” refers to any polypeptide including a first amino acid sequence derived from a first source, bonded, covalently or non-covalently, to a second amino acid sequence derived from a second source, where the first and second source are not the same. A first source and a second source that are not the same may include two different biological entities, or two different proteins from the same biological entity, or a biological entity and a non-biological entity. A chimeric protein may include, for example, a protein derived from at least 2 different biological sources. A biological source may include any non-synthetically produced nucleic acid or amino acid sequence (e.g., a genomic or cDNA sequence, a plasmid or viral vector, a native virion or a mutant or analog of any of the above). A synthetic source may include a protein or nucleic acid sequence produced chemically and not by a biological system (e.g., solid phase synthesis of amino acid sequences). A chimeric protein may also include a protein derived from at least 2 different synthetic sources or a protein derived from at least one biological source and at least one synthetic source. A chimeric protein may also include a first amino acid sequence derived from a first source, covalently or non-covalently linked to a nucleic acid, derived from any source or a small organic or inorganic molecule derived from any source. The chimeric protein may include a linker molecule between the first and second amino acid sequence or between the first amino acid sequence and the nucleic acid, or between the first amino acid sequence and the small organic or inorganic molecule.

“Fragment” of a polypeptide, or a “truncated polypeptide” refers to an amino acid sequence of a polypeptide that is shorter than the naturally-occurring sequence. In comparison to the naturally occurring polypeptide, the fragment can be N- and/or C-terminally deleted or can have any part of the polypeptide deleted. Thus, a fragment does not necessarily need to have only N- and/or C-terminal amino acids deleted. A polypeptide in which internal amino acids have been deleted with respect to the naturally occurring sequence is also considered a fragment. The polypeptide components of the disclosure may be provided as fragments or truncated polypeptides.

“Functional fragment” refers to a polypeptide fragment that retain s the function of the polypeptide. Accordingly, in some embodiments, a functional fragment of a bioactive peptide, e.g., an enzyme, retains the ability to catalyze a biological action, e.g., having a catalytic domain of the enzyme. Polypeptides of the disclosure may comprise or be provided as functional fragments.

“Functional variant” refers to a modified form of a polypeptide, fragment, or a member of a class of polypeptides, which maintains the function of the polypeptide. The polypeptide components of the disclosure may be provided as functional variants.

“Amino acid substitution” refers to replacing an amino acid residue present in a parent or reference sequence (e.g., a wild-type sequence) with another amino acid residue. An amino acid can be substituted, for example, via chemical peptide synthesis or through recombinant methods. For example, substituting an amino acid residue with an alternative amino acid residue may be conducted by substituting the codon encoding the first amino acid with a codon encoding the second amino acid. Polypeptides of the disclosure may comprise amino acid substitutions.

“Conservative amino acid substitution” is one in which one amino acid residue is replaced with an amino acid residue having a chemically similar side chain. Families of amino acid residues having similar side chains have been defined in the art, including acidic side chains (e.g., aspartic acid, glutamic acid), basic side chains (e.g., lysine, arginine, histidine), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, if an amino acid in a polypeptide is replaced with another amino acid from the same side chain family, the substitution is considered to be conservative. In another aspect, a string of amino acids can be conservatively replaced with a chemically similar string that differs in order and/or composition of side chain family members. The various polypeptide components of the disclosure may be provided with conservative amino acid substitutions.

“Non-conservative amino acid substitutions” include those in which (i) a residue having an electropositive side chain (e.g., Arg, His or Lys) is substituted for, or by, an electronegative residue (e.g., Glu or Asp), (ii) a hydrophilic residue (e.g., Ser or Thr) is substituted for, or by, a hydrophobic residue (e.g., Ala, Leu, Ile, Phe or Val), (iii) a cysteine or proline is substituted for, or by, any other residue, or (iv) a residue having a bulky hydrophobic or aromatic side chain (e.g., Val, His, Ile or Trp) is substituted for, or by, one having a smaller side chain (e.g., Ala or Ser) or no side chain (e.g., Gly). The various polypeptide components of the disclosure may be provided with non-conservative amino acid substitutions. The likelihood that one of the foregoing non-conservative substitutions can alter functional properties of the protein is also correlated to the position of the substitution with respect to functionally important regions of the protein: some non-conservative substitutions can accordingly have little or no effect on biological properties. The various polypeptide components of the disclosure may in some cases be provided with non-conservative amino acid substitutions that do not significantly alter the functionality of the altered components. The various polypeptide components of the disclosure may in some cases be provided with non-conservative amino acid substitutions that alter the functionality of the altered components, either by enhancing or reducing functionality.

### Sequence Identity or Similarity

“Conserved” refers to nucleotides of a polynucleotide sequence or amino acid residues of a polypeptide sequence that occur unaltered in the same position of two or more sequences being compared. Nucleotides or amino acids that are con served are those that are conserved amongst more related sequences than nucleotides or amino acids appearing elsewhere in the sequences. In some embodiments, two or more sequences are said to be “conserved” if they are at least about 30% identical, at least about 35% identical, at least about 40% identical, at least about 45% identical, at least about 50% identical, at least about 55%, at least about 60% identical, at least about 65% identical, at least about 70% identical, at least about 75% identical, at least about 80% identical, at least about 85% identical, at least about 90% identical, at least about 95% identical to one another, at least about 98% identical, or at least about 99% identical to one another. Conservation of sequence may apply to the entire length of a polynucleotide or polypeptide or may apply to a portion, region or feature thereof.

In some embodiments, two or more sequences are said to be “completely conserved” or “identical” if they are 100% identical to one another. In some embodiments, two or more sequences are said to be “highly conserved” if they are at least about 70% identical, at least about 75% identical, at least about 80% identical, at least about 85% identical, at least about 90% identical, at least about 95% identical to one another at least about 98% identical, or at least about 99% identical to one another.

“Homology” refers to the overall relatedness between polymeric molecules, e.g., between nucleic acid molecules and/or between polypeptide molecules. Homology encompasses both identity and similarity.

In some embodiments, polymeric molecules are considered to be “homologous” to one another if at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% of the monomers in the molecule are identical (exactly the same monomer) or are similar (conservative substitutions). “Homologous” necessarily refers to a comparison between at least two polynucleotide or polypeptide sequences. In various aspects, the disclosure includes polynucleotides and polypeptides that are homologous to the polynucleotides and polypeptides set forth herein.

“Identity” refers to the overall monomer conservation between polymeric molecules, e.g., between polypeptide molecules or polynucleotide molecules. “Identical” without any additional qualifiers, e.g., protein A is identical to protein B, implies the sequences are 100% identical (100% sequence identity). Describing two sequences as, e.g., “70% identical,” is equivalent to describing them as having, e.g., “70% sequence identity.”

When a position in a first sequence is occupied by the same amino acid as the corresponding position in a second sequence, then the molecules are identical at that position. The percent identity between the two sequences is a function of the number of identical positions shared by the sequences, accounting for the number of gaps, and the length of each gap, which needs to be introduced for optimal alignment of the two sequences. The comparison of sequences and determination of percent identity between two sequences can be accomplished using a mathematical algorithm.

In some embodiments, the percentage identity (% ID) of a first amino acid (or nucleic acid) sequence to a second amino acid (or nucleic acid) sequence is calculated as % ID=100 (Y/Z), where Y is the number of amino acid (or nucleobase) residues scored as identical matches in the alignment of the first and second sequences (as aligned by visual inspection or a particular sequence alignment program) and Z is the total number of residues in the second sequence. If the length of a first sequence is longer than the second sequence, the percent identity of the first sequence to the second sequence will be higher than the percent identity of the second sequence to the first sequence.

Calculation of the percent identity of two polypeptide sequences, for example, can be performed by aligning the two sequences for optimal comparison purposes. For example, gaps can be introduced in one or both of a first and a second polypeptide sequences for optimal alignment and non-identical sequences can be disregarded for comparison purposes. In some embodiments, the length of a sequence aligned for comparison purposes is at least 30%, at least 40%, at least 50%, at least 60%, at least 70%, at least 80%, at least 90%, at least 95%, or 100% of the length of the reference sequence. The amino acids at corresponding amino acid positions are then compared.

The generation of a sequence alignment for the calculation of a percent sequence identity is not limited to binary sequence-sequence comparisons exclusively driven by primary sequence data. Sequence alignments can be generated by integrating sequence data with data from heterogeneous sources such as structural data (e.g., crystallographic protein structures), functional data (e.g., location of mutations), or phylogenetic data. Software that integrates heterogeneous data to generate a multiple sequence alignment may be used; one example is T-Coffee, available at www.tcoffee.org, and alternatively available, e.g., from the European Bioinformatics Institute (EBI) at website ebi.ac.uk/Tools/psa. The final alignment used to calculate percent sequence identity can be curated either automatically or manually.

Software for alignment of polypeptide and nucleotide sequences is available. One program used to determine percent sequence identity is bl2seq, part of the BLAST suite of programs available from the U.S. government's National Center for Biotechnology Information BLAST web site (blast.ncbi.nlm.nih.gov). B12seq performs comparisons using either the BLASTN or BLASTP algorithm. BLASTN is used to compare nucleic acid sequences, while BLASTP is used to compare amino acid sequences. Other suitable programs are, e.g., Needle, Stretcher, Water, or Matcher, part of the EMBOSS suite of bioinformatics programs and also available from the EBI. Sequence alignments can be conducted using methods known in the art such as MAFFT, Clustal (ClustalW, Clustal X or Clustal Omega), MUSCLE, etc. Different regions within a single polynucleotide or polypeptide target sequence that aligns with a polynucleotide or polypeptide reference sequence can each have their own percent sequence identity. It is noted that the percent sequence identity value is rounded to the nearest tenth. For example, values from 80.11 to 80.14 are rounded down to 80.1, while values from 80.15 to 80.19 are rounded up to 80.2. It also is noted that the length value will always be an integer.

“Similarity” refers to the overall relatedness between polymeric molecules, e.g., between polynucleotide molecules and/or between polypeptide molecules. Calculation of percent similarity of polymeric molecules to one another can be performed in the same manner as a calculation of percent identity, except that calculation of percent similarity may account for conservative substitutions. Percentage of similarity is contingent on the comparison scale used; for example, whether the amino acids are compared, e.g., according to their evolutionary proximity, charge, volume, flexibility, polarity, hydrophobicity, aromaticity, isoelectric point, antigenicity, or combinations thereof.

### Cell Therapy

In some embodiments of the disclosure, a hematopoietic cell is a cell that arises from a hematopoietic stem cell. This includes, but is not limited to, myeloid progenitor cells, lymphoid progenitor cells, megakaryocytes, erythrocytes, mast cells, myeloblasts, basophils, neutrophils, eosinophils, macrophages, thrombocytes, monocytes, natural killer cells, T lymphocytes, B lymphocytes and plasma cells.

In some embodiments of the disclosure, a T-lymphocyte or T-cell means a hematopoietic cell that normally develops in the thymus. T-lymphocytes or T-cells of the disclosure include, but are not limited to, natural killer T cells, regulatory T cells, helper T cells, cytotoxic T cells, memory T cells, gamma delta T cells, and mucosal invariant T cells.

In some embodiments of the disclosure, an autologous cell is a cell obtained from the same individual to whom it may be administered as a therapy (the cell is autologous to the subject). Autologous cells of the disclosure include, but are not limited to, hematopoietic cells and stem cells, such as hematopoietic stem cells.

In some embodiments of the disclosure, an allogeneic cell is a cell obtained from an individual who is not the intended recipient of the cell as a therapy (the cell is allogeneic to the subject). Allogeneic cells of the disclosure may be selected from immunologically compatible donors with respect to the subject of the methods of the disclosure. Allogeneic cells of the disclosure may be modified to produce “universal” allogeneic cells, suitable for administration to any subject without unintended immunogenicity. Allogeneic cells of the disclosure include, but are not limited to, hematopoietic cells and stem cells, such as hematopoietic stem cells.

### General Terminology

“A,” “an” and “the” include their plural forms unless the context dictates otherwise.

“And” is used interchangeably with “or” unless expressly stated otherwise.

“And/or” is to be taken as specific disclosure of each of the two specified features or components with or without the other. Thus, “and/or” as used in a phrase such as “A and/or B,” includes “A and B,” “A or B,” “A” (alone), and “B” (alone). Likewise, “and/or,” as used in a phrase such as “A, B, and/or C,” is intended to encompass each of the following aspects: A, B, and C; A, B, or C; A or C; A or B; B or C; A and C; A and B; B and C; A (alone); B (alone); and C (alone).

“About” means approximately, roughly, around, or in the region of. When “about” is used with a numerical range, it modifies that range by extending the boundaries above and below the numerical values set forth. “About” can modify a numerical value above and below the stated value by a variance. As used throughout the disclosure, any numerical value that is disclosed in the context of “about” is also intended to be stated in the absence of the term “about”. For example, the term “about 17 nm” is intended to also disclose the term “17 nm”.

Numeric ranges are inclusive of the numbers defining the range. Where a range of values is stated, each intervening integer value, and each fraction thereof, between the recited upper and lower limits of that range is also specifically disclosed, as is each subrange between such values. The upper and lower limits of any range can independently be included in or excluded from the range, and each range where either, neither or both limits are included is also encompassed within the disclosure. Thus, ranges are understood to be shorthand for all of the values within the range, inclusive of the recited endpoints.

Where a value is explicitly stated, it is to be understood that equivalent values which are about the same quantity or amount as the stated value (or that lead to a substantially similar result by a substantially similar mechanism) are also within the scope of the disclosure.

Where a combination is disclosed, each subcombination of the elements of that combination is also specifically disclosed and is within the scope of the disclosure. Conversely, where different elements or groups of elements are individually disclosed, combinations thereof are also disclosed.

Where any element of a disclosure is disclosed as having a plurality of alternatives, examples of that disclosure in which each alternative is excluded singly or in any combination with the other alternatives are also disclosed; more than one element of a disclosure can have such exclusions, and all combinations of elements having such exclusions are hereby disclosed.

Unless the context clearly requires otherwise, throughout the description and the claims, the words ‘include,’ ‘including,’ and the like are meant to be construed in the sense of “including, but not limited to.”

Singular or plural words also include the plural and singular number, respectively.

“Above,” and “below” and words of similar import refer to this application as a whole and not to any particular portions of the application.

“Set” includes sets of one or more elements or objects. A“subset” of a set includes any number elements or objects from the set, from one up to all of the elements of the set.

Headings are included throughout the disclosure for reference and to aid in locating the various sections. These headings are not intended to limit the scope of the concepts described with respect to the headings. Such concepts may have applicability throughout the present specification.

Although the disclosure is described in some detail by way of illustration and example for purposes of clarity and understanding, certain changes and modifications may be practiced. Reference to “the disclosure” or the like is intended as a reference to any of a wide variety of embodiments of, or aspects of, the disclosure, and not as limiting the disclosure to a single embodiment or aspect.

The description and examples should not be construed as limiting the scope of the disclosure to the embodiments and examples described herein, but as encompassing all modifications and alternatives falling within the true scope and spirit of the disclosure.

### Devices

The disclosure provides a device comprising a CAR of the disclosure, a sequence comprising or encoding a CAR of the disclosure (including a spacer sequence), a vector comprising a sequence encoding a CAR of the disclosure (including a spacer sequence), a cell of the disclosure comprising any one or more of the foregoing, a composition of the disclosure comprising any one or more of the foregoing, and a pharmaceutical composition of the disclosure comprising any one or more of the foregoing.

Exemplary devices of the disclosure include, but are not limited to, a syringe, a needle, and a vial.

### Kits

The disclosure provides kits or articles of manufacture comprising one or more of CARs, sequences comprising or encoding CARs. A kit may, for example, include components for delivery of the polynucleotides to cells or to a subject. A kit may, for example, include components for delivery of cells, such as CAR T cells of the disclosure, to a subject. The polynucleotides may be provided in the kit as part of an expression vector configured for expression in a T cell. In certain embodiments, the kit or article of manufacture further comprises instructions for using the set of the polynucleotides to transform cells to express a gene of interest to produce a CAR if the disclosure. In certain embodiments, the kit or article of manufacture further comprises instructions for using the cells to treat a subject.

### EXAMPLES

### Example 1: Functional Benefit of Spacer Sequence Selection for CAR Efficacy

This study describes the effects of various Ig-derived or extracellular domain-derived spacers on inducing antigen-directed cytokine release and potency. An anti-HA scFv (clone 2E2) was used as the CAR binder and an HA peptide (YPYDVPDYA) was used as a model linear epitope that could be systematically displayed at different distances from the target cell membrane. This approach allowed the systematic evaluation of optimal cell-to-cell distance for CAR T cells.

The study used an in vitro screening strategy to evaluate the effects of CAR spacer length on inducing antigen-directed cytokine release and potency. The screening strategy included:
(i) varying the distance of an epitope from the surface of a target cell;
(ii) varying the distance of a corresponding epitope-binder (“binder”) from the surface of a CAR T cell;
(iii) evaluating pairwise combinations of a target cell and a CAR T cell for basic functional parameters (e.g., target-cell lysis and cytokine production).

Lentiviral constructs were generated with bi-cistronic expression constructs. The coding sequences for the following were linked in frame and placed under the control of an MND promoter:
(i) an HA-specific CAR,
(ii) a P2A self-cleaving peptide, and
(iii) EGFRt transduction marker (i.e., a truncated EGFR having only Domains III and IV and the transmembrane domain).

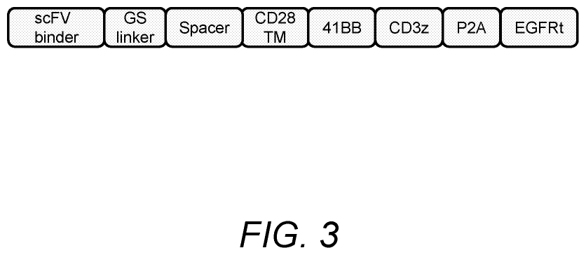

FIG. 3 illustrates the organization of the components of the constructs.

The HA-specific CARs used in the study included spacer sequences derived from human immunoglobulin (Ig) hinge regions or extracellular protein domains (ECD) coupled to a common Gly-Ser (GS) linker sequence.

To analyze the influence of spacer length on CAR T cell function, the length of the spacer was varied while other components in the HA-specific CARs were held constant. For example, the antigen-binding element was anti-HA scFv (clone 2E2); the transmembrane element was derived from the CD28 protein transmembrane domain; and intercellular element was derived from the 4-1BB protein costimulatory domain and the CD3 zeta signaling domain.

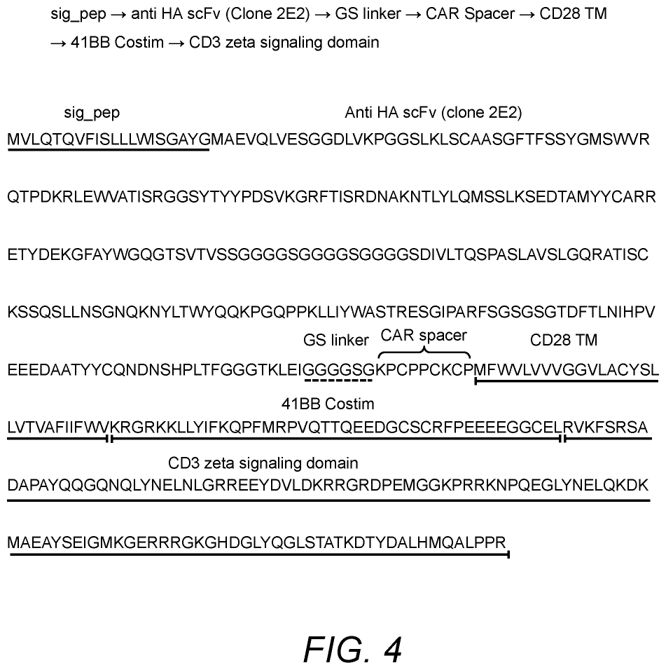

FIG. 4 is an example of an HA-specific CAR construct used in the study.

Sequences and lengths of the spacers used in the study are shown in Table 1. Spacer sequences are identified by a spacer number 1 through 31 or other shown designation. The total CAR spacer lengths are shown in angstroms, which includes both the linker sequence and spacer sequence.

Human primary T cells from two normal donors (Donor 1 and Donor 2) were obtained. HA-specific CARs were introduced into Donor 1 and Donor 2 T cells using the lentiviral constructs coding for the bi-cistronic expression cassettes.

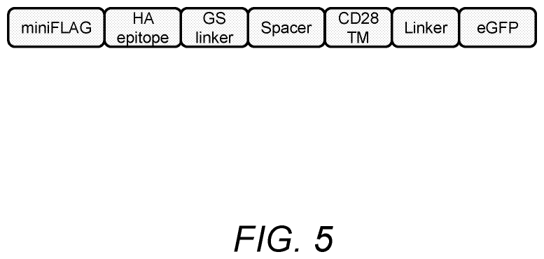

FIG. 5 provides an exemplary HA-epitope construct showing the organization of the different elements, and, in the context of this example, the construct that was used.

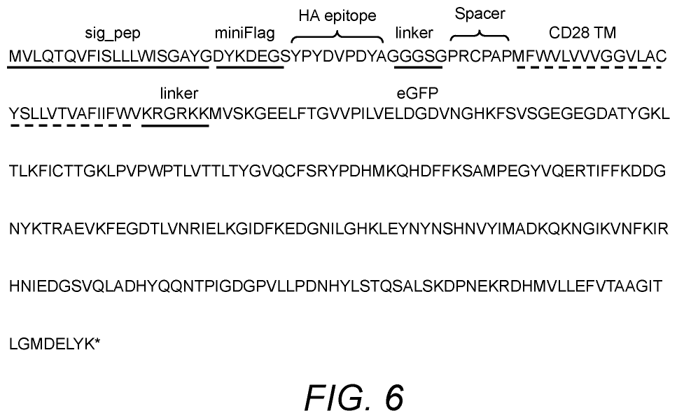

The HA-epitope expression constructs used in the study included spacer sequences derived human immunoglobulin (Ig) protein linked to a common GS linker sequence. To systematically vary the distance of the HA-epitope from the surface of the A549 target cell, the length of the spacer sequence was varied while other components in the expression constructs were held constant: miniFLAG tag sequence (for determining HA-epitope surface expression), HA epitope sequence, transmembrane domain derived from the CD28 protein, intracellular linker sequence, and the eGFP transduction marker. FIG. 6 is a schematic diagram of an example of an HA-epitope construct used in the study.

Sequences and lengths of the epitope spacers used in the study are shown in Table 2. Target epitopes are identified by a target epitope number 1 through 16, where 1 is assigned to the shortest epitope spacer length (i.e., target epitope will be closest to the membrane of the target cell) and 16 is assigned to the longest epitope length (i.e., the target epitope will be furthest from the target cell membrane). The total epitope spacer lengths are shown in angstroms, which includes both the linker sequence and spacer sequence.

A549-NLR cells were used as a target cell background for expression of HA-epitope constructs. Sixteen A549-NLR target cell lines were generated, each expressing the HA epitope at a different distance from the surface of the cell membrane.

In the functional assays described hereinbelow, the sixteen A549-NLR target cell lines are designated as target 1, target 2, target 3, etc. based on the target epitope number shown in Table 2. For example, target 1 expresses the most membrane proximal HA epitope, target 6 expresses the HA epitope at an intermediate membrane distance, and target 16 expresses the most membrane distal HA epitope.

The functional assays correlate CAR T cell mediated target cell killing and target-dependent cytokine secretion to assess the efficacy of pairwise combinations of HA epitope target cells and anti HA CAR T cells.

Area under the curve (AUC) values were calculated by integrating the area under normalized target cell killing curves. The lower the AUC value, the better the efficacy of the CAR T cell mediated killing.

Transduction efficiency of A549-NLR cells with the various length-controlled HA-epitope constructs was evaluated by percentage of transduction marker (GFP+), HA surface expression (% HATag+), and median mean fluorescence intensity (MFI).

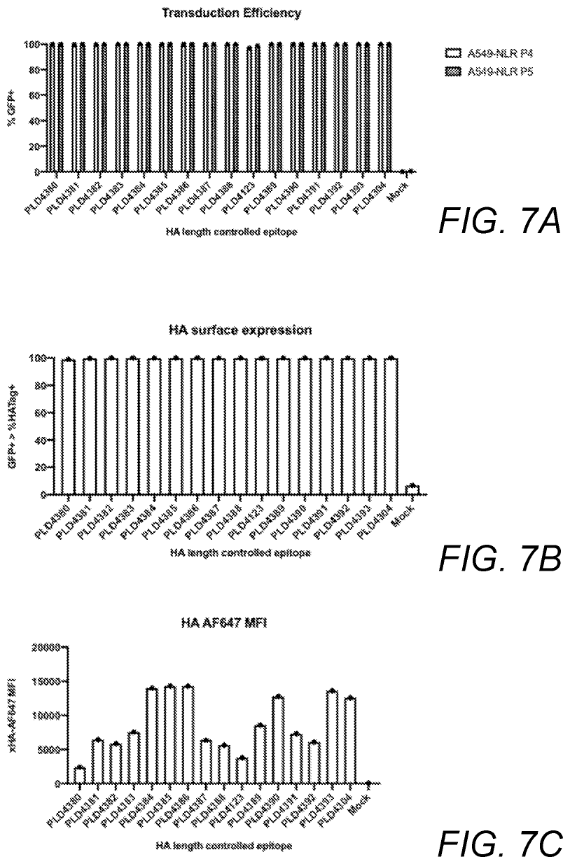

FIG. 7A is a plot showing the percentage of transduced target cells measured by expression of the transduction marker GFP on live A549-NLR cells. Transduced cells were monitored during expansion over passage 4 (P4) and passage 5 (P5). The data show that we were able to make 16 A549-NLR target cell lines each expressing a length-controlled HA epitope.

FIG. 7B is a plot showing the percentage of HA tag surface expression on transduced GFP+ cells. The data show that the expressed epitope is detectable on the A549-NLR target cells.

FIG. 7C is a plot showing the median fluorescence intensity (MFI) for bound anti-HA antibodies on live, transduced cells (GRP+ cells). The data show that there are some differences on a per cell line basis in the surface expression of the HA epitope.

Transduction efficiency of Donor 1 and Donor 2 cells with the various anti-HA CAR constructs was evaluated by percentage of transduction marker (EGFRt+) and median mean fluorescence intensity (MFI).

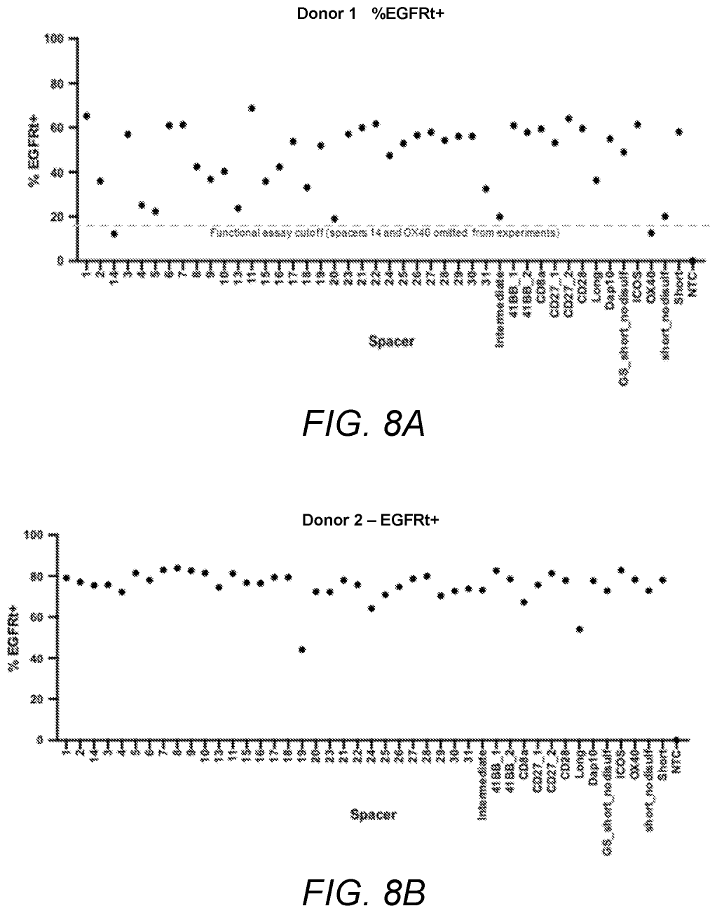

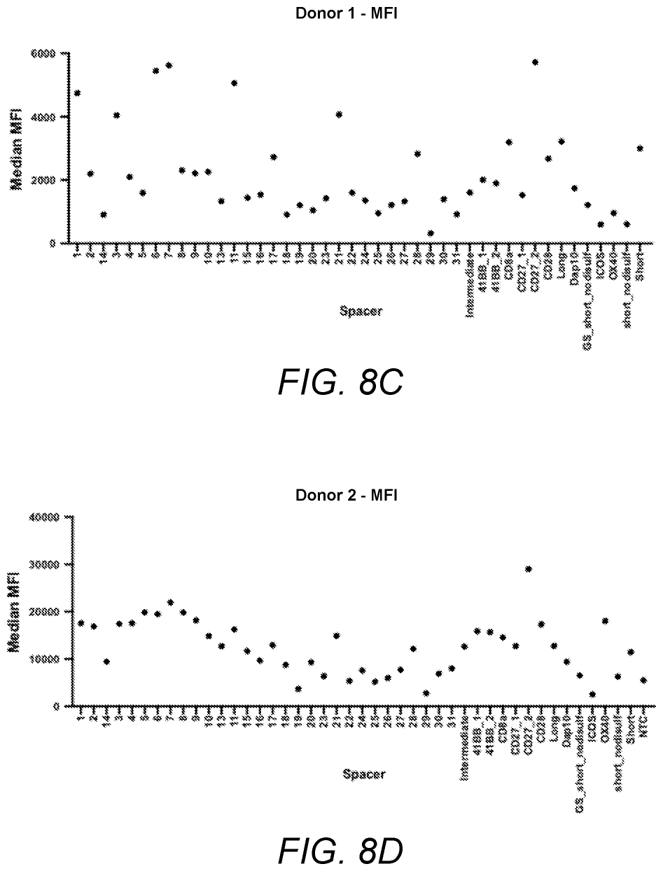

FIG. 8A and FIG. 8B are plots showing the percentage of transduced cells measured by surface expression of the transduction marker EGFRt on live primary T cells in Donor 1 and Donor 2, respectively. The anti-HA CARs used are listed on the x-axis (see Table 1). A functional assay cutoff was set below 20% and is indicated by the dashed line. Referring now to FIG. 8A, anti-HA CAR constructs that did not transduce well for Donor 1 were constructs with spacer 14 and spacer OX40. These two constructs were omitted from subsequent functional assay experiments. Referring now to FIG. 8B, all anti-HA CAR constructs transduced well for Donor 2 and were included in subsequent functional assay experiments.

FIG. 8C and FIG. 8D are plots showing the median fluorescence intensity (MFI) as a measure of transduction efficiency for bound HA-Fc protein on live, transduced cells (EGFRt+ cells) in Donor 1 and Donor 2, respectively.

A systematic screening strategy was used to establish the relationship between T cell efficacy and cell-to-cell distance between pairwise combinations of a CAR T cell and a target cell. Target cell killing and target-dependent cytokine production were used as indicators of CAR T cell efficacy.

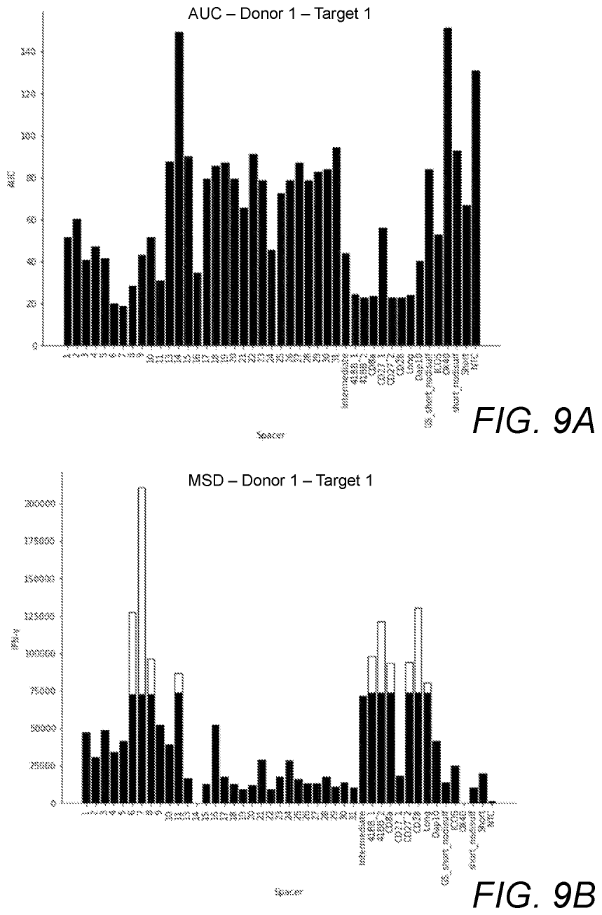

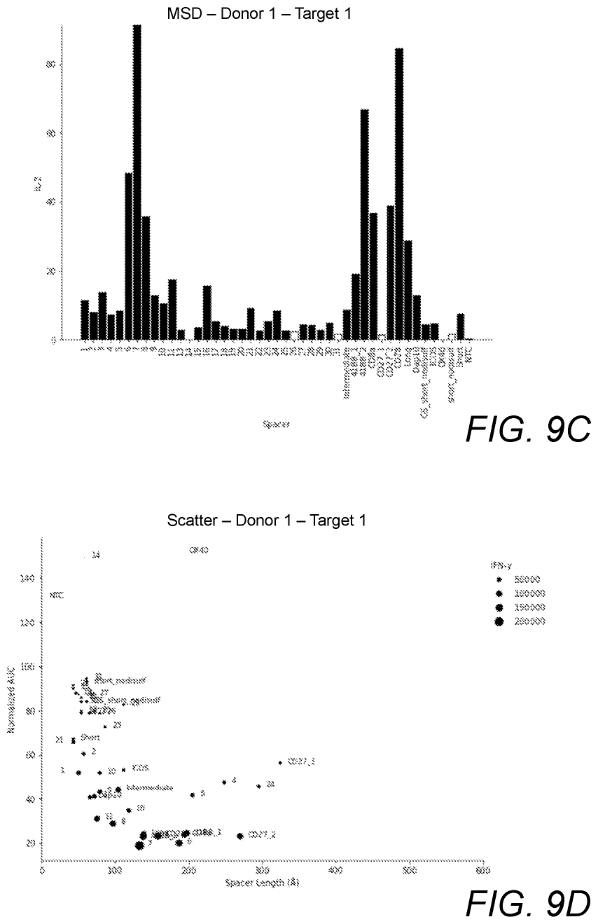

FIGS. 9A, 9B, 9C, and 9D illustrate the effects of indicated spacers on anti HA scFv-induced cytotoxicity in the presence of NucLight Red-labeled A549 target cells expressing the most membrane proximal HA target epitope (A549-HA target 1) in Donor 1.

Referring now to FIG. 9A is a plot showing the Area Under the Curve (AUC) calculated for IncuCyte killing curves of each CAR variant with the indicated spacer. Non-transduced primary T cells are indicated as “NTC.” The smaller the AUC value, the greater the anti-HA CAR T cell killing efficacy. In this example, Donor 1 CAR T cells expressing spacers 6 (spacer length 187.2 Å) and 7 (spacer length 133.2 Å) have the smallest AUC values, which indicates greater killing of A549-HA target 1, which expresses the most membrane proximal HA epitope.

Referring now to FIG. 9B and FIG. 9C are bar graphs showing concentrations of IFN-γ and IL-2, respectively, in co-culture media 24 hours post co-culturing. Cytokine secretion is directly correlated with target cell killing. For example, Donor 1 CAR T cells expressing spacers 6 and 7 which showed the best killing efficacy (see FIG. 9A) also show the highest levels of cytokine secretion.

Referring now to FIG. 9D is a scatter plot illustrating the effects of indicated spacer lengths on target-dependent cytokine secretion and killing AUC for the anti HA CAR-T cells when co-cultured with A549-HA target 1. Normalized AUC is on the y-axis and spacer length is on the x-axis. In the plot, dot size represents IFN-γ; the larger the dot size, the higher the level of IFN-7 secretion. The data shows, for example, that the optimal spacer length to kill the membrane proximal target 1 is about 130 Å, which correlates with the highest level of IFNγ secretion. The data shows a u-shaped curve, which indicates that for the given target as you move away from the optimal spacer length, either to the left or to the right on the x-axis, you are going to start losing killing efficacy and cytokine secretion (i.e., the data points start to go up for AUC and dot size for IFNγ decreases).

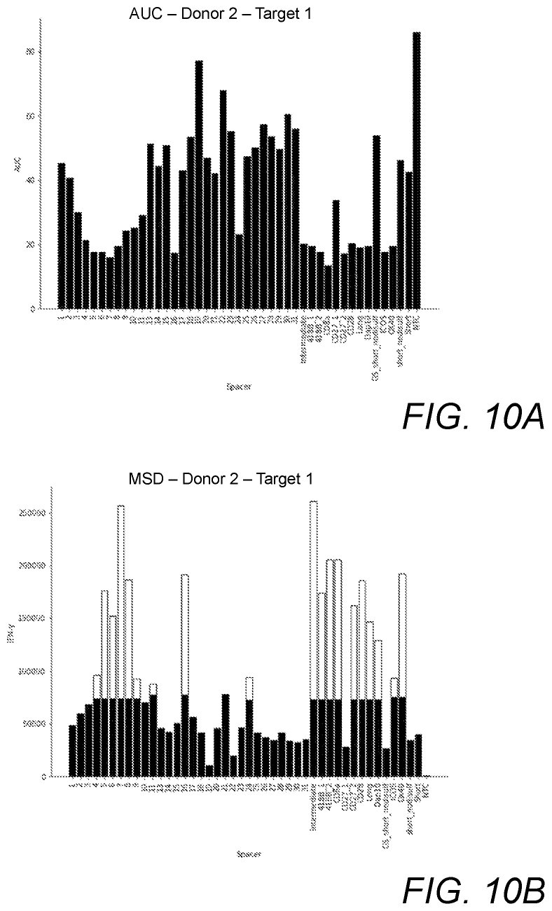

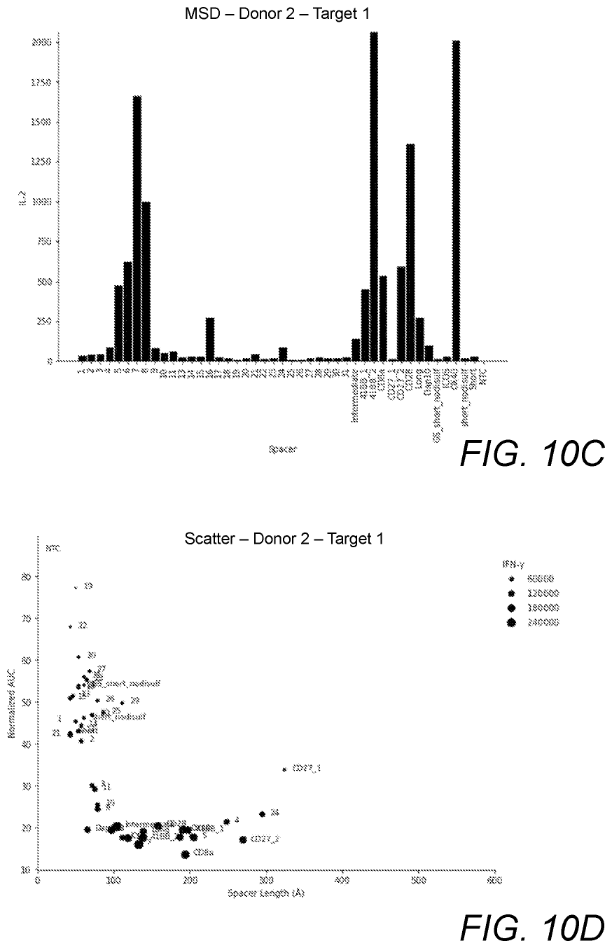

FIGS. 10A, 10B, 10C, and 10D illustrate the effects of indicated spacers on anti HA scFv-induced cytotoxicity in the presence of NucLight Red-labeled A549 target cells expressing the most membrane proximal HA target epitope (A549-HA target 1) in Donor 2. FIG. 10A is a plot showing the Area Under the Curve (AUC) calculated for IncuCyte killing curves of each CAR variant with the indicated spacer. Non-transduced primary T cells are indicated as “NTC.” FIG. 10B and FIG. 10C are bar graphs showing concentrations of IFN-γ and IL-2, respectively, in co-culture media 24 hours post co-culturing. FIG. 10D is a scatter plot summarizing the effects of indicated spacer lengths on target-dependent cytokine secretion and killing AUC for the anti HA CAR-T cells when co-cultured with A549-HA target 1. Dot size represents IFN-7; the larger the dot size, the higher the level of IFN-γ secretion.

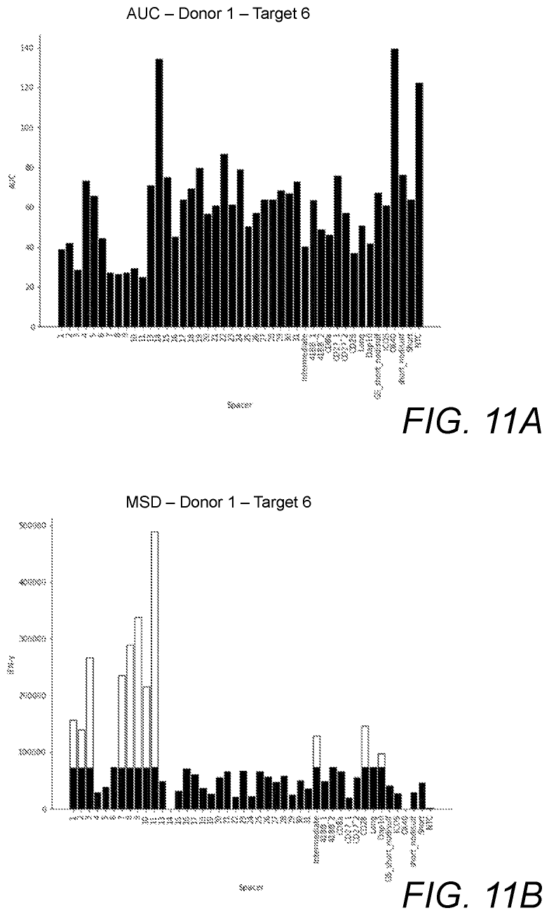

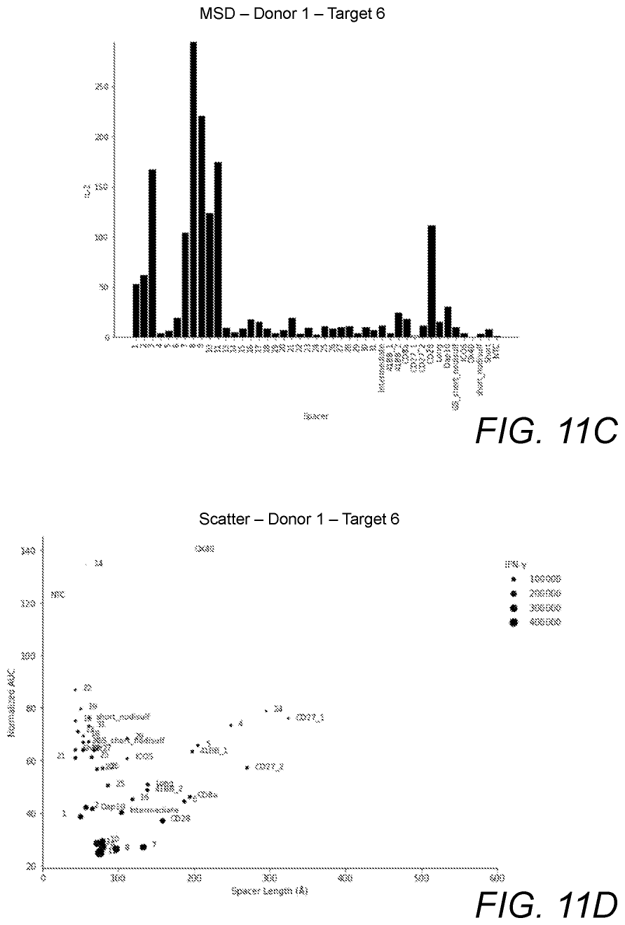

FIGS. 11A, 11B, 11C, and 11D illustrate the effects of indicated spacers on anti HA scFv-induced cytotoxicity in the presence of NucLight Red-labeled A549 target cells expressing a HA target epitope at intermediate membrane distance (A549-HA target 6) in Donor 1. FIG. 11A is a plot showing the Area Under the Curve (AUC) calculated for IncuCyte killing curves of each CAR variant with the indicated spacer. Non-transduced primary T cells are indicated as “NTC.” FIG. 11B and FIG. 11C are bar graphs showing concentrations of IFN-γ and IL-2, respectively, in co-culture media 24 hours post co-culturing. FIG. 11D is a scatter plot summarizing the effects of indicated spacer lengths on target-dependent cytokine secretion and killing AUC for the anti HA CAR-T cells when co-cultured with A549-HA target 6. Dot size represents IFN-γ; the larger the dot size, the higher the level of IFN-γ secretion.

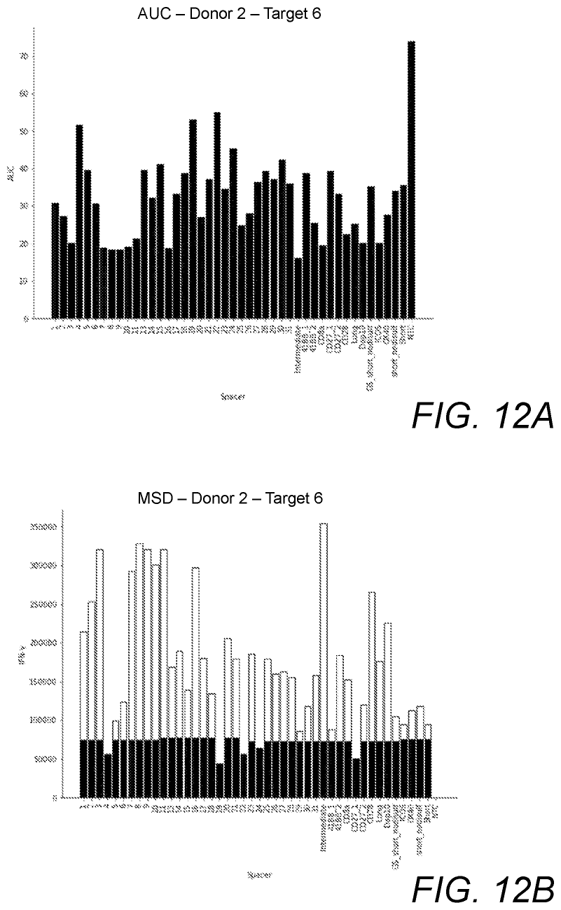

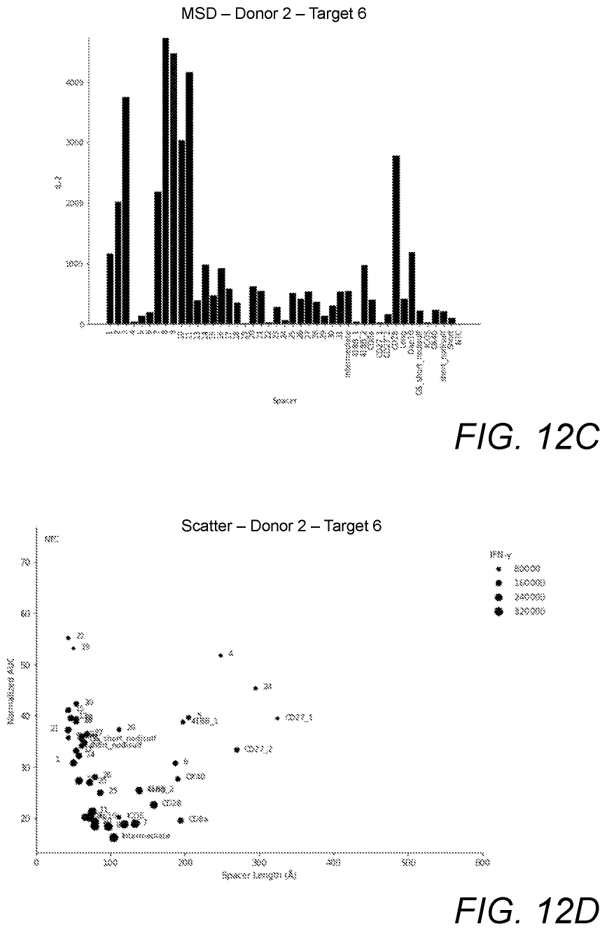

FIGS. 12A, 12B, 12C, and 12D illustrate the effects of indicated spacers on anti HA scFv-induced cytotoxicity in the presence of NucLight Red-labeled A549 target cells expressing a HA target epitope at intermediate membrane distance (A549-HA target 6) in Donor 2. FIG. 12A is a plot showing the Area Under the Curve (AUC) calculated for IncuCyte killing curves of each CAR variant with the indicated spacer. Non-transduced primary T cells are indicated as “NTC.” FIG. 12B and FIG. 12C are bar graphs showing concentrations of IFN-γ and IL-2, respectively, in co-culture media 24 hours post co-culturing. FIG. 12D is a scatter plot illustrating the effects of indicated spacer lengths on target-dependent cytokine secretion and killing AUC for the anti HA CAR-T cells when co-cultured with A549-HA target 6. Dot size represents IFN-γ; the larger the dot size, the higher the level of IFN-γ secretion.

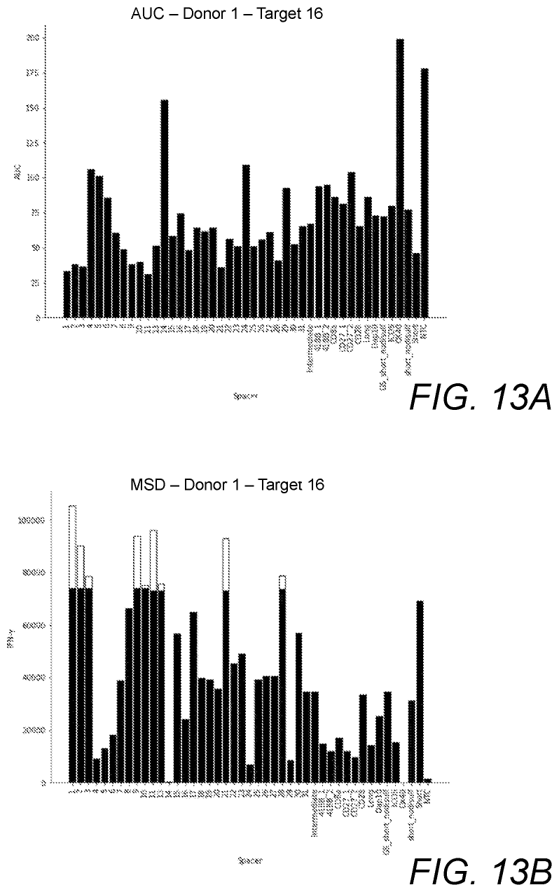

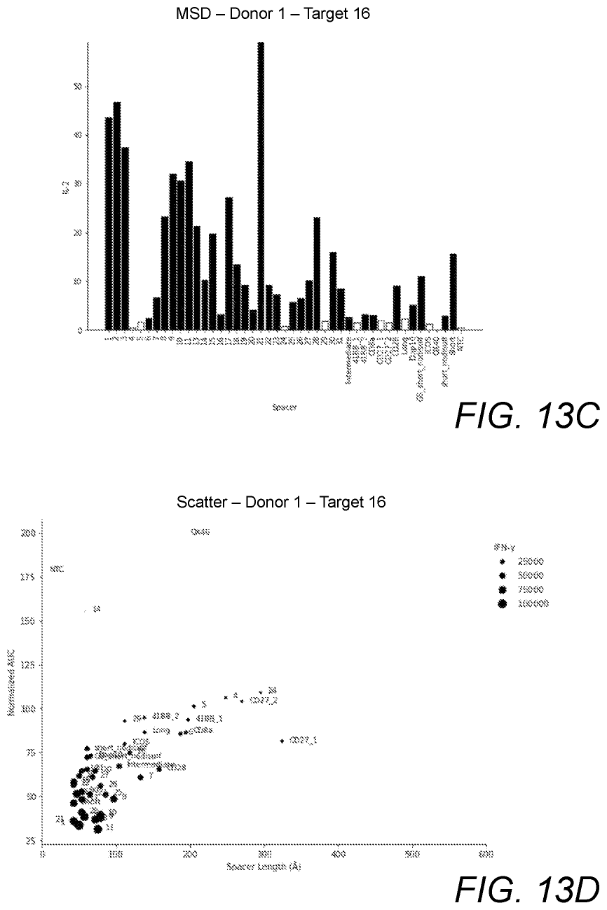

FIGS. 13A, 13B, 13C, and 13D illustrate the effects of indicated spacers on anti HA scFv-induced cytotoxicity in the presence of NucLight Red-labeled A549 target cells expressing the most membrane distal HA target epitope (A549-HA target 16) in Donor 1. FIG. 13A is a plot showing the Area Under the Curve (AUC) calculated for IncuCyte killing curves of each CAR variant with the indicated spacer. Non-transduced primary T cells are indicated as “NTC.” FIG. 13B and FIG. 13C are bar graphs showing concentrations of IFN-γ and IL-2, respectively, in co-culture media 24 hours post co-culturing. FIG. 13D is a scatter plot summarizing the effects of indicated spacer lengths on target-dependent cytokine secretion and killing AUC for the anti HA CAR-T cells when co-cultured with A549-HA target 16. Dot size represents IFN-7; the larger the dot size, the higher the level of IFN-7 secretion.

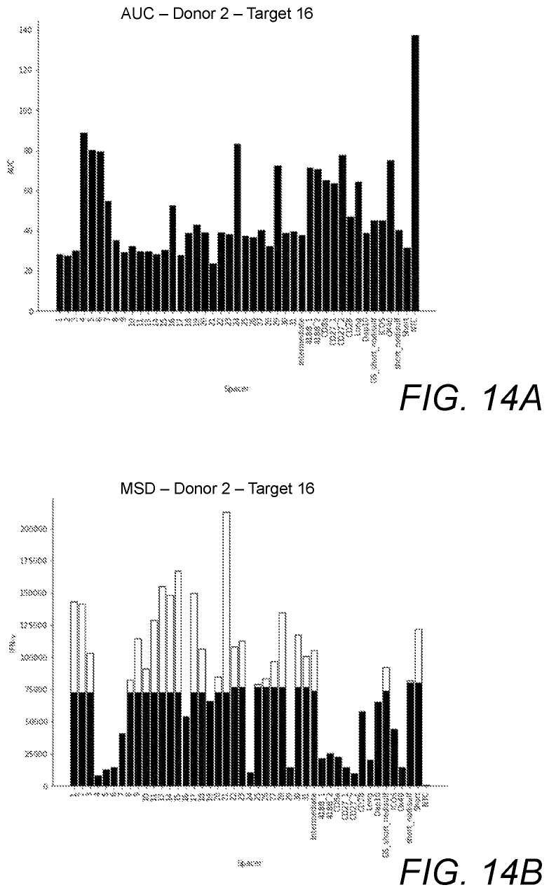

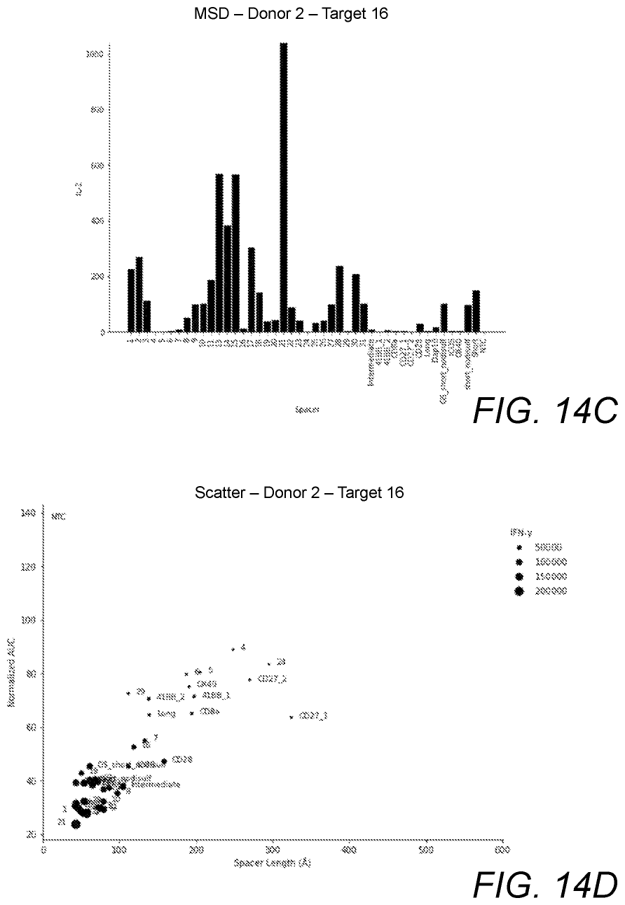

FIGS. 14A, 14B, 14C, and 14D illustrate the effects of indicated spacers on anti HA scFv-induced cytotoxicity in the presence of NucLight Red-labeled A549 target cells expressing the most membrane distal HA target epitope (A549-HA target 16) in Donor 2. FIG. 14A is a plot showing the Area Under the Curve (AUC) calculated for IncuCyte killing curves of each CAR variant with the indicated spacer. Non-transduced primary T cells are indicated as “NTC.” FIG. 14B and FIG. 14C are bar graphs showing concentrations of IFN-γ and IL-2, respectively, in co-culture media 24 hours post co-culturing. FIG. 14D is a scatter plot summarizing the effects of indicated spacer lengths on target-dependent cytokine secretion and killing AUC for the anti HA CAR-T cells when co-cultured with A549-HA target 16. Dot size represents IFN-7; the larger the dot size, the higher the level of IFN-γ secretion.

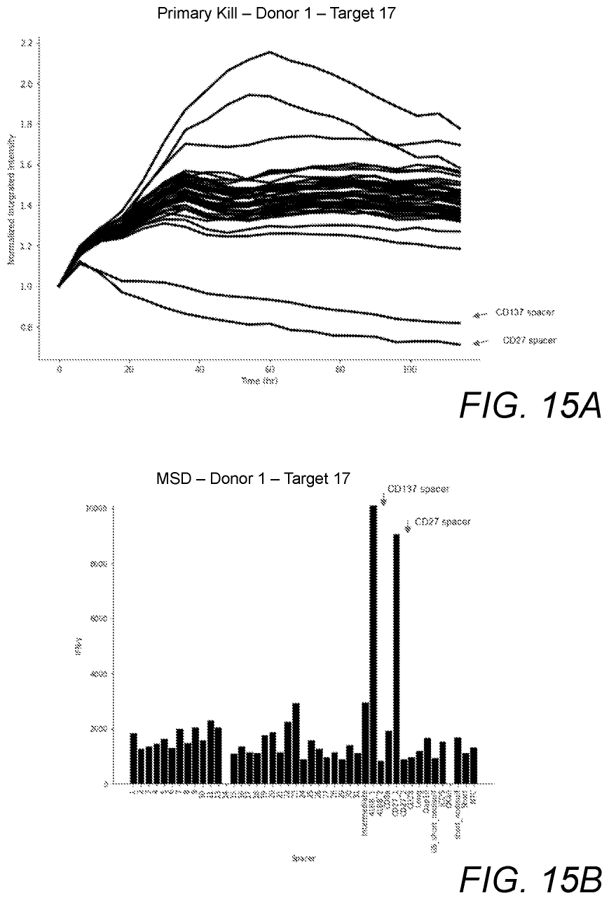

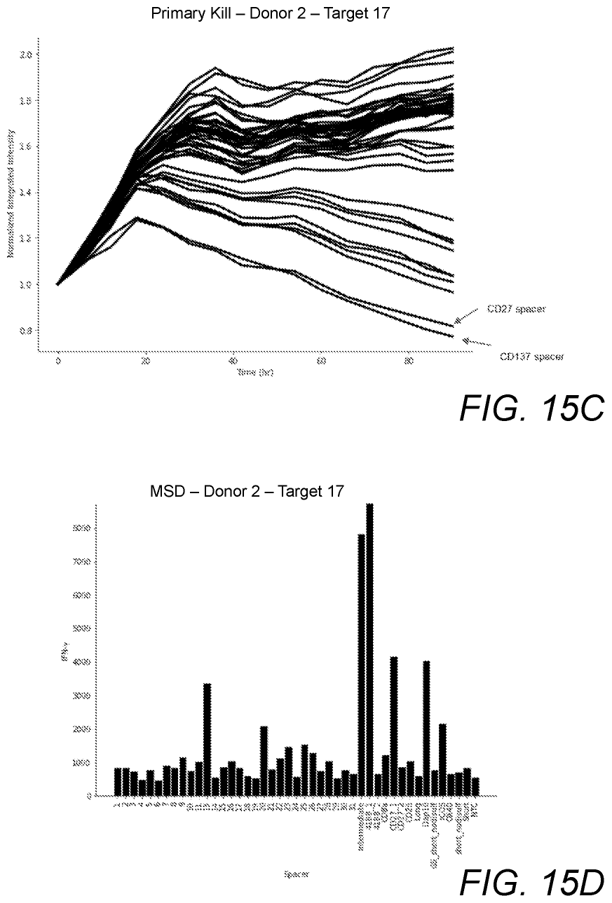

FIGS. 15A, 15B, 15C, and 15D illustrate the effects of indicated spacers on anti HA scFv-induced cytotoxicity in the presence of NucLight Red-labeled A549 target cells without HA target epitope (i.e., antigen negative wildtype A549 cells=Target 17). Referring now to FIG. 15A is a plot showing the IncuCyte kinetic killing curves (NucLight Red signal over time) for each CAR variant derived from Donor 1 with the indicated spacer. Non-transduced primary T cells are indicated as “NTC.” FIG. 15B is a bar graph showing the concentration of IFN-γ in co-culture media 24 hours post co-culturing from Donor 1. The data show that full length 41BB and full length CD27 ECD spacers exhibit elevated IFN-7 production, and cytotoxic activity against antigen negative A549 cells.

Referring now to FIG. 15C is a plot showing the IncuCyte kinetic killing curves (NucLight Red signal over time) for each CAR variant derived from Donor 2 with the indicated spacer. Non-transduced primary T cells are indicated as “NTC.” FIG. 15D is a bar graph showing concentration of IFN-7 in co-culture media 24 hours post co-culturing from Donor 2. The data show that Intermediate spacer, full length 41BB and full length CD27 ECD spacers exhibit elevated IFN-γ production, and cytotoxic activity against antigen negative A549 cells.

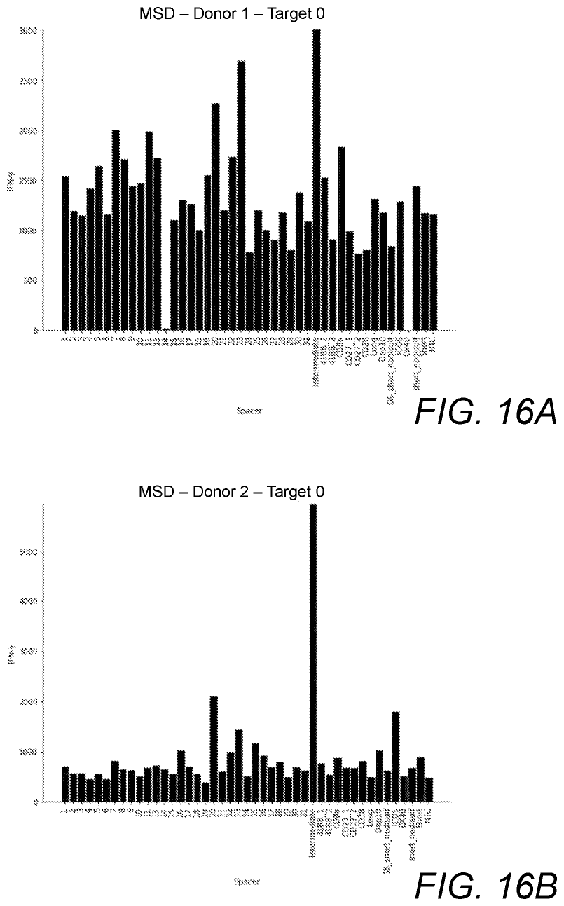

FIG. 16A and FIG. 16B are plots showing IFN-γ secretion profiles on HA CAR-T cells derived from Donor 1 and Donor 2, respectively, expressing indicated spacers in the absence of target cells. The spacers exhibiting elevated cytokine production in the absence of target cells are considered to exhibit tonic signaling.

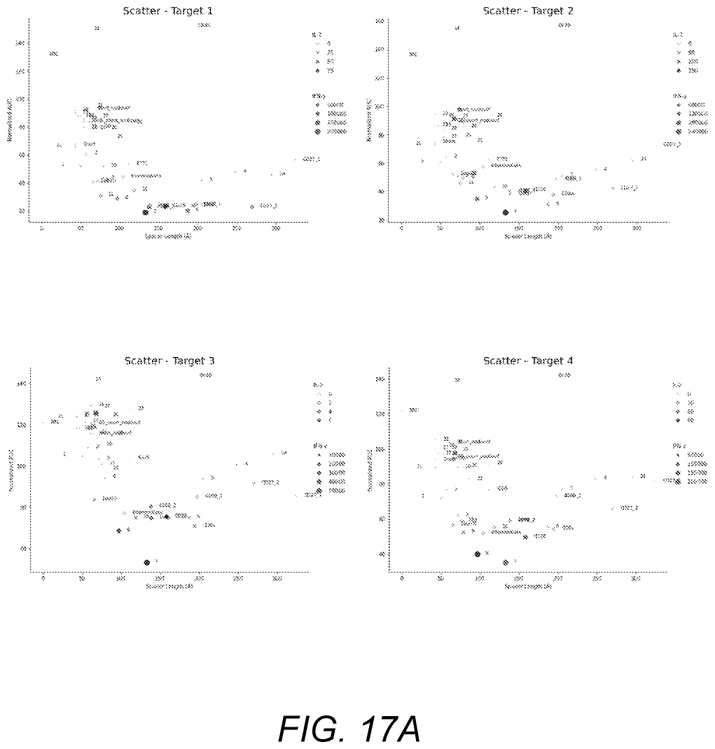

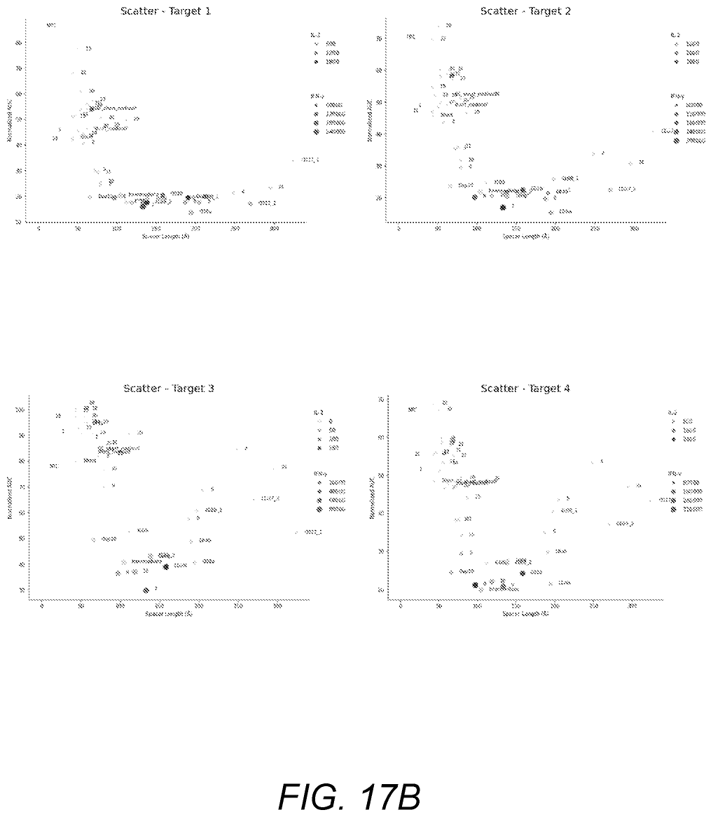

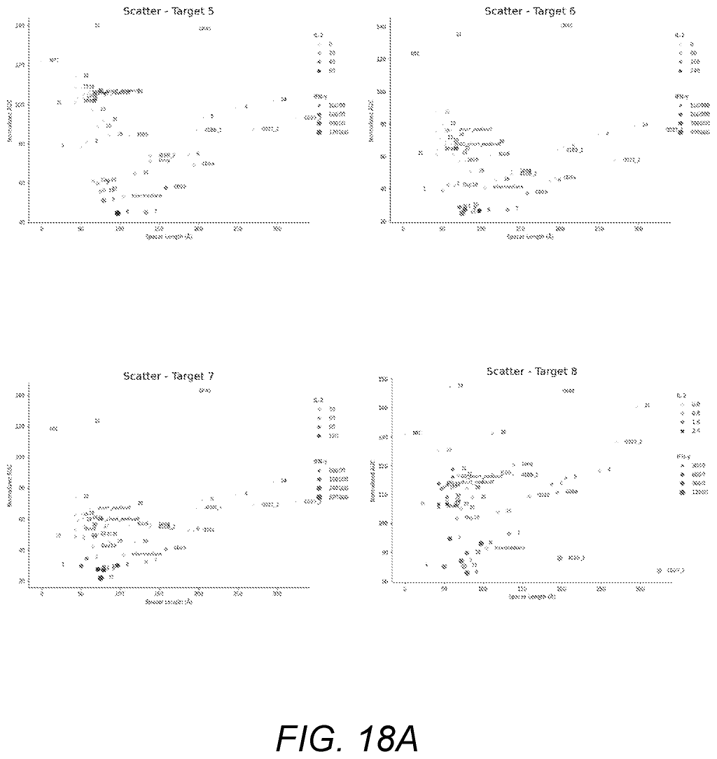

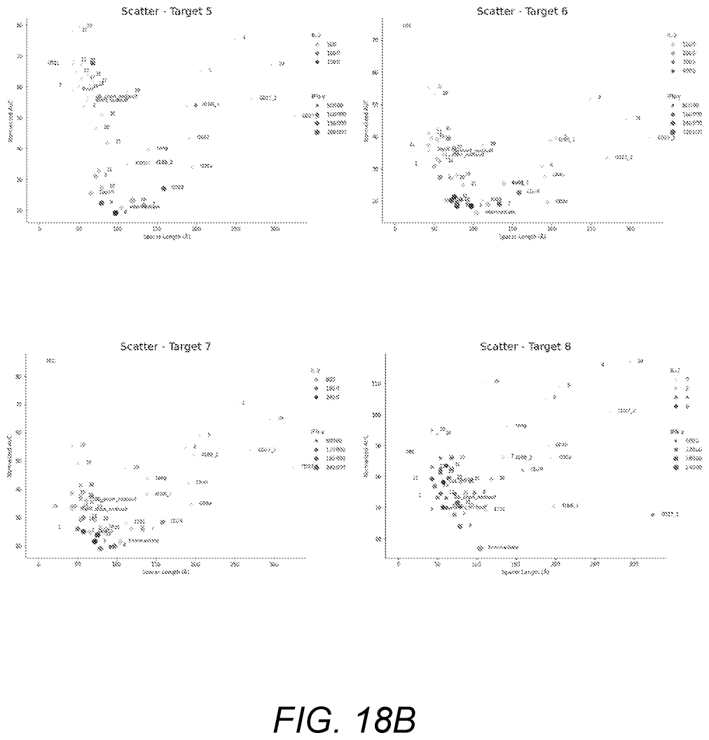

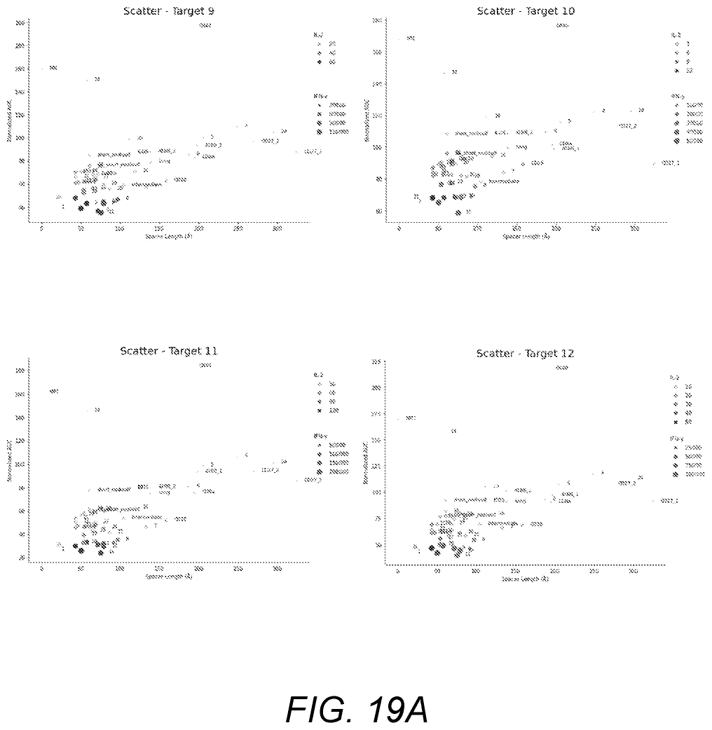

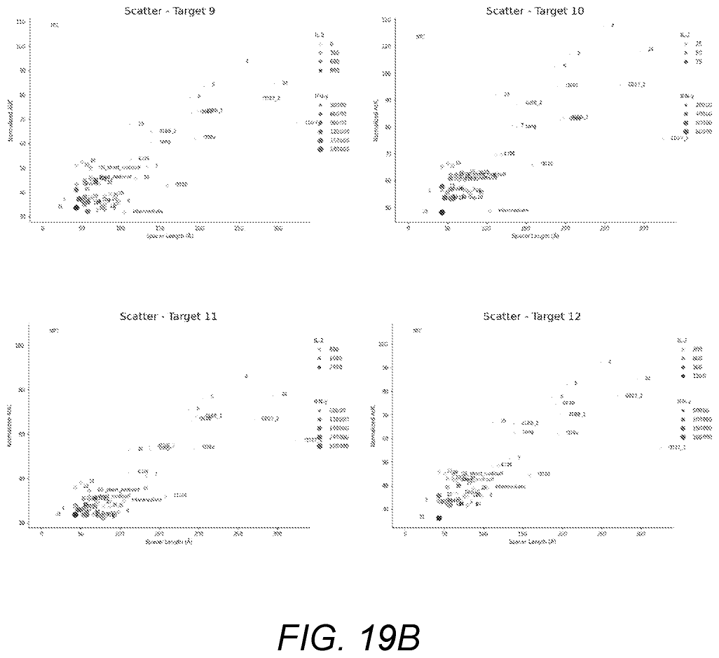

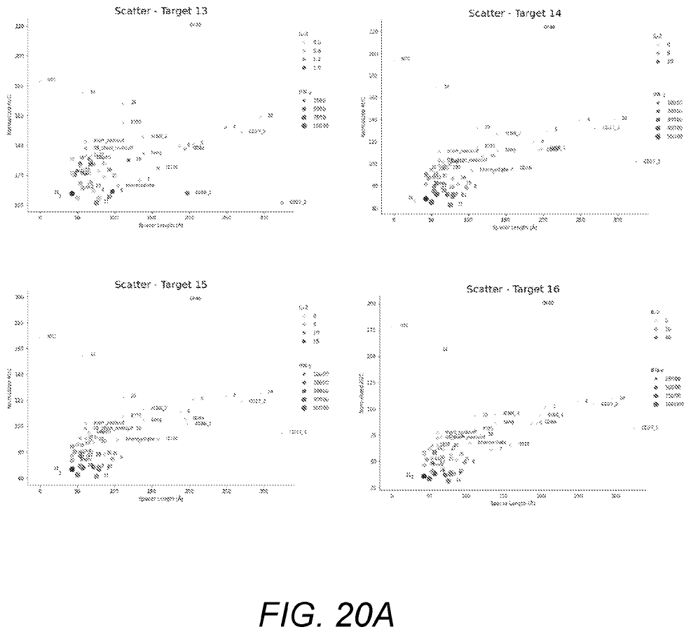

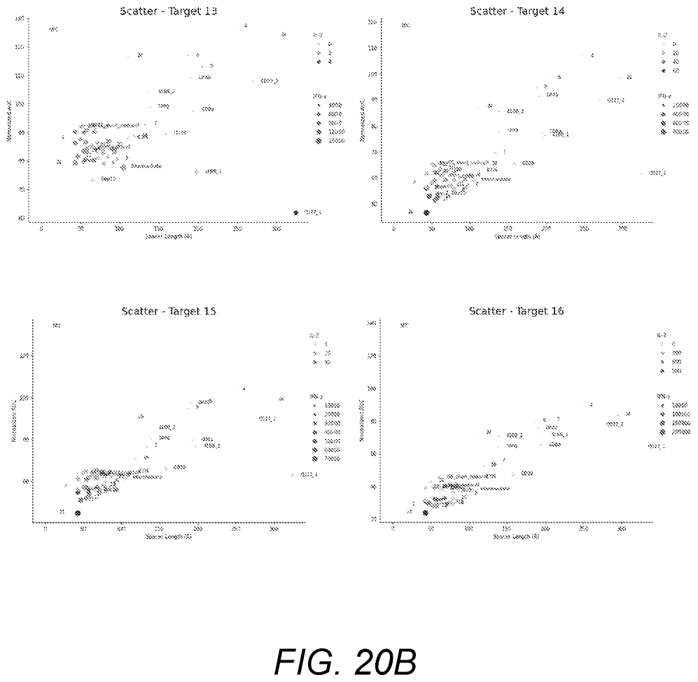

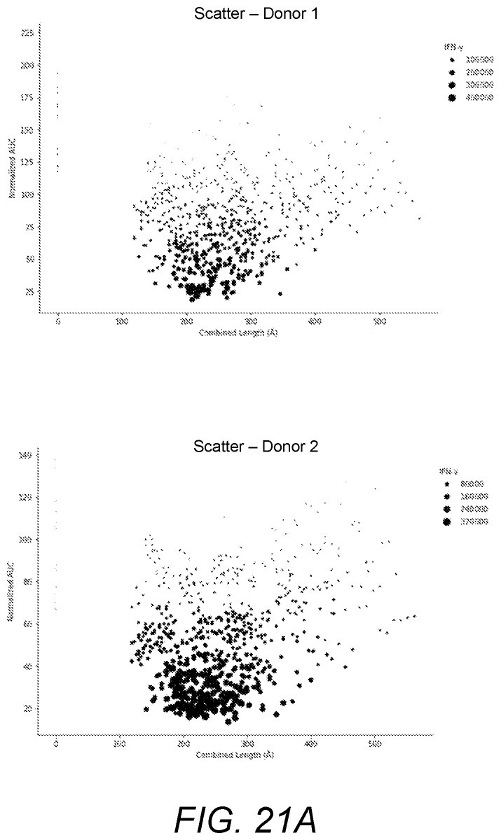

FIG. 17 through FIG. 20 are scatter plots summarizing the effects of systematically varying HA epitope membrane distances on optimal anti-HA CAR spacers lengths on target-dependent cytokine secretion and killing AUC for Donor 1 and Donor 2 CAR T cells co-cultured with a A549-HA target cell (i.e., target cells 1 through 16, see Table 2). Normalized AUC is on the y-axis and spacer length is on the x-axis. The black dots represent IFN-γ; the larger the dot size, the higher the level of IFN-7 secretion. The blue dots represent IL-2; the darker the intensity of the blue color, the higher the level of IL-2 secretion. FIG. 17A, FIG. 18A, FIG. 19A, and FIG. 20A show HA CAR-T derived from Donor 1 co-cultured with A549 target cells expressing HA epitope at increasing distance from target cell membrane. FIG. 17B, FIG. 18B, FIG. 19B, and FIG. 20B show HA CAR-T derived from Donor 2 co-cultured with A549 target cells expressing HA epitope at increasing distance from target cell membrane. The data show that as target epitope distance increases (i.e., from target 1 to 16, see Table 2) from the target cell surface, preferred spacer length decreases. For example, at the longest epitope distance, target 16 (see plot 20B), only the shortest CAR spacer mediates sufficient cytokine secretion and target cell killing. The data also show that changes in IL-2 and IFN-γ secretion follow a similar trend as target epitope distance changes.

As described herein above with reference to FIG. 2, the cell-to-cell distance can be estimated by combined length of the CAR spacer (including GS linker), scFv-epitope binding element binding with the epitope. For the HA-epitope and anti-HA binding element used in this study, the HA-epitope C-term to scFv C-term is 36.5 Å as measured from its crystal structure. The lengths of the CAR spacers are known and shown in Table 1. The lengths of the HA-epitope spacers are also known and are shown in Table 2.

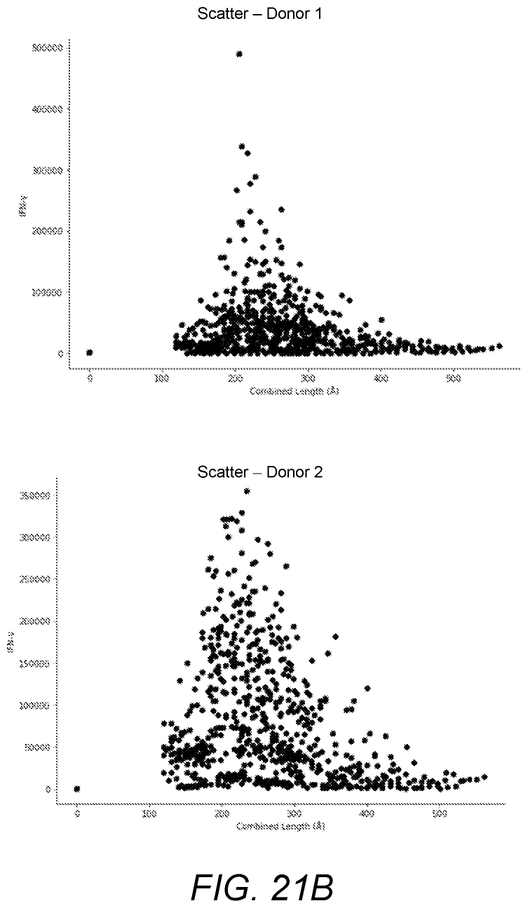

FIG. 21A shows superimposed scatter plots summarizing the effects of cell-to-cell distance on AUC calculated from IncuCyte killing curves and IFN-γ secretion in Donor 1 and Donor 2, respectively, measured 24 hours post co-culturing. Normalized AUC is on the y-axis and combined spacer length is on the x-axis. Dot size represents IFN-γ level; the larger the dot size, the higher the level of IFN-γ secretion. The superimposed scatter plots represent 700+ data points, i.e., data from 700+ pairwise combinations of CAR spacers (n=42, see Table 1) and epitope spacers (n=16, see Table 2). FIG. 21B shows superimposed scatter plots summarizing the effects of cell-to-cell distance on IFN-γ secretion in Donor 1 and Donor 2, respectively. Referring now to FIG. 21A and FIG. 21B, the data show that the highest kill efficacy and cytotoxicity is observed when the combined length equals approximately 200 Å. This result indicates that the optimal interaction between a CART cell and a target cell occurs when the synaptic distance is about 200 Å.

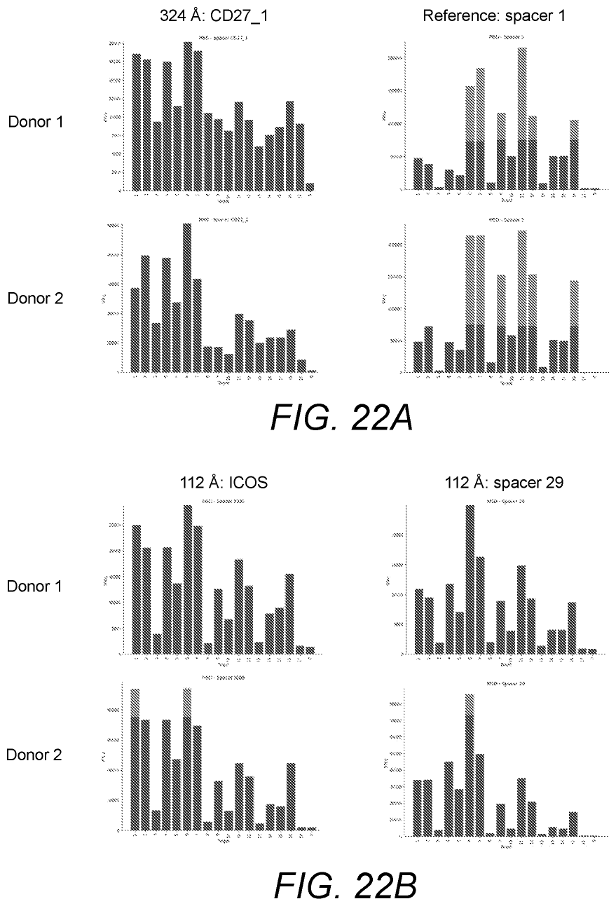

FIG. 22A is a panel of plots showing IFN-γ secretion levels for CAR spacers CD27_1 and spacer 1 expressed in Donor 1 and Donor 2 paired with each target cell line (i.e., targets 1 through 16). Spacer CD27_1 has a length of about 324 Å, which is longer than the optimal cell-to-cell distance for a CAR T cell of about 200 Å. Spacer 1, which has a length of about 50 Å, is used as a reference control. The data show that spacer CD27_1, which is too long for any given target 1-16 results in IFN-γ levels that are sub-maximal, which is likely due to interaction of the spacer with other proteins on the surface of the target cells. In contrast, spacer 1 shows IFN-γ secretion levels that are on-par relative to the distance of the epitope from the surface of the target cell.

FIG. 22B is a panel of plots showing IFN-γ secretion levels for CAR spacers ICOS and spacer 29 expressed in Donor 1 and Donor 2 paired with each target cell line (i.e., targets 1 through 16). Both ICOS and spacer 29 have a length of about 112 Å, which is considered to be a good length for a spacer, i.e., not too short or too long. The data show both the ICOS and spacer 29 result in IFN-γ levels that are about 10-fold less than the reference spacer 1 (see FIG. 22A), which may indicate a sequence composition that is independent of spacer length is affecting the performance of these CAR T cells.

We claim:

1. A Chimeric Antigen Receptor (CAR) comprising
(a) an extracellular element comprising
(i) a binding element that specifically binds to a target and
(ii) a spacer sequence;
(b) a transmembrane element; and
(c) an intracellular element;
wherein, when the CAR is expressed on the surface of a cell, a cell-to-cell distance between a CAR expressing cell and a target expressing cell is between about 17 nm to about 25 nm, inclusive of the endpoints.

2. The CAR of claim 1, wherein, when the CAR is expressed on the surface of a cell, a cell-to-cell distance between a CAR expressing cell and a target expressing cell is between from about 17 nm to about 22 nm.

3. The CAR of claim 1, wherein, when the CAR is expressed on the surface of a cell, a cell-to-cell distance between a CAR expressing cell and a target expressing cell is about 20 nm.

4. The CAR of any one of claims 1-3, wherein the spacer sequence comprises a length of between about 2 nanometers (nm) and about 20 nm, inclusive of the endpoints.

5. The CAR of claim 1, wherein, when the spacer sequence comprises a length of between about 3 nm and about 20 nm, inclusive of the endpoints.

6. The CAR of claim 1, wherein, when the spacer sequence comprises a length of between about 4 nm and about 20 nm, inclusive of the endpoints.

7. The CAR of any one of claims 1-6, wherein the spacer sequence comprises a linker sequence.

8. The CAR of claim 7, wherein the linker comprises a Glycine-Serine (GS) linker.

9. The CAR of claim 7 or 8, wherein the linker sequence comprises GGGSG (SEQ ID NO: 77).

10. The CAR of any one of claims 1-9, wherein the spacer sequence comprises one or more sequences selected from the group consisting of SEQ ID NO: 1-44.

11. The CAR of any one of claims 1-10, wherein the spacer sequence comprises one or more of a deletion, an insertion, a substitution, an inversion, a truncation or a modification.

12. The CAR of any one of claims 1-11, wherein the spacer sequence comprises a sequence having at least 50%, 55%, 60%, 65%, 70%, 75%, 80%, 85%, 80%, 95%, 97%, 99% identity to one or more of SEQ ID NO: 1-44.

13. The CAR of any one of claims 1-12, wherein the spacer sequence comprises at least one non-naturally occurring residue.

14. The CAR of any one of claims 11-13, wherein the substitution comprises a replacement of one first amino acid for a second amino acid, wherein first amino acid and the second amino acid have one or more of polarity, side chain length, or hydrophobicity.

15. The CAR of any one of claims 1-14, wherein the binding element comprises an antigen recognition domain.

16. The CAR of any one of claims 1-14, wherein the binding element comprises a first antigen recognition domain and a second antigen recognition domain.

17. The CAR of any one of claims 1-16, wherein the binding element or the antigen recognition domain comprises an antibody, a protein scaffold, an antibody mimetic, or an antigen binding sequence thereof.

18. The CAR of any one of claims 1-17, wherein the transmembrane element comprises a sequence isolated or derived from a sequence of a CD28 protein.

19. The CAR of any one of claims 1-18, wherein the intracellular element comprises one or more of a costimulatory element and a CD3-zeta signaling element.

20. The CAR of any one of claims 1-19, wherein the binding element or the antigen recognition domain specifically binds to a target, or any portion thereof, isolated or derived from an extracellular antigen.

21. The CAR of claim 20, wherein the target, or any portion thereof, isolated or derived from an extracellular antigen is present or expressed on a surface of a cell.

22. The CAR of any one of claims 1-21, wherein the target expressing cell is in vivo, in vitro, or ex vivo.

23. The CAR of any one of claims 1-21, wherein the target expressing cell comprises one or more modifications.

24. The CAR of claim 23, wherein the target expressing cell is genetically modified.

25. The CAR of any one of claims 1-24, wherein
(a) the binding element that specifically binds to a target, and wherein, when the target sequence is expressed on the surface of a target cell, the target sequence is between about 3 nm and about 4 nm from the surface of the target cell;
the spacer sequence has a length of between about 14 nm and about 19 nm; and
the spacer sequence comprises a sequence of any one of SEQ ID NO: 6, 36, 43, 31, or 7;
(b) the binding element that specifically binds to a target, and wherein, when the target sequence is expressed on the surface of a target cell, the target sequence is between about 5 nm and about 6 nm from the surface of the target cell;
the spacer sequence has a length of between about 12 nm and about 17 nm; and
the spacer sequence comprises a sequence of any one of SEQ ID NO: 36, 43, 31, or 7;
(c) the binding element that specifically binds to a target, and wherein, when the target sequence is expressed on the surface of a target cell, the target sequence is between about 6 nm and about 7 nm from the surface of the target cell;
the spacer sequence has a length of between about 11 nm and about 16 nm; and
the spacer sequence comprises a sequence of any one of SEQ ID NO: 36, 43, 31, 7, 14, 27, or 40;
(d) the binding element that specifically binds to a target, and wherein, when the target sequence is expressed on the surface of a target cell, the target sequence is between about 7 nm and about 8 nm from the surface of the target cell;
the spacer sequence has a length of between about 10 nm and about 15 nm; and
the spacer sequence comprises a sequence of any one of SEQ ID NO: 43, 31, 7, 14, 27, 40, 32 or 42;
(e) the binding element that specifically binds to a target, and wherein, when the target sequence is expressed on the surface of a target cell, the target sequence is between about 8 nm and about 9 nm from the surface of the target cell;
the spacer sequence has a length of between about 9 nm and about 14 nm; and
the spacer sequence comprises a sequence of any one of SEQ ID NO: 43, 31, 7, 14, 27, 40, 32, 42 or 8;
(f) the binding element that specifically binds to a target, and wherein, when the target sequence is expressed on the surface of a target cell, the target sequence is between about 9 nm and about 10 nm from the surface of the target cell;
the spacer sequence has a length of between about 8 nm and about 13 nm; and
the spacer sequence comprises a sequence of anyone of SEQ ID NO: 14, 27, 40, 32, 42, 8 or 23;
(g) the binding element that specifically binds to a target, and wherein, when the target sequence is expressed on the surface of a target cell, the target sequence is between about 10 nm and about 11 nm from the surface of the target cell;
the spacer sequence has a length of between about 7 nm and about 12 nm; and
the spacer sequence comprises a sequence of any one of SEQ ID NO: 14, 27, 40, 32, 42, 8, 23, 9, 10, 24, 11, 3, or 18;
(h) the binding element that specifically binds to a target, and wherein, when the target sequence is expressed on the surface of a target cell, the target sequence is between about 11 nm and about 12 nm from the surface of the target cell;
the spacer sequence has a length of between about 6 nm and about 11 nm; and
the spacer sequence comprises a sequence of any one of SEQ ID NO: 32, 42, 8, 23, 9, 10, 24, 11, 3, 18, 25, 38, 21, 29, 37 or 39;
(i) the binding element that specifically binds to a target, and wherein, when the target sequence is expressed on the surface of a target cell, the target sequence is between about 12 nm and about 13 nm from the surface of the target cell;
the spacer sequence has a length of between about 5 nm and about 10 nm; and
the spacer sequence comprises a sequence of any one of SEQ ID NO: 32, 42, 8, 23, 9, 10, 24, 11, 3, 18, 25, 38, 21, 29, 37, 39, 2, 15, 16, 26, 28, 1, or 17;
(j) the binding element that specifically binds to a target, and wherein, when the target sequence is expressed on the surface of a target cell, the target sequence is between about 13 nm and about 14 nm from the surface of the target cell;
the spacer sequence has a length of between about 4 nm and about 9 nm; and
the spacer sequence comprises a sequence of any one of SEQ ID NO: 23, 9, 10, 24, 11, 3, 18, 25, 38, 21, 29, 37, 39, 2, 15, 16, 26, 28, 1, 17, 12, 13, 19, 20, or 44;
(k) the binding element that specifically binds to a target, and wherein, when the target sequence is expressed on the surface of a target cell, the target sequence is between about 14 nm and about 15 nm from the surface of the target cell;
the spacer sequence has a length of between about 3 nm and about 8 nm; and
the spacer sequence comprises a sequence of any one of SEQ ID NO: 9, 10, 24, 11, 3, 18, 25, 38, 21, 29, 37, 39, 2, 15, 16, 26, 28, 1, 17, 12, 13, 19, 20, or 44;
(1) the binding element that specifically binds to a target, and wherein, when the target sequence is expressed on the surface of a target cell, the target sequence is between about 15 nm and about 16 nm from the surface of the target cell;
the spacer sequence has a length of between about 2 nm and about 7 nm; and
the spacer sequence comprises a sequence of any one of SEQ ID NO: 25, 38, 21, 29, 37, 39, 2, 15, 16, 26, 28, 1, 17, 12, 13, 19, 20, or 44;
(m) the binding element that specifically binds to a target, and wherein, when the target sequence is expressed on the surface of a target cell, the target sequence is between about 16 nm and about 17 nm from the surface of the target cell;
the spacer sequence has a length of between about 1 nm and about 6 nm; and
the spacer sequence comprises a sequence of any one of SEQ ID NO: 2, 15, 16, 26, 28, 1, 17, 12, 13, 19, 20, or 44;
wherein, when the CAR is expressed on the surface of a cell, a cell-to-cell distance between a CAR expressing cell and a target expressing cell is between about 17 nm to 23 nm, inclusive of the endpoints.

26. The CAR of any one of claims 1-24, wherein
(a) the binding element that specifically binds to a target, and wherein, when the target sequence is expressed on the surface of a target cell, the target sequence is between about 3 nm and about 4 nm from the surface of the target cell;
the spacer sequence has a length of between about 15 nm and about 18 nm; and
the spacer sequence comprises a sequence of SEQ ID NO: 36;
(b) the binding element that specifically binds to a target, and wherein, when the target sequence is expressed on the surface of a target cell, the target sequence is between about 5 nm and about 6 nm from the surface of the target cell;
the spacer sequence has a length of between about 13 nm and about 16 nm; and
the spacer sequence comprises a sequence of any one of SEQ ID NO: 36, 43, 31, or 7;
(c) the binding element that specifically binds to a target, and wherein, when the target sequence is expressed on the surface of a target cell, the target sequence is between about 6 nm and about 7 nm from the surface of the target cell;
the spacer sequence has a length of between about 12 nm and about 15 nm; and
the spacer sequence comprises a sequence of any one of SEQ ID NO: 43, 31, or 7;
(d) the binding element that specifically binds to a target, and wherein, when the target sequence is expressed on the surface of a target cell, the target sequence is between about 7 nm and about 8 nm from the surface of the target cell;
the spacer sequence has a length of between about 11 nm and about 14 nm; and
the spacer sequence comprises a sequence of any one of SEQ ID NO: 43, 31, 7, 14, 27, or 40;
(e) the binding element that specifically binds to a target, and wherein, when the target sequence is expressed on the surface of a target cell, the target sequence is between about 8 nm and about 9 nm from the surface of the target cell;
the spacer sequence has a length of between about 10 nm and about 13 nm; and
the spacer sequence comprises a sequence of any one of SEQ ID NO: 14, 27, 40, 32 or 42;
(f) the binding element that specifically binds to a target, and wherein, when the target sequence is expressed on the surface of a target cell, the target sequence is between about 9 nm and about 10 nm from the surface of the target cell;
the spacer sequence has a length of between about 9 nm and about 12 nm; and
the spacer sequence comprises a sequence of any one of SEQ ID NO: 14, 27, 40, 32, 42, 8 or 23;
(g) the binding element that specifically binds to a target, and wherein, when the target sequence is expressed on the surface of a target cell, the target sequence is between about 10 nm and about 11 nm from the surface of the target cell;
the spacer sequence has a length of between about 8 nm and about 11 nm; and
the spacer sequence comprises a sequence of any one of SEQ ID NO: 32, 42, 8 or 23;
(h) the binding element that specifically binds to a target, and wherein, when the target sequence is expressed on the surface of a target cell, the target sequence is between about 11 nm and about 12 nm from the surface of the target cell;
the spacer sequence has a length of between about 7 nm and about 10 nm; and
the spacer sequence comprises a sequence of any one of SEQ ID NO: 8, 23, 9, 10, 24, 11, 3, or 18;
(i) the binding element that specifically binds to a target, and wherein, when the target sequence is expressed on the surface of a target cell, the target sequence is between about 12 nm and about 13 nm from the surface of the target cell;
the spacer sequence has a length of between about 6 nm and about 9 nm; and
the spacer sequence comprises a sequence of any one of SEQ ID NO: 23, 9, 10, 24, 11, 3, 18, 25, 38, 21, 29, 37, or 39;
(j) the binding element that specifically binds to a target, and wherein, when the target sequence is expressed on the surface of a target cell, the target sequence is between about 13 nm and about 14 nm from the surface of the target cell;
the spacer sequence has a length of between about 5 nm and about 8 nm; and
the spacer sequence comprises a sequence of any one of SEQ ID NO: 9, 10, 24, 11, 3, 18, 25, 38, 21, 29, 37, 39, 2, 15, 16, 26, 28, or 17;
(k) the binding element that specifically binds to a target, and wherein, when the target sequence is expressed on the surface of a target cell, the target sequence is between about 14 nm and about 15 nm from the surface of the target cell;
the spacer sequence has a length of between about 4 nm and about 7 nm; and
the spacer sequence comprises a sequence of any one of SEQ ID NO: 25, 38, 21, 29, 37, 39, 2, 15, 16, 26, 28, 17, 12, 13, 19, 20, or 44;
(1) the binding element that specifically binds to a target, and wherein, when the target sequence is expressed on the surface of a target cell, the target sequence is between about 15 nm and about 16 nm from the surface of the target cell;
the spacer sequence has a length of between about 3 nm and about 6 nm; and
the spacer sequence comprises a sequence of any one of SEQ ID NO: 2, 15, 16, 26, 28, 17, 12, 13, 19, 20, or 44;
(m) the binding element that specifically binds to a target, and wherein, when the target sequence is expressed on the surface of a target cell, the target sequence is between about 16 nm and about 17 nm from the surface of the target cell;
the spacer sequence has a length of between about 2 nm and about 5 nm; and
the spacer sequence comprises a sequence of any one of SEQ ID NO: 12, 13, 19, 20, or 44;
wherein, when the CAR is expressed on the surface of a cell, a cell-to-cell distance between a CAR expressing cell and a target expressing cell is between about 18 nm to 22 nm, inclusive of the endpoints.

27. The CAR of any one of claims 1-24, wherein
(a) the binding element that specifically binds to a target, and wherein, when the target sequence is expressed on the surface of a target cell, the target sequence is between about 3 nm and about 4 nm from the surface of the target cell;
the spacer sequence has a length of between about 14 nm and about 21 nm; and
the spacer sequence comprises a sequence of SEQ ID NO: 5, 6, 30, 33, 36 or 41;
(b) the binding element that specifically binds to a target, and wherein, when the target sequence is expressed on the surface of a target cell, the target sequence is between about 5 nm and about 6 nm from the surface of the target cell;
the spacer sequence has a length of between about 12 nm and about 19 nm; and
the spacer sequence comprises a sequence of any one of SEQ ID NO: 6, 7, 31, 36 or 43;
(c) the binding element that specifically binds to a target, and wherein, when the target sequence is expressed on the surface of a target cell, the target sequence is between about 6 nm and about 7 nm from the surface of the target cell;
the spacer sequence has a length of between about 11 nm and about 18 nm; and
the spacer sequence comprises a sequence of any one of SEQ ID NO: 7, 14, 27, 31, 36, 40, or 43;
(d) the binding element that specifically binds to a target, and wherein, when the target sequence is expressed on the surface of a target cell, the target sequence is between about 7 nm and about 8 nm from the surface of the target cell;
the spacer sequence has a length of between about 10 nm and about 17 nm; and
the spacer sequence comprises a sequence of any one of SEQ ID NO: 7, 14, 27, 31, 32, 36, 40, 42 or 43;
(e) the binding element that specifically binds to a target, and wherein, when the target sequence is expressed on the surface of a target cell, the target sequence is between about 8 nm and about 9 nm from the surface of the target cell;
the spacer sequence has a length of between about 9 nm and about 16 nm; and
the spacer sequence comprises a sequence of any one of SEQ ID NO: 7, 8, 14, 27, 31, 32, 36, 40, 42 or 43;
(f) the binding element that specifically binds to a target, and wherein, when the target sequence is expressed on the surface of a target cell, the target sequence is between about 9 nm and about 10 nm from the surface of the target cell;
the spacer sequence has a length of between about 8 nm and about 15 nm; and
the spacer sequence comprises a sequence of any one of SEQ ID NO: 7, 8, 14, 23, 27, 31, 32, 40, 42 or 43;
(g) the binding element that specifically binds to a target, and wherein, when the target sequence is expressed on the surface of a target cell, the target sequence is between about 10 nm and about 11 nm from the surface of the target cell;
the spacer sequence has a length of between about 7 nm and about 14 nm; and
the spacer sequence comprises a sequence of any one of SEQ ID NO: 3, 8, 9, 10, 11, 14, 18, 23, 24, 27, 32, 40, or 42;
(h) the binding element that specifically binds to a target, and wherein, when the target sequence is expressed on the surface of a target cell, the target sequence is between about 11 nm and about 12 nm from the surface of the target cell;
the spacer sequence has a length of between about 6 nm and about 13 nm; and
the spacer sequence comprises a sequence of any one of SEQ ID NO: 3, 8, 9, 10, 11, 14, 18, 21, 23, 24, 25, 27, 29, 32, 37, 38, 39, 40, or 42;
(i) the binding element that specifically binds to a target, and wherein, when the target sequence is expressed on the surface of a target cell, the target sequence is between about 12 nm and about 13 nm from the surface of the target cell;
the spacer sequence has a length of between about 5 nm and about 12 nm; and
the spacer sequence comprises a sequence of any one of SEQ ID NO: 1, 2, 3, 6, 7, 8, 9, 10, 11, 14, 15, 16, 17, 18, 21, 23, 24, 25, 26, 27, 28, 29, 30, 31, 32, 33, 36, 37, 38, 39, 40, 41, 42 or 43;
(j) the binding element that specifically binds to a target, and wherein, when the target sequence is expressed on the surface of a target cell, the target sequence is between about 13 nm and about 14 nm from the surface of the target cell;
the spacer sequence has a length of between about 4 nm and about 11 nm; and
the spacer sequence comprises a sequence of any one of SEQ ID NO: 1, 2, 3, 8, 9, 10, 11, 12, 13, 15, 16, 17, 18, 19, 20, 21, 23, 24, 25, 26, 28, 29, 32, 37, 38, 39, 42 or 44;
(k) the binding element that specifically binds to a target, and wherein, when the target sequence is expressed on the surface of a target cell, the target sequence is between about 14 nm and about 15 nm from the surface of the target cell;
the spacer sequence has a length of between about 3 nm and about 10 nm; and
the spacer sequence comprises a sequence of any one of SEQ ID NO: 1, 2, 3, 8, 9, 10, 11, 12, 13, 15, 16, 17, 18, 19, 20, 21, 23, 24, 25, 26, 28, 29, 37, 38, 39 or 44;
(1) the binding element that specifically binds to a target, and wherein, when the target sequence is expressed on the surface of a target cell, the target sequence is between about 15 nm and about 16 nm from the surface of the target cell;
the spacer sequence has a length of between about 2 nm and about 9 nm; and
the spacer sequence comprises a sequence of any one of SEQ ID NO: 1, 2, 3, 9, 10, 11, 12, 13, 15, 16, 17, 18, 19, 20, 21, 23, 24, 25, 26, 28, 29, 37, 38, 39 or 44;
(m) the binding element that specifically binds to a target, and wherein, when the target sequence is expressed on the surface of a target cell, the target sequence is between about 16 nm and about 17 nm from the surface of the target cell;
the spacer sequence has a length of between about 1 nm and about 8 nm; and
the spacer sequence comprises a sequence of any one of SEQ ID NO: 1, 2, 3, 9, 10, 11, 12, 13, 15, 16, 17, 18, 19, 20, 21, 24, 25, 26, 28, 29, 37, 38, 39 or 44;
(n) the binding element that specifically binds to a target, and wherein, when the target sequence is expressed on the surface of a target cell, the target sequence is between about 18 nm and about 19 nm from the surface of the target cell;
the spacer sequence has a length of between about 1 nm and about 6 nm; and
the spacer sequence comprises a sequence of any one of SEQ ID NO: 1, 2, 12, 13, 15, 16, 17, 19, 20, 26, 28, 29, 37, 38, 39 or 44;
(o) the binding element that specifically binds to a target, and wherein, when the target sequence is expressed on the surface of a target cell, the target sequence is between about 19 nm and about 20 nm from the surface of the target cell;
the spacer sequence has a length of between about 1 nm and about 5 nm; and
the spacer sequence comprises a sequence of any one of SEQ ID NO: 12, 13, 19, 20 or 44;
wherein, when the CAR is expressed on the surface of a cell, a cell-to-cell distance between a CAR expressing cell and a target expressing cell is between about 18 nm to 22 nm, inclusive of the endpoints.

28. The CAR of any one of claims 25-27, wherein the spacer sequence comprises one or more of a deletion, an insertion, a substitution, an inversion, a truncation or a modification.

29. The CAR of any one of claims 25-28, wherein the spacer sequence comprises a sequence having at least 50%, 55%, 60%, 65%, 70%, 75%, 80%, 85%, 80%, 95%, 97%, 99% identity to one or more of SEQ ID NO: 1-44.

30. The CAR of any one of claims 25-29, wherein the spacer sequence comprises at least one non-naturally occurring residue.

31. The CAR of any one of claims 27-30, wherein the substitution comprises a replacement of one first amino acid for a second amino acid, wherein first amino acid and the second amino acid have one or more of polarity, side chain length, or hydrophobicity.

32. A nucleic acid sequence encoding the CAR of any one of claims 1-31, or any element thereof.

33. A vector comprising the nucleic acid of claim 32.

34. A cell comprising the CAR of any one of claims 1-31.

35. A cell comprising the nucleic acid sequence of claim 32.

36. A cell comprising the vector of claim 33.

37. A composition comprising the CAR of any one of claims 1-31, or any element thereof.

38. A composition comprising the nucleic acid of claim 32.

39. A composition comprising the vector of claim 33.

40. A composition comprising a cell of any one of claims 34-36.

41. A pharmaceutical composition comprising a CAR of any one of claims 1-31 and a pharmaceutically acceptable carrier.

42. A pharmaceutical composition comprising a nucleic acid of claim 32 and a pharmaceutically acceptable carrier.

43. A pharmaceutical composition comprising a vector of claim 33 and a pharmaceutically acceptable carrier.

44. A pharmaceutical composition comprising a cell of any one of claims 34-36 and a pharmaceutically acceptable carrier.

45. A pharmaceutical composition comprising a composition of any one of claims 37-40 and a pharmaceutically acceptable carrier.

46. A use of a CAR of any one of claims 1-31, a nucleic acid of claim 32, a vector of claim 33, a cell of any one of claims 34-26, a composition of any one of claims 37-40 or a pharmaceutical composition of any one of claims 41-45 for use in the manufacture of a medicament for treatment or prevention of a disease or disorder.

47. A use of a CAR of any one of claims 1-31, a nucleic acid of claim 32, a vector of claim 33, a cell of any one of claims 34-26, a composition of any one of claims 37-40 or pharmaceutical composition of any one of claims 41-45 for treatment or prevention of a disease or disorder.

48. The use of claim 46 or 47, wherein the disease or disorder comprises a cancer.

49. A method of treating a disease or disorder, comprising administering to a subject a therapeutically-effective amount of a CAR of any one of claims 1-31, a nucleic acid of claim 32, a vector of claim 33, a cell of any one of claims 34-26, a composition of any one of claims 37-40 or a pharmaceutical composition of any one of claims 41-45, wherein a severity of a sign or symptom of the disease or disorder is decreased, thereby treating the disease or disorder.

50. The method of claim 49, wherein the disease or disorder comprises a cancer.

51. A method of preventing a disease or disorder, comprising administering to a subject a therapeutically-effective amount of a CAR of any one of claims 1-31, a nucleic acid of claim 32, a vector of claim 33, a cell of any one of claims 34-26, a composition of any one of claims 37-40 or a pharmaceutical composition of any one of claims 41-45, wherein an onset or a relapse of a sign or symptom of the disease or disorder is delayed or inhibited, thereby preventing the disease or disorder.

52. The method of claim 51, wherein the disease or disorder comprises a cancer.
